(19) United States
(12) Patent Application Publication
(10) Pub. No.: US 2022/0136020 A1
(43) Pub. Date: May 5, 2022

(54) Methods for purifying bacterial polysaccharides

(71) Applicant: Pfizer Inc., New York, NY (US)

(72) Inventors: Ling Chu, Suffern, NY (US); Scott Andrew Cook, Ballwin, MO (US); Nishith Merchant, Parlin, NJ (US); Justin Keith Moran, West Nyack, NY (US)

(73) Assignee: Pfizer Inc., New York, NY (US)

(21) Appl. No.: 17/431,462

(22) PCT Filed: Feb. 20, 2020
(86) PCT No.: PCT/IB2020/051430

§ 371 Date: Aug. 17, 2021

Related U.S. Application Data
(60) Provisional application No. 62/808,907, filed on Feb. 22, 2019.

(51) Int. Cl. C12P 19/04; B01D 21/01; B01D 61/14; B01D 39/20

(52) U.S. Cl. CPC C12P 19/04; B01D 21/01; B01D 39/2068; B01D 61/147; B01D 61/145

(57) ABSTRACT

The present invention relates to methods for purifying bacterial polysaccharides, in particular for removing impurities from cellular lysates of bacteria producing polysaccharides.

### FIELD OF THE INVENTION

The present invention relates to methods for purifying bacterial polysaccharides, in particular for removing impurities from cellular lysates of bacteria producing polysaccharides.

### BACKGROUND OF THE INVENTION

Bacterial polysaccharides, in particular capsular polysaccharides, are important immunogens found on the surface of bacteria involved in various bacterial diseases. This has led to them being an important component in the design of vaccines. They have proved useful in eliciting immune responses especially when linked to carrier proteins.

Bacterial polysaccharides are typically produced by fermentation of the bacteria (e.g.

Streptococci (e.g., S. pneumoniae, S. pyogenes, S. agalactiae or Group C & G Streptococci), Staphylococci (e.g., Staphylococcus aureus), Haemophilus, (e.g., Haemophilus influenzae), Neisseria (e.g., Neisseria meningitidis) and Escherichia, (e.g., Escherichia coli)).

Typically, bacterial polysaccharides are produced using batch culture in complex medium, fed batch culture or continuous culture.

There is a need for robust and efficacious purification processes that can be used in the large-scale production of bacterial polysaccharides post-fermentation.

Most of the processes include a step of precipitation of the capsular polysaccharide (e.g. alcoholic precipitation or cationic detergent treatment). The subsequent separation of the precipitate from the supernatant (e.g. by centrifugation) and re-solubilization is laborious and may result in loss of polysaccharide, thereby reducing yield.

Furthermore, most of the purification process requires several steps involving many expensive, labor intensive and technologically demanding operations, such as chromatography and multiple membrane separations. The removal of impurities in these processes is spread over many labor intensive and costly steps. Protein level is the most problematic specification to meet due to the physical and chemical properties of the soluble proteins.

Thus, there is a need for a simplified purification process to reduce the soluble protein levels in bacterial lysates and eliminate inefficiencies of the current purification process to produce substantially purified bacterial saccharides suitable for incorporation into vaccines.

### FIGURES

FIG. 1 Process Flow Diagram for Purification of polysacharide

FIG. 2 Effect of pH at 2% w/v alum on protein removal and clarity of S. pneumoniae serotype 8 fermentation broth at various time points. After 1 hour (left bar), 4 hours (middle bar), 24 hours (right bar)

FIG. 3 Effect of % alum at pH 3.5 on protein removal and clarity of S. pneumoniae Serotype 8 fermentation broth at various time points. 1.0% Alum (left bar), 2.0% Alum (middle bar), 3.0% Alum (right bar)

FIG. 4 Acid titration of S. pneumoniae serotype 33F fermentation Broth

FIG. 5 Alum Flocculation of S. pneumoniae serotype 33F at pH 3.5

FIG. 6 Effect of Heating on S. pneumoniae serotype 22F Flocculated broth Particle Size. A For the experiment the flocculation temperature was held at room temperature (RT) (smaller particle size distribution curve (peack at 9.8 μm)) and or at 45° C. (bigger particle size distribution curve (peack at 65 μm))

1. Purification Process of Bacterial Polysaccharides

1.1 Starting Material

The methods of the invention can be used to purify bacterial polysaccharides from a solution comprising said polysaccharides together with contaminants.

1.1.1 Bacterial Cells

The sources of bacterial polysaccharide to be purified according to this invention are bacterial cells, in particular pathogenic bacteria.

Non-limiting examples of gram-positive bacteria for use according to this invention are Streptococci (e.g., S. pneumoniae, S. pyogenes, S. agalactiae or Group C & G Streptococci), Staphylococci (e.g., Staphylococcus aureus), Enterococci, Bacillus, Corynebacterium, Listeria, Erysipelothrix, and Clostridium. Non-limiting examples of gram-negative bacteria for use with this invention include Haemophilus, (e.g., Haemophilus influenzae), Neisseria (e.g., Neisseria meningitidis) and Escherichia, (e.g., Escherichia coli).

In an embodiment, the source of bacterial polysaccharides for use according to this invention is selected from the group consisting of Aeromonas hydrophila and other species (spp.); Bacillus anthracis; Bacillus cereus; Botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia psittaci; Chlamydia trachomatis, Clostridium botulinum; Clostridium dificile; Clostridium perfringens; Coccidioides immitis; Coccidioides posadasii; Cowdria ruminantium (Heartwater); Coxiella burnetii; Enterococcus faecalis; Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), and Escherichia coli—enteroinvasive (EIEC); Ehrlichia spp. such as Ehrlichia chajfeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Peronosclerosporaphilippinensis; Phakopsora pachyrhizi; Plesiomonas shigelloides; Ralstonia solanacearum race 3, biovar 2; Rickettsia prowazekii; Rickettsia rickettsii; Salmonella spp.; Schlerophthora rayssiae var zeae; Shigella spp.; Staphylococcus aureus; Streptococcus; Synchytrium endobioticum; Vibrio cholerae non-01; Vibrio cholerae 01; Vibrio par ahaemo ly ticus and other Vibrios; Vibrio vulnificus; Xanthomonas oryzae; Xylella fastidiosa (citrus variegated chlorosis strain); Yersinia enterocolitica and Yersinia pseudotuberculosis; and Yersinia pestis.

A polysaccharide desired for purification may be associated with a cellular component, such as a cell wall. Association with the cell wall means that the polysaccharide is a component of the cell wall itself, and/or is attached to the cell wall, either directly or indirectly via intermediary molecules, or is a transient coating of the cell wall (for example, certain bacterial strains exude capsular polysaccharides, also known in the art as ‘exopolysaccharides’).

In some embodiments, the polysaccharide extracted from the bacteria is a capsular polysaccharide, a sub-capsular polysaccharide, or a lipopolysaccharide.

In preferred embodiments, the polysaccharide is a capsular polysaccharide.

In an embodiment, the source of bacterial capsular polysaccharide is Staphylococcus aureus. In an embodiment the source of bacterial capsular polysaccharide is Staphylococcus aureus type 5 or Staphylococcus aureus type 8.

In a further embodiment, the source of bacterial capsular polysaccharide is Enterococcus faecalis. In yet a further embodiment, the source of bacterial capsular polysaccharide is Haemophilus influenzae type b.

In a further embodiment, the source of bacterial capsular polysaccharides is Neisseria meningitidis. In an embodiment the source of bacterial capsular polysaccharides is N. meningitidis serogroup A (MenA), N. meningitidis serogroup W135 (MenW135), N. meningitidis serogroup Y (MenY), N. meningitidis serogroup X (MenX) or N. meningitidis serogroup C (MenC). In an embodiment the source of bacterial capsular polysaccharides is N. meningitidis serogroup A (MenA). In an embodiment the source of bacterial capsular polysaccharides is N. meningitidis serogroup W135 (MenW135). In an embodiment the source of bacterial capsular polysaccharides is N. meningitidis serogroup Y (MenY). In an embodiment the source of bacterial capsular polysaccharides is N. meningitidis serogroup C (MenC). In an embodiment the source of bacterial capsular polysaccharides is N. meningitidis serogroup X (MenX).

In a further embodiment, the source of bacterial capsular polysaccharide is Escherichia coll. In a further embodiment, the source of bacterial capsular polysaccharide is Enterococcus faecalis.

In a further embodiment, the source of bacterial capsular polysaccharide is Streptococcus agalactiae (Group B streptococcus (GBS)). In some embodiments, the source of bacterial capsular polysaccharide is selected from the group consisting of GBS types Ia, Ib, II, III, IV, V, VI, VII and VIII. In some embodiments, the source of bacterial capsular polysaccharide is selected from the group consisting of GBS types Ia, Ib, II, III and V.

In a further embodiment, the source of bacterial capsular polysaccharide is Escherichia coll. In an embodiment, the source of bacterial capsular polysaccharide is an Escherichia coli part of the Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), or Escherichia coli—enteroinvasive (EIEC). In an embodiment, the source of bacterial capsular polysaccharide is an Uropathogenic Escherichia coli (UPEC).

In an embodiment, the source of bacterial capsular polysaccharide is an Escherichia coli serotype selected from the group consisting of serotypes O157:H7, O26:H11, O111:H- and O103:H2. In an embodiment, the source of bacterial capsular polysaccharide is an Escherichia coli serotype selected from the group consisting of serotypes O6:K2:H1 and O18:K1:H7. In an embodiment, the source of bacterial capsular polysaccharide is an Escherichia coli serotype selected from the group consisting of serotypes O45:K1, O17:K52:H18, O19:H34 and O7:K1. In an embodiment, the source of bacterial capsular polysaccharide is an Escherichia coli serotype O104:H4. In an embodiment, the source of bacterial capsular polysaccharide is an Escherichia coli serotype O1:K12:H7. In an embodiment, the source of bacterial capsular polysaccharide is an Escherichia coli serotype O127:H6. In an embodiment, the source of bacterial capsular polysaccharide is an Escherichia coli serotype O139:H28. In an embodiment, the source of bacterial capsular polysaccharide is an Escherichia coli serotype O128:H2.

In a preferred embodiment, the source of bacterial capsular polysaccharides is Steptococcus pneumoniae. Preferably the source of bacterial capsular polysaccharides is a Streptococcus pneumoniae serotype selected from the group consisting of serotypes 1, 2, 3, 4, 5, 6A, 6B, 6C, 7F, 8, 9V, 9N, 10A, 11A, 12F, 14, 15A, 15B, 15C, 16F, 17F, 18C, 19A, 19F, 20, 22F, 23A, 23B, 23F, 24B, 24F, 29, 31, 33F, 34, 35B, 35F, 38, 72 and 73. In an embodiment the source of bacterial capsular polysaccharides is a Streptococcus pneumoniae serotype selected from the group consisting of serotypes 1, 2, 3, 4, 5, 6A, 6B, 7F, 8, 9V, 9N, 10A, 11A, 12F, 14, 15A, 15B, 15C, 16F, 17F, 18C, 19A, 19F, 20, 22F, 23A, 23B, 23F, 24F, 29, 31, 33F, 35B, 35F, 38, 72 and 73. In an embodiment the source of bacterial capsular polysaccharides is a Streptococcus pneumoniae serotype selected from the group consisting of serotypes 8, 10A, 11A, 12F, 15B, 22F and 33F. In an embodiment, the source of bacterial capsular polysaccharides is a Streptococcus pneumoniae serotype 1. In an embodiment, the source of bacterial capsular polysaccharides is a Streptococcus pneumoniae serotype 2. In an embodiment, the source of bacterial capsular polysaccharides is a Streptococcus pneumoniae serotype 3. In an embodiment, the source of bacterial capsular polysaccharides is a Streptococcus pneumoniae serotype 4. In an embodiment, the source of bacterial capsular polysaccharides is a Streptococcus pneumoniae serotype 5. In an embodiment, the source of bacterial capsular polysaccharides is a Streptococcus pneumoniae serotype 6A. In an embodiment, the source of bacterial capsular polysaccharides is a Streptococcus pneumoniae serotype 6B. In an embodiment, the source of bacterial capsular polysaccharides is a Streptococcus pneumoniae serotype 6C. In an embodiment, the source of bacterial capsular polysaccharides is a Streptococcus pneumoniae serotype 7F. In an embodiment, the source of bacterial capsular polysaccharides is a Streptococcus pneumoniae serotype 8. In an embodiment, the source of bacterial capsular polysaccharides is a Streptococcus pneumoniae serotype 9V. In an embodiment, the source of bacterial capsular polysaccharides is a Streptococcus pneumoniae serotype 9N. In an embodiment, the source of bacterial capsular polysaccharides is a Streptococcus pneumoniae serotype 10A. In an embodiment, the source of bacterial capsular polysaccharides is a Streptococcus pneumoniae serotype 11A. In an embodiment, the source of bacterial capsular polysaccharides is a Streptococcus pneumoniae serotype 12F. In an embodiment, the source of bacterial capsular polysaccharides is a Streptococcus pneumoniae serotype 14. In an embodiment, the source of bacterial capsular polysaccharides is a Streptococcus pneumoniae serotype 15A. In an embodiment, the source of bacterial capsular polysaccharides is a Streptococcus pneumoniae serotype 15B. In an embodiment, the source of bacterial capsular polysaccharides is a Streptococcus pneumoniae serotype 15C. In an embodiment, the source of bacterial capsular polysaccharides is a Streptococcus pneumoniae serotype 16F. In an embodiment, the source of bacterial capsular polysaccharides is a Streptococcus pneumoniae serotype 17F. In an embodiment, the source of bacterial capsular polysaccharides is a Streptococcus pneumoniae serotype 18C. In an embodiment, the source of bacterial capsular polysaccharides is a Streptococcus pneumoniae serotype 19A. In an embodiment, the source of bacterial capsular polysaccharides is a Streptococcus pneumoniae serotype 19F. In an embodiment, the source of bacterial capsular polysaccharides is a Streptococcus pneumoniae serotype 20. In an embodiment, the source of bacterial capsular polysaccharides is a Streptococcus pneumoniae serotype 20A. In an embodiment, the source of bacterial capsular polysaccharides is a Streptococcus pneumoniae serotype 20B. In an embodiment, the source of bacterial capsular polysaccharides is a Streptococcus pneumoniae serotype 22F. In an embodiment, the source of bacterial capsular polysaccharides is a Streptococcus pneumoniae serotype 23A. In an embodiment, the source of bacterial capsular polysaccharides is a Streptococcus pneumoniae serotype 23B. In an embodiment, the source of bacterial capsular polysaccharides is a Streptococcus pneumoniae serotype 23F. In an embodiment, the source of bacterial capsular polysaccharides is a Streptococcus pneumoniae serotype 24B. In an embodiment, the source of bacterial capsular polysaccharides is a Streptococcus pneumoniae serotype 24F. In an embodiment, the source of bacterial capsular polysaccharides is a Streptococcus pneumoniae serotype 29. In an embodiment, the source of bacterial capsular polysaccharides is a Streptococcus pneumoniae serotype 31. In an embodiment, the source of bacterial capsular polysaccharides is a Streptococcus pneumoniae serotype 33F. In an embodiment, the source of bacterial capsular polysaccharides is a Streptococcus pneumoniae serotype 34. In an embodiment, the source of bacterial capsular polysaccharides is a Streptococcus pneumoniae serotype 35B. In an embodiment, the source of bacterial capsular polysaccharides is a Streptococcus pneumoniae serotype 35F. In an embodiment, the source of bacterial capsular polysaccharides is a Streptococcus pneumoniae serotype 38. In an embodiment, the source of bacterial capsular polysaccharides is a Streptococcus pneumoniae serotype 72. In an embodiment, the source of bacterial capsular polysaccharides is a Streptococcus pneumoniae serotype 73.

Bacterial strains used to purify the respective polysaccharides that are used in the present invention may be obtained from established culture collections or clinical specimens.

1.1.2 Bacterial Cells Growth

Typically the polysaccharides are produced by growing the bacteria in a medium (e.g. a solid or preferably a liquid medium). The polysaccharides are then prepared by treating the bacterial cells.

Therefore in an embodiment, the starting material for methods of the present invention is a bacterial culture and preferably a liquid bacterial culture (e.g. a fermentation broth). The bacterial culture is typically obtained by batch culture, fed batch culture or continuous culture (see e.g. WO 2007/052168 or WO 2009/081276). During continuous culture, fresh medium is added to a culture at a fixed rate and cells and medium are removed at a rate that maintains a constant culture volume.

The population of the organism is often scaled up from a seed vial to seed bottles and passaged through one or more seed fermentors of increasing volume until production scale fermentation volumes are reached.

1.1.3 Pre-Treatment of the Bacterial Cells in Order to Obtain the Starting Material

Generally, a small amount of polysaccharide is released into the culture medium during bacterial growth, and so the starting material may thus be the supernatant from a centrifuged bacterial culture.

Typically, however, the starting material will be prepared by treating the bacteria themselves, such that the polysaccharide is released.

Optionally, after cell growth, the bacterial cells are deactivated. This is particularly the case when pathogenic bacteria are used. A suitable method for deactivation is for example treatment with phenol:ethanol, e.g. as described in Fattom et al. (1990) Infect Immun. 58(7):2367-74. In the below embodiments, the bacterial cells may be previously deactivated or not deactivated.

Polysaccharides can be released from bacteria by various methods, including chemical, physical or enzymatic treatment (see e.g.; WO2010151544, WO 2011/051917 or WO2007084856).

In an embodiment, the bacterial cells (deactivated or not deactivated) are treated in suspension in their original culture medium. The process may therefore start with the cells in suspension in their original culture medium.

In another embodiment the bacterial cells are centrifuged prior to release of capsular polysaccharide. The process may therefore start with the cells in the form of a wet cell paste. Alternatively, the cells are treated in a dried form. Typically, however, after centrifugation the bacterial cells are resuspended in an aqueous medium that is suitable for the next step in the process, e.g. in a buffer or in distilled water. The cells may be washed with this medium prior to re-suspension.

In an embodiment, the bacterial cells (e.g. in suspension in their original culture medium, in the form of a wet cell paste, in a dried form or resuspended in an aqueous medium after centrifugation) are treated with a lytic agent.

A “lytic agent” is any agent that aids in cell wall breakdown.

In an embodiment, the lytic agent is a detergent. As used herein, the term “detergent” refers to any anionic or cationic detergent capable of inducing lysis of bacterial cells. Representative examples of such detergents for use within the methods of the present invention include deoxycholate sodium (DOC), N-lauryl sarcosine (NLS), chenodeoxycholic acid sodium, and saponins (see WO 2008/118752 pages 13 lines 14 to page 14 line 10). In one embodiment of the present invention, the lytic agent used for lysing bacterial cells is DOC.

In an embodiment, the lytic agent is a non-animal derived lytic agent. In one embodiment, the non-animal derived lytic agent is selected from the group consisting of decanesulfonic acid, tert-octylphenoxy 5 poly(oxyethylene)ethanols (e.g. Igepal® CA-630, CAS #: 9002-93-1, available from Sigma Aldrich, St. Louis, Mo.), octylphenol ethylene oxide condensates (e.g. Triton® X-100, available from Sigma Aldrich, St. Louis, Mo.), N-lauryl sarcosine sodium (NLS), lauryl iminodipropionate, sodium dodecyl sulfate, chenodeoxycholate, hyodeoxycholate, glycodeoxycholate, taurodeoxycholate, taurochenodeoxycholate, and cholate. In an embodiment, the non-animal derived lytic agent is NLS.

In an embodiment, the bacterial cells (e.g. in suspension in their original culture medium, in the form of a wet cell paste, in a dried form or resuspended in an aqueous medium after centrifugation) are enzymatically treated such that the polysaccharide is released. In an embodiment, the bacterial cells are treated by an enzyme selected from the group consisting of lysostaphin, mutanolysin β-N-acetylglucosaminidase and a combination of mutanolysin and β-N-acetylglucosaminidase. These act on the bacterial peptidoglycan to release the capsular saccharide for use with the invention but also lead to release of the group-specific carbohydrate antigen. In an embodiment, the bacterial cells are treated by a type II phosphodiesterase (PDE2).

Optionally, after polysaccharide release, the enzyme(s) is/are deactivated. A suitable method for deactivation is for example heat treatment or acidic treatment.

In an embodiment, the bacterial cells (e.g. in suspension in their original culture medium, in the form of a wet cell paste, in a dried form or resuspended in an aqueous medium after centrifugation) are autoclaved such that the polysaccharide is released.

In a further embodiment, the bacterial cells (e.g. in suspension in their original culture medium, in the form of a wet cell paste, in a dried form or resuspended in an aqueous medium after centrifugation) are chemically treated such that the polysaccharide is released. In such an embodiment, the chemical treatment can be for example hydrolysis using base or acid (see e.g. WO2007084856).

In an embodiment, the bacterial cells chemical treatment is base extraction (e.g., using sodium hydroxide). Base extraction can cleave the phosphodiester linkage between the capsular saccharide and the peptidoglycan backbone. In an embodiment, the base is selected from the group consisting of NaOH, KOH, LiOH, NaHC03, Na2C03, KzC03, KCN, Et3N, NH3, HzN2H2, NaH, NaOMe, NaOEt and KOtBu. After base treatment, the reaction mixture may be neutralised. This may be achieved by the addition of an acid. In an embodiment, after base treatment, the reaction mixture is neutralised by an acid selected from the group consisting of HCl, H₃PO₄, citric acid, acetic acid, nitrous acid, and sulfuric acid.

In an embodiment, the bacterial cells chemical treatment is acid treatment (e.g., sulfuric acid). In an embodiment, the acid is selected from the group consisting of HCl, H₃PO₄, citric acid, acetic acid, nitrous acid, and sulfuric acid. Following acid treatment, the reaction mixture may be neutralised. This may be achieved by the addition of a base. In an embodiment, after acid treatment, the reaction mixture is neutralised by a base selected from the group consisting of NaOH, KOH, LiOH, NaHC03, Na2C03, KzC03, KCN, Et3N, NH3, HzN2H2, NaH, NaOMe, NaOEt and KOtBu.

1.2 Flocculation

The methods of the invention comprise a flocculation step. The inventors have found that the process results in a purified polysaccharide with low contamination. The inventor's process can be quick and simple.

Therefore in the method of the invention, the solution obtained by any of the method of section 1.1 above is treated by flocculation.

In the present invention, the term “flocculation” refers to a process wherein colloids come out of suspension in the form of floc or flake due to the addition of a flocculating agent.

The flocculation step comprises adding a “flocculating agent” to a solution comprising bacterial polysaccharides together with contaminants. In an embodiment, the contaminants comprise bacterial cell debris, bacterial cell proteins and nucleic acids. In an embodiment, the contaminants comprise bacterial cell proteins and nucleic acids. As it will be further disclosed herebelow, the flocculation step may further include adjustment of the pH, either before or after the addition of the flocculating agent. In particular the solution may be acidified.

Furthermore, the addition of the flocculating agent and/or the adjustment of the pH may be performed at a temperature adjusted to a desirable level.

These steps can be performed in any order:
addition of the flocculating agent followed by adjustment of the pH followed by adjustment of the temperature or;
addition of the flocculating agent followed by adjustment of the temperature followed by adjustment of the pH or;
adjustment of the pH followed by addition of the flocculating agent followed by adjustment of the temperature or;
adjustment of the pH followed by adjustment of the temperature followed by addition of the flocculating agent or;
adjustment of the temperature followed by addition of the flocculating agent followed by adjustment of the pH or;
adjustment of the temperature followed by adjustment of the pH followed by addition of the flocculating agent.

Furthermore, following the addition of the flocculating agent and/or the adjustment of the pH, the solution may be hold for some time to allow settling of the flocs prior to downstream processing.

In the present invention a “flocculating agent” refers to an agent being capable of allowing, in a solution comprising a polysaccharide of interest together with contaminants, promoting flocculation by causing colloids and other suspended particles to aggregate in the form of floc or flake, while the polysaccharide of interest significantly stays in solution.

In an embodiment of the present invention, the flocculating agent comprises a multivalent cation. In an embodiment, the flocculating agent is a multivalent cation. In a preferred embodiment said multivalent cation is selected from the group consisting of aluminium, iron, calcium and magnesium. In an embodiment the flocculating agent is a mixture of at least two multivalent cations selected from the group consisting of aluminium, iron, calcium and magnesium. In an embodiment the flocculating agent is a mixture of at least three multivalent cations selected from the group consisting of aluminium, iron, calcium and magnesium. In an embodiment the flocculating agent is a mixture of four multivalent cations consisting of aluminium, iron, calcium and magnesium.

In an embodiment, the flocculating agent comprises an agent selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate. In an embodiment, the flocculating agent is selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate. In an embodiment, the flocculating agent is polyethylenimine (PEI). In an embodiment, the flocculating agent comprises alum. In an embodiment, the flocculating agent is alum. In an embodiment, the flocculating agent comprises potassium alum. In an embodiment, the flocculating agent is potassium alum. In an embodiment, the flocculating agent comprises sodium alum. In an embodiment, the flocculating agent is sodium alum. In an embodiment, the flocculating agent comprises ammonium alum. In an embodiment, the flocculating agent is ammonium alum.

In an embodiment, the flocculating agent is a mixture of agents (e.g. two, three or four agents) selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate. In an embodiment, the flocculating agent is selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.

In an embodiment, the flocculating agent is a mixture of two agents selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate. In an embodiment, the flocculating agent is a mixture of at least three agents selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.

In an embodiment, the flocculating agent comprises an agent selected from the group consisting of chitosan, isinglass, moringa oleifera seeds (Horseradish Tree), gelatin, strychnos potatorum seeds (Nirmali nut tree), guar gum and alginates (e.g. brown seaweed extracts). In an embodiment, the flocculating agent is selected from the group consisting of chitosan, isinglass, moringa oleifera seeds (Horseradish Tree), gelatin, strychnos potatorum seeds (Nirmali nut tree), guar gum and alginates (e.g. brown seaweed extracts).

The concentration of flocculating agent may depend on the agent(s) used, the polysaccharide of interest and the parameter of the flocculation step (e.g. temperature etc. . . . ).

In embodiments where the flocculating agent comprises or is alum, a concentration of flocculating agent of between about 0.1 and 20% (w/v) can be used. Preferably through a concentration of flocculating agent of between about 0.5 and 10% (w/v) is used. Even more preferably a concentration of flocculating agent of between about 1 and 5% (w/v) is used.

Any number within any of the above ranges is contemplated as an embodiment of the disclosure.

In an embodiment, a concentration of flocculating agent of about 0.1, about 0.25, about 0.5, about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10% (w/v) is used. In an embodiment, a concentration of flocculating agent of about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5 or about 20.0% (w/v) is used. In an embodiment, a concentration of flocculating agent of about 0.5, about 1.0, about 1.5, about 2.0, about 2.5, about 3.0, about 3.5, about 4.0, about 4.5 or about 5.0% (w/v) is used. In an embodiment, a concentration of flocculating agent of about 1.0, about 1.5, about 2.0, about 2.5, about 3.0, about 3.5 or about 4.0% (w/v) is used.

In some embodiments of the present invention, the flocculating agent is added over a certain period of time. In some embodiments of the present invention, the flocculating agent is added over a period of between a few seconds (e.g. 1 to 10 seconds) and about one month. In some embodiments the flocculating agent is added over a period of between about 2 seconds and about two weeks. In some embodiments of the present invention, the flocculating agent is added over a period of between about 1 minute and about one week. In some embodiments the flocculating agent is added over a period of between about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours and about two days.

Therefore in certain embodiments, the flocculating agent is added over a period of between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.

Preferably the flocculating agent is added over a period of between about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.

In certain embodiments the flocculating agent is added over a period of between about 15 minutes and about 3 hours. In certain embodiments the flocculating agent is added over a period of between about 30 minutes and about 120 minutes.

Any number within any of the above ranges is contemplated as an embodiment of the disclosure.

The flocculating agent may be added over a period of about 2 seconds, about 10 seconds, about 30 seconds, about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 170 minutes, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, about 48 hours, about 3 days, about 4 days, about 5 days, about 6 days, about 7 days, about 8 days, about 9 days, about 10 days, about 11 days, about 12 days, about 13 days, about 14 days or about 15 days.

In an embodiment, the flocculating agent is added without agitation. In another embodiment, the flocculating agent is added under agitation. In another embodiment, the flocculating agent is added under gentle agitation. In another embodiment, the flocculating agent is added under vigorous agitation.

The inventors have further surprisingly noted that the flocculation is improved when performed at an acidic pH.

Therefore in an embodiment of the present invention, the flocculation step is performed at a pH below 7.0, 6.0, 5.0 or 4.0. In a particular embodiment of the present invention, the flocculation step is performed at a pH between 7.0 and 1.0. In an embodiment, the flocculation step is performed at a pH between 5.5 and 2.5, 5.0 and 2.5, 4.5 and 2.5, 4.0 and 2.5, 5.5 and 3.0, 5.0 and 3.0, 4.5 and 3.0, 4.0 and 3.0, 5.5 and 3.5, 5.0 and 3.5, 4.5 and 3.5 or 4.0 and 3.5. In an embodiment, the flocculation step is performed at a pH of about 5.5, about 5.0, about 4.5, about 4.0, about 3.5, about 3.0, about 2.5, about 2.0, about 1.5 or about 1.0. In an embodiment, the flocculation step is performed at a pH of about 4.0, about 3.5, about 3.0 or about 2.5. In an embodiment, the flocculation step is performed at a pH of about 3.5.

Any number within any of the above ranges is contemplated as an embodiment of the disclosure.

In an embodiment, said acidic pH is obtained by acidifying the solution obtained by any of the method of section 1.1 above or further clarified as disclosed at section 1.2 with an acid. In an embodiment said acid is selected from the group consisting of HCl, H₃PO₄, citric acid, acetic acid, nitrous acid, and sulfuric acid. In an embodiment said acid is an amino acid. In an embodiment said acid is an amino acid selected from the group consisting of glycine, alanine and glutamate. In an embodiment said acid is HCl (hydrochloric acid). In an embodiment said acid is sulfuric acid.

In an embodiment, the acid is added is without agitation. Preferably, the acid is added is under agitation. In an embodiment, the acid is added under gentle agitation. In an embodiment, the acid is added under vigorous agitation.

In some embodiments of the present invention, following the addition of the flocculating agent (and the optional acidification), the solution is hold for some time to allow settling of the flocs prior to downstream processing.

In some embodiments of the present invention, the flocculation step is performed with a settling time of between a few seconds (e.g. 2 to 10 seconds) to about 1 minute.

Preferably the settling time is at least about 2, at least about 3, at least about 4, at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 115, at least about 120, at least about 125, at least about 130, at least about 135, at least about 140, at least about 145, at least about 150, at least about 155 or at least about 160 minutes. Preferably the settling time is less than a week, however the settling time maybe longer.

Therefore in certain embodiments, the settling time is between about 1, about 2, about 3, about 4, about 5, about 6, about 7, about 8, about 9, about 10, about 15, about 20, about 25, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380, about 1440 minute(s), about two days, about three days, about four days, about five days or about six days and 1 week.

In some embodiments of the present invention, the settling time is between a few seconds (e.g. 1 to 10 seconds) and about one month. In some embodiments the settling time is between about 2 seconds and about two weeks. In some embodiments of the present invention, the settling time is between about 1 minute and about one week. In some embodiments the settling time is between about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours and about two days.

Therefore in certain embodiments, the settling time is between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.

Preferably the settling time is between about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.

In certain embodiments the settling time is between about 15 minutes and about 3 hours. In certain embodiments the settling time is between about 30 minutes and about 120 minutes.

Any number within any of the above ranges is contemplated as an embodiment of the disclosure.

In certain embodiments the settling time is about 2 seconds, about 10 seconds, about 30 seconds, about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 170 minutes, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, about 48 hours, about 3 days, about 4 days, about 5 days, about 6 days, about 7 days, about 8 days, about 9 days, about 10 days, about 11 days, about 12 days, about 13 days, about 14 days or about 15 days.

Preferably the settling time is between about 5, about 10, about 15, about 20, about 25, about 30, about 60, about 90, about 120, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380 or about 1440 minute(s) and two days. In certain embodiments the settling time is between about 5 minutes and about one day. In certain embodiments the settling time is between about 5 minutes and about 120 minutes.

The settling time may be about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes or about 160 minutes.

Any number within any of the above ranges is contemplated as an embodiment of the disclosure.

In an embodiment, the optional settling step is conducted without agitation. In an embodiment, the optional settling step is conducted under agitation. In another embodiment, the optional settling step is conducted under gentle agitation. In another embodiment, the optional settling step is conducted under vigorous agitation.

In an embodiment of the present invention, the addition of the flocculating agent, the settling of the solution and/or the adjustment of the pH is performed at a temperature between about 4° C. and about 30° C. In an embodiment, the addition of the flocculating agent, the settling of the solution and/or the adjustment of the pH is performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C. In an embodiment, the addition of the flocculating agent, the settling of the solution and/or the adjustment of the pH is performed at a temperature of about 20° C. The inventors have surprisingly noted that the flocculation can be further improved when performed at elevated temperature. Therefore in a particular embodiment of the present invention, the addition of the flocculating agent, the settling of the solution and/or the adjustment of the pH is performed at temperature between about 30° C. to about 95° C. In an embodiment, the addition of the flocculating agent, the settling of the solution and/or the adjustment of the pH is performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C. In an embodiment, the addition of the flocculating agent, the settling of the solution and/or the adjustment of the pH is performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the addition of the flocculating agent, the settling of the solution and/or the adjustment of the pH is performed at a temperature of about 50° C. Any number within any of the above ranges is contemplated as an embodiment of the disclosure.

In an embodiment, the addition of the flocculating agent is performed at any of the above mentioned temperatures.

In an embodiment, the settling of the solution after the addition of the flocculating agent is performed at any of the above mentioned temperatures.

In an embodiment, the adjustment of the pH is performed at any of the above mentioned temperatures.

In an embodiment, the addition of the flocculating agent and the settling of the solution after the addition of the flocculating agent are performed at any of the above mentioned temperatures.

In an embodiment, the addition of the flocculating agent and the adjustment of the pH are performed at any of the above mentioned temperatures.

In an embodiment, the addition of the flocculating, the settling of the solution after the addition of the flocculating agent and the adjustment of the pH are performed at any of the above mentioned temperatures.

In an embodiment, the flocculation step comprises adding a flocculating agent (as disclosed above) without pH adjustment.

In an embodiment, the flocculation step comprises adding a flocculating agent and settling the solution (as disclosed above), without pH adjustment.

In an embodiment, the flocculation step comprises adding a flocculating agent, adjusting the pH and settling the solution (as disclosed above). In an embodiment, the flocculating agent is added before adjusting the pH. In another embodiment, the pH is adjusted before adding the flocculating agent.

In an embodiment, the flocculation step comprises adding a flocculating agent, settling the solution and adjusting the pH (as disclosed above). In an embodiment, the addition of flocculating agent and settling of the solution is conducted before adjusting the pH. In another embodiment, the pH is adjusted before adding the flocculating agent and settling the solution. In an embodiment, the addition of the flocculating agent and adjusting the pH is conducted before settling the solution. In another embodiment, the pH is adjusted before adding the flocculating agent and settling the solution.

In an embodiment, the flocculation step comprises adding a flocculating agent, adjusting the pH and adjustment of the temperature (as disclosed above).

These steps can be performed in any order:
addition of the flocculating agent followed by adjustment of the pH followed by adjustment of the temperature or;
addition of the flocculating agent followed by adjustment of the temperature followed by adjustment of the pH or;
adjustment of the pH followed by addition of the flocculating agent followed by adjustment of the temperature or;
adjustment of the pH followed by adjustment of the temperature followed by addition of the flocculating agent or;
adjustment of the temperature followed by addition of the flocculating agent followed by adjustment of the pH or;
adjustment of the temperature followed by adjustment of the pH followed by addition of the flocculating agent.

Furthermore, following the addition of the flocculating agent and/or the adjustment of the pH, the solution may be hold for some time to allow settling of the flocs prior to downstream processing.

1.3 Solid/Liquid Separation

The flocculated material can be separated from the polysaccharide of interest by any suitable solid/liquid separation method.

Therefore in an embodiment of the present invention, after flocculation, the suspension (as obtained at section 1.2 above) is clarified by decantation, sedimentation, filtration or centrifugation. In an embodiment the polysaccharide-containing solution is then collected for storage and/or additional processing.

In an embodiment of the present invention, after flocculation, the suspension (as obtained at section 1.2 above) is clarified by decantation. Decanters are used to separate liquids where there is a sufficient difference in density between the liquids for the floc to settle. In an operating decanter there will be three distinct zones: clear heavy liquid, separating dispersed liquid (the dispersion zone), and clear light liquid. To produce a clean solution, a small amount of solution must generally be left in the container. Decanters can be designed for continuous operation.

In an embodiment of the present invention, after flocculation, the suspension (as obtained at section 1.2 above) is clarified by sedimentation (settling). Sedimentation is the separation of suspended solid particles from a liquid mixture by gravity settling into a clear fluid and a slurry of higher solids content. Sedimentation can be done in a thickener, in a clarifier or in a classifier. Since thickening and clarification are relatively cheap processes when used for the treatment of large volumes of liquid, they can be used for pre-concentration of feeds to filtering.

In an embodiment of the present invention, after flocculation, the suspension (as obtained at section 1.2 above) is clarified by centrifugation. In an embodiment said centrifugation is continuous centrifugation. In an embodiment said centrifugation is bucket centrifugation. In an embodiment the polysaccharide-containing supernatant is then collected for storage and/or additional processing.

In some embodiments the suspension is centrifuged at about 1,000 g about 2,000 g, about 3,000 g, about 4,000 g, about 5,000 g, about 6,000 g, about 8,000 g, about 9,000 g, about 10,000 g, about 11,000 g, about 12,000 g, about 13,000 g, about 14,000 g, about 15,000 g, about 16,000 g, about 17,000 g, about 18,000 g, about 19,000 g, about 20,000 g, about 25,000 g, about 30,000 g, about 35,000 g, about 40,000 g, about 50,000 g, about 60,000 g, about 70,000 g, about 80,000 g, about 90,000 g, about 100,000 g, about 120,000 g, about 140,000 g, about 160,000 g or about 180,000 g. In some embodiments the suspension is centrifuged at about 8,000 g, about 9,000 g, about 10,000 g, about 11,000 g, about 12,000 g, about 13,000 g, about 14,000 g, about 15,000 g, about 16,000 g, about 17,000 g, about 18,000 g, about 19,000 g, about 20,000 g or about 25,000 g.

In some embodiments the suspension is centrifuged between about 5,000 g and about 25,000 g. In some embodiments the suspension is centrifuged between about 8,000 g and about 20,000 g. In some embodiments the suspension is centrifuged between about 10,000 g and about 15,000 g. In some embodiments the suspension is centrifuged between about 10,000 g and about 12,000 g.

Any number within any of the above ranges is contemplated as an embodiment of the disclosure.

In some embodiments the suspension is centrifuged during at least 2, at least 3, at least 4, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155 or at least 160 minutes. Preferably the centrifugation time is less than 24 hours.

Therefore in certain embodiments, the suspension is centrifuged during between about 5, about 10, about 15, about 20, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320 or about 1380 minutes and 1440 minutes.

Preferably the suspension is centrifuged during between about 5, about 10, about 15, about 20, about 25, about 30, about 60, about 90, about 120, about 180, about 240, about 300, about 360, about 420, about 480 or about 540 minutes and about 600 minutes. In certain embodiments the suspension is centrifuged during between about 5 minutes and about 3 hours. In certain the suspension is centrifuged during between about 5 minutes and about 120 minutes.

The suspension may be centrifuged during between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes or about 155 minutes and about 160 minutes.

The suspension may be centrifuged during between about 10 minutes, about 15 minutes, about 20 minutes, about 25 minutes, about 30 minutes, about 35 minutes, about 40 minutes, about 45 minutes, about 50 minutes or about 55 minutes and about 60 minutes.

The suspension may be centrifuged during about 5, about 10, about 15, about 20, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380 minutes or about 1440 minutes.

The suspension may be centrifuged during about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes or about 160 minutes.

The suspension may be centrifuged during between about 10 minutes, about 15 minutes, about 20 minutes, about 25 minutes, about 30 minutes, about 35 minutes, about 40 minutes, about 45 minutes, about 50 minutes, about 55 minutes or about 60 minutes.

Any number within any of the above ranges is contemplated as an embodiment of the disclosure.

In an embodiment of the present invention, centrifugation is continuous centrifugation. In said embodiment, the feed rate can be of between of 50-5000 ml/min, 100-4000 ml/min, 150-3000 ml/min, 200-2500 ml/min, 250-2000 ml/min, 300-1500 ml/min, 300-1000 ml/min, 200-1000 ml/min, 200-1500 ml/min, 400-1500 ml/min, 500-1500 ml/min, 500-1000 ml/min, 500-2000 ml/min, 500-2500 ml/min or 1000-2500 ml/min.

In an embodiment, the feed rate can be of about 10, about 25, about 50, about 75, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1650 about 1700, about 1800, about 1900, about 2000, about 2100, about 2200, about 2300, about 2400, about 2500, about 2600, about 2700, about 2800, about 2900, about 3000, about 3250, about 3500, about 3750 about 4000, about 4250, about 4500 or about 5000 ml/min.

In an embodiment of the present invention, after flocculation, the suspension (as obtained at section 1.2 above) is clarified by filtration. In filtration, suspended solid particles in a liquid are removed by passing the mixture through a porous medium that retains particles and passes the clear filtrate. Filtration is performed on screens by gravity or on filters by vacuum, pressure or centrifugation. The solid can be retained on the surface of the filter medium, which is cake filtration, or captured within the filter medium, which is depth filtration. In an embodiment, after flocculation, the suspension (as obtained at section 1.2 above) is clarified by microfiltration. In an embodiment, microfiltration is tangential microfiltration. In another embodiment, microfiltration is dead-end filtration (perpendicular filtration). In an embodiment, microfiltration is dead-end filtration wherein diatomaceous earth (DE), also known as DE diatomite, is used as a filter aid to facilitate and enhance the efficiency of the solid/liquid separation. Therefore in an embodiment, after flocculation, the suspension (as obtained at section 1.2 above) is clarified by dead-end microfiltration comprising diatomaceous earth (DE). DE can be impregnated (or incorporated) into to the dead-end filter as an integral part of the depth filter.

In another format, the DE can be added to the flocculated solution (as obtained after section 1.2) in powder form. In the later case, the DE treated flocculated solution can be further clarified by depth filtration.

In an embodiment, the solution is treated by a microfiltration step wherein the filter has a nominal retention range of between about 0.01-2 micron, about 0.05-2 micron, about 0.1-2 micron, about 0.2-2 micron, about 0.3-2 micron, about 0.4-2 micron, about 0.45-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, or about 1.75-2 micron.

In an embodiment, the solution is treated by a microfiltration step wherein the filter has a nominal retention range of between about 0.01-1 micron, about 0.05-1 micron, about 0.1-1 micron, about 0.2-1 micron, about 0.3-1 micron, about 0.4-1 micron, about 0.45-1 micron, about 0.5-1 micron, about 0.6-1 micron, about 0.7-1 micron, about 0.8-1 micron or about 0.9-1 micron.

Any number within any of the above ranges is contemplated as an embodiment of the disclosure.

In an embodiment, the solution is treated by a microfiltration step wherein the filter has a nominal retention rating of about 0.01, about 0.05, about 0.1, about 0.2, about 0.3, about 0.4, about 0.45, about 0.5, about 0.6, about 0.7, about 0.8, about 0.9, about 1, about 1.1, about 1.2, about 1.3, about 1.4, about 1.5, about 1.6, about 1.7, about 1.8, about 1.9 or about 2 micron.

In an embodiment, the solution is treated by a microfiltration step wherein the filter has a nominal retention rating of about 0.45 micron.

In an embodiment, the solution is treated by a microfiltration step wherein the filter has a filter capacity of between 100-5000 L/m², 200-5000 L/m², 300-5000 L/m², 400-5000 L/m², 500-5000 L/m², 750-5000 L/m², 1000-5000 L/m², 1500-5000 L/m², 2000-5000 L/m², 3000-5000 L/m² or 4000-5000 L/m².

In an embodiment, the solution is treated by a microfiltration step wherein the filter has a filter capacity of between 100-2500 L/m², 200-2500 L/m², 300-2500 L/m², 400-2500 L/m², 500-2500 L/m², 750-2500 L/m², 1000-2500 L/m², 1500-2500 L/m² or 2000-2500 L/m².

In an embodiment, the solution is treated by a microfiltration step wherein the filter has a filter capacity of between 100-1500 L/m², 200-1500 L/m², 300-1500 L/m², 400-1500 L/m², 500-1500 L/m², 750-1500 L/m² or 1000-1500 L/m².

In an embodiment, the solution is treated by a microfiltration step wherein the filter has a filter capacity of between 100-1250 L/m², 200-1250 L/m², 300-1250 L/m², 400-1250 L/m², 500-1250 L/m², 750-1250 L/m² or 1000-1250 L/m².

In an embodiment, the solution is treated by a microfiltration step wherein the filter has a filter capacity of between 100-1000 L/m², 200-1000 L/m², 300-1000 L/m², 400-1000 L/m², 500-1000 L/m² or 750-1000 L/m².

In an embodiment, the solution is treated by a microfiltration step wherein the filter has a filter capacity of between 100-750 L/m², 200-750 L/m², 300-750 L/m², 400-750 L/m² or 500-750 L/m².

In an embodiment, the solution is treated by a microfiltration step wherein the filter has a filter capacity of between 100-600 L/m², 200-600 L/m², 300-600 L/m², 400-600 L/m² or 400-600 L/m².

In an embodiment, the solution is treated by a microfiltration step wherein the filter has a filter capacity of between 100-500 L/m², 200-500 L/m², 300-500 L/m² or 400-500 L/m².

Any number within any of the above ranges is contemplated as an embodiment of the disclosure.

In an embodiment, the solution is treated by a microfiltration step wherein the filter has a filter capacity of about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450 or about 2500 L/m².

The solid/liquid separation methods described above can be used in a standalone format or in combination of two in any order, or in combination of three in any order.

1.4 Filtration (e.g. Depth Filtration)

Once the solution has been treated by the flocculation step of section 1.2 above and/or by the solid/liquid separation step of section 1.3 above, the polysaccharide containing solution (e.g. the supernatant) can optionally be further clarified.

In an embodiment, the solution is filtrated, thereby producing a further clarified solution.

In an embodiment, the filtration is applied directly to the solution obtained by any of the method of section 1.2 above. In an embodiment, the filtration is applied to the solution further clarified by the solid/liquid separation step as described at section 1.3 above.

In an embodiment, the solution is treated by a filtration step selected from the group consisting of depth filtration, filtration through activated carbon, size filtration, diafiltration and ultrafiltration. In an embodiment, the solution is treated by a diafiltration step, particularly by tangential flow filtration. In an embodiment, the solution is treated by a depth filtration step.

Depth filters use a porous filtration medium to retain particles throughout the medium, rather than just on the surface of the medium. Due to the tortuous and channel-like nature of the filtration medium, the particles are retained throughout the medium within its structure, as opposed to on the surface.

In an embodiment, the solution is treated by a depth filtration step wherein the depth filter design is selected from the group consisting of cassettes, cartridges, deep bed (e.g. sand filter) and lenticular filters.

In an embodiment, the solution is treated by a depth filtration step wherein the depth filter has a nominal retention range of between about 0.01-100 micron, about 0.05-100 micron, about 0.1-100 micron, about 0.2-100 micron, about 0.3-100 micron, about 0.4-100 micron, about 0.5-100 micron, about 0.6-100 micron, about 0.7-100 micron, about 0.8-100 micron, about 0.9-100 micron, about 1-100 micron, about 1.25-100 micron, about 1.5-100 micron, about 1.75-100 micron, about 2-100 micron, about 3-100 micron, about 4-100 micron, about 5-100 micron, about 6-100 micron, about 7-100 micron, about 8-100 micron, about 9-100 micron, about 10-100 micron, about 15-100 micron, about 20-100 micron, about 25-100 micron, about 30-100 micron, about 40-100 micron, about 50-100 micron or about 75-100 micron.

In an embodiment, the solution is treated by a depth filtration step wherein the depth filter has a nominal retention range of between about 0.01-75 micron, about 0.05-75 micron, about 0.1-75 micron, about 0.2-75 micron, about 0.3-75 micron, about 0.4-75 micron, about 0.5-75 micron, about 0.6-75 micron, about 0.7-75 micron, about 0.8-75 micron, about 0.9-75 micron, about 1-75 micron, about 1.25-75 micron, about 1.5-75 micron, about 1.75-75 micron, about 2-75 micron, about 3-75 micron, about 4-75 micron, about 5-75 micron, about 6-75 micron, about 7-75 micron, about 8-75 micron, about 9-75 micron, about 10-75 micron, about 15-75 micron, about 20-75 micron, about 25-75 micron, about 30-75 micron, about 40-75 micron or about 50-75 micron.

In an embodiment, the solution is treated by a depth filtration step wherein the depth filter has a nominal retention range of between about 0.01-50 micron, about 0.05-50 micron, about 0.1-50 micron, about 0.2-50 micron, about 0.3-50 micron, about 0.4-50 micron, about 0.5-50 micron, about 0.6-50 micron, about 0.7-50 micron, about 0.8-50 micron, about 0.9-50 micron, about 1-50 micron, about 1.25-50 micron, about 1.5-50 micron, about 1.75-50 micron, about 2-50 micron, about 3-50 micron, about 4-50 micron, about 5-50 micron, about 6-50 micron, about 7-50 micron, about 8-50 micron, about 9-50 micron, about 10-50 micron, about 15-50 micron, about 20-50 micron, about 25-50 micron, about 30-50 micron, about 40-50 micron or about 50-50 micron.

In an embodiment, the solution is treated by a depth filtration step wherein the depth filter has a nominal retention range of between about 0.01-25 micron, about 0.05-25 micron, about 0.1-25 micron, about 0.2-25 micron, about 0.3-25 micron, about 0.4-25 micron, about 0.5-25 micron, about 0.6-25 micron, about 0.7-25 micron, about 0.8-25 micron, about 0.9-25 micron, about 1-25 micron, about 1.25-25 micron, about 1.5-25 micron, about 1.75-25 micron, about 2-25 micron, about 3-25 micron, about 4-25 micron, about 5-25 micron, about 6-25 micron, about 7-25 micron, about 8-25 micron, about 9-25 micron, about 10-25 micron, about 15-25 micron or about 20-25 micron.

In an embodiment, the solution is treated by a depth filtration step wherein the depth filter has a nominal retention range of between about 0.01-10 micron, about 0.05-10 micron, about 0.1-10 micron, about 0.2-10 micron, about 0.3-10 micron, about 0.4-10 micron, about 0.5-10 micron, about 0.6-10 micron, about 0.7-10 micron, about 0.8-10 micron, about 0.9-10 micron, about 1-10 micron, about 1.25-10 micron, about 1.5-10 micron, about 1.75-10 micron, about 2-10 micron, about 3-10 micron, about 4-10 micron, about 5-10 micron, about 6-10 micron, about 7-10 micron, about 8-10 micron or about 9-10 micron.

In an embodiment, the solution is treated by a depth filtration step wherein the depth filter has a nominal retention range of between about 0.01-8 micron, about 0.05-8 micron, about 0.1-8 micron, about 0.2-8 micron, about 0.3-8 micron, about 0.4-8 micron, about 0.5-8 micron, about 0.6-8 micron, about 0.7-8 micron, about 0.8-8 micron, about 0.9-8 micron, about 1-8 micron, about 1.25-8 micron, about 1.5-8 micron, about 1.75-8 micron, about 2-8 micron, about 3-8 micron, about 4-8 micron, about 5-8 micron, about 6-8 micron or about 7-8 micron.

In an embodiment, the solution is treated by a depth filtration step wherein the depth filter has a nominal retention range of between about 0.01-5 micron, about 0.05-5 micron, about 0.1-5 micron, about 0.2-5 micron, about 0.3-5 micron, about 0.4-5 micron, about 0.5-5 micron, about 0.6-5 micron, about 0.7-5 micron, about 0.8-5 micron, about 0.9-5 micron, about 1-5 micron, about 1.25-5 micron, about 1.5-5 micron, about 1.75-5 micron, about 2-5 micron, about 3-5 micron or about 4-5 micron.

In an embodiment, the solution is treated by a depth filtration step wherein the depth filter has a nominal retention range of between about 0.01-2 micron, about 0.05-2 micron, about 0.1-2 micron, about 0.2-2 micron, about 0.3-2 micron, about 0.4-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, about 1.75-2 micron, about 2-2 micron, about 3-2 micron or about 4-2 micron.

In an embodiment, the solution is treated by a depth filtration step wherein the depth filter has a nominal retention range of between about 0.01-1 micron, about 0.05-1 micron, about 0.1-1 micron, about 0.2-1 micron, about 0.3-1 micron, about 0.4-1 micron, about 0.5-1 micron, about 0.6-1 micron, about 0.7-1 micron, about 0.8-1 micron or about 0.9-1 micron.

In an embodiment, the solution is treated by a depth filtration step wherein the depth filter has a nominal retention range of between about 0.05-50 micron, 0.1-25 micron 0.2-10, micron 0.1-10 micron, 0.2-5 micron or 0.25-1 micron.

Any number within any of the above ranges is contemplated as an embodiment of the disclosure.

In an embodiment, the solution is treated by a depth filtration step wherein the depth filter has a filter capacity of 1-2500 L/m², 5-2500 L/m², 10-2500 L/m², 25-2500 L/m², 50-2500 L/m², 75-2500 L/m², 100-2500 L/m², 150-2500 L/m², 200-2500 L/m², 300-2500 L/m², 400-2500 L/m², 500-2500 L/m², 750-2500 L/m², 1000-2500 L/m², 1500-2500 L/m² or 2000-2500 L/m².

In an embodiment, the solution is treated by a depth filtration step wherein the depth filter has a filter capacity of 1-1000 L/m², 5-1000 L/m², 10-1000 L/m², 25-1000 L/m², 50-1000 L/m², 75-1000 L/m², 100-1000 L/m², 150-1000 L/m², 200-1000 L/m², 300-1000 L/m², 400-1000 L/m², 500-1000 L/m² or 750-1000 L/m².

In an embodiment, the solution is treated by a depth filtration step wherein the depth filter has a filter capacity of 1-750 L/m², 5-750 L/m², 10-750 L/m², 25-750 L/m², 50-750 L/m², 75-750 L/m², 100-750 L/m², 150-750 L/m², 200-750 L/m², 300-750 L/m², 400-750 L/m² or 500-750 L/m².

In an embodiment, the solution is treated by a depth filtration step wherein the depth filter has a filter capacity of 1-500 L/m², 5-500 L/m², 10-500 L/m², 25-500 L/m², 50-500 L/m², 75-500 L/m², 100-500 L/m², 150-500 L/m², 200-500 L/m², 300-500 L/m² or 400-500 L/m².

In an embodiment, the solution is treated by a depth filtration step wherein the depth filter has a filter capacity of 1-400 L/m², 5-400 L/m², 10-400 L/m², 25-400 L/m², 50-400 L/m², 75-400 L/m², 100-400 L/m², 150-400 L/m², 200-400 L/m² or 300-400 L/m².

In an embodiment, the solution is treated by a depth filtration step wherein the depth filter has a filter capacity of 1-300 L/m², 5-300 L/m², 10-300 L/m², 25-300 L/m², 50-300 L/m², 75-300 L/m², 100-300 L/m², 150-300 L/m² or 200-300 L/m².

In an embodiment, the solution is treated by a depth filtration step wherein the depth filter has a filter capacity of 1-200 L/m², 5-200 L/m², 10-200 L/m², 25-200 L/m², 50-200 L/m², 75-200 L/m², 100-200 L/m² or 150-200 L/m².

In an embodiment, the solution is treated by a depth filtration step wherein the depth filter has a filter capacity of 1-100 L/m², 5-100 L/m², 10-100 L/m², 25-100 L/m², 50-100 L/m² or 75-100 L/m².

In an embodiment, the solution is treated by a depth filtration step wherein the depth filter has a filter capacity of 1-50 L/m², 5-50 L/m², 10-50 L/m² or 25-50 L/m². Any whole number integer within any of the above ranges is contemplated as an embodiment of the disclosure.

In an embodiment, the solution is treated by a depth filtration step wherein the feed rate is between 1-1000 LMH (liters/m²/hour), 10-1000 LMH, 25-1000 LMH, 50-1000 LMH, 100-1000 LMH, 125-1000 LMH, 150-1000 LMH, 200-1000 LMH, 250-1000 LMH, 300-1000 LMH, 400-1000 LMH, 500-1000 LMH, 600-1000 LMH, 700-1000 LMH, 800-1000 LMH or 900-1000 LMH.

In an embodiment, the solution is treated by a depth filtration step wherein the feed rate is between 1-500 LMH, 10-500 LMH, 25-500 LMH, 50-500 LMH, 100-500 LMH, 125-500 LMH, 150-500 LMH, 200-500 LMH, 250-500 LMH, 300-500 LMH or 400-500 LMH.

In an embodiment, the solution is treated by a depth filtration step wherein the feed rate is between 1-400 LMH, 10-400 LMH, 25-400 LMH, 50-400 LMH, 100-400 LMH, 125-400 LMH, 150-400 LMH, 200-400 LMH, 250-400 LMH or 300-400 LMH.

In an embodiment, the solution is treated by a depth filtration step wherein the feed rate is between 1-250 LMH, 10-250 LMH, 25-250 LMH, 50-250 LMH, 100-250 LMH, 125-250 LMH, 150-250 LMH or 200-250 LMH.

Any number within any of the above ranges is contemplated as an embodiment of the disclosure.

In an embodiment, the solution is treated by a depth filtration step wherein the feed rate is about 1, about 2, about 5, about 10, about 25, about 50, about 60, about 70, about 80, about 90, about 100, about 110, about 120, about 130, about 140, about 150, about 160, about 170, about 180, about 190, about 200, about 210, about 220, about 230, about 240 about 250, about 260, about 270, about 280, about 290, about 300, about 310, about 320, about 330, about 340, about 350, about 360, about 370, about 380, about 390, about 400, about 425, about 450, about 475, about 500, about 525, about 550, about 575, about 600, about 650, about 700, about 750, about 800, about 850, about 900, about 950 or about 1000 LMH.

1.5 Optional Further Filtration

Once the solution has been treated by the filtration step of section 1.4 above, the solution obtained (i.e. the filtrate) can optionally be further clarified.

In an embodiment, the solution is subjected to microfiltration. In an embodiment, microfiltration is dead-end filtration (perpendicular filtration). In an embodiment, microfiltration is tangential microfiltration.

In an embodiment, the solution is treated by a microfiltration step wherein the filter has a nominal retention range of between about 0.01-2 micron, about 0.05-2 micron, about 0.1-2 micron, about 0.2-2 micron, about 0.3-2 micron, about 0.4-2 micron, about 0.45-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, or about 1.75-2 micron.

In an embodiment, the solution is treated by a depth filtration step wherein the filter has a nominal retention range of between about 0.01-1 micron, about 0.05-1 micron, about 0.1-1 micron, about 0.2-1 micron, about 0.3-1 micron, about 0.4-1 micron, about 0.45-1 micron, about 0.5-1 micron, about 0.6-1 micron, about 0.7-1 micron, about 0.8-1 micron or about 0.9-1 micron.

Any number within any of the above ranges is contemplated as an embodiment of the disclosure.

In an embodiment, the solution is treated by a microfiltration step wherein the filter has a nominal retention rating of about 0.01, about 0.05, about 0.1, about 0.2, about 0.3, about 0.4, about 0.45, about 0.5, about 0.6, about 0.7, about 0.8, about 0.9, about 1, about 1.1, about 1.2, about 1.3, about 1.4, about 1.5, about 1.6, about 1.7, about 1.8, about 1.9 or about 2 micron.

In an embodiment, the solution is treated by a microfiltration step wherein the filter has a nominal retention rating of about 0.45 micron.

In an embodiment, the solution is treated by a microfiltration step wherein the filter has a filter capacity of between 100-5000 L/m², 200-5000 L/m², 300-5000 L/m², 400-5000 L/m², 500-5000 L/m², 750-5000 L/m², 1000-5000 L/m², 1500-5000 L/m², 2000-5000 L/m², 3000-5000 L/m² or 4000-5000 L/m².

In an embodiment, the solution is treated by a microfiltration step wherein the filter has a filter capacity of between 100-2500 L/m², 200-2500 L/m², 300-2500 L/m², 400-2500 L/m², 500-2500 L/m², 750-2500 L/m², 1000-2500 L/m², 1500-2500 L/m² or 2000-2500 L/m².

In an embodiment, the solution is treated by a microfiltration step wherein the filter has a filter capacity of between 100-1500 L/m², 200-1500 L/m², 300-1500 L/m², 400-1500 L/m², 500-1500 L/m², 750-1500 L/m² or 1000-1500 L/m².

In an embodiment, the solution is treated by a microfiltration step wherein the filter has a filter capacity of between 100-1250 L/m², 200-1250 L/m², 300-1250 L/m², 400-1250 L/m², 500-1250 L/m², 750-1250 L/m² or 1000-1250 L/m².

In an embodiment, the solution is treated by a microfiltration step wherein the filter has a filter capacity of between 100-1000 L/m², 200-1000 L/m², 300-1000 L/m², 400-1000 L/m², 500-1000 L/m² or 750-1000 L/m².

In an embodiment, the solution is treated by a microfiltration step wherein the filter has a filter capacity of between 100-750 L/m², 200-750 L/m², 300-750 L/m², 400-750 L/m² or 500-750 L/m².

In an embodiment, the solution is treated by a microfiltration step wherein the filter has a filter capacity of between 100-600 L/m², 200-600 L/m², 300-600 L/m², 400-600 L/m² or 400-600 L/m².

In an embodiment, the solution is treated by a microfiltration step wherein the filter has a filter capacity of between 100-500 L/m², 200-500 L/m², 300-500 L/m² or 400-500 L/m².

Any number within any of the above ranges is contemplated as an embodiment of the disclosure.

In an embodiment, the solution is treated by a microfiltration step wherein the filter has a filter capacity of about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450 or about 2500 L/m².

1.6 Ultrafiltration and/or Diafiltration

Once the solution has been filtered by any of the method of section 1.4 above and/or by the filtration step of section 1.5 above, the solution obtained (i.e. the filtrate) can optionally be further clarified by Ultrafiltration and/or Dialfiltration.

Ultrafiltration (UF) is a process for concentrating a dilute product stream. UF separates molecules in solution based on the membrane pore size or molecular weight cutoff (MWCO).

In an embodiment of the present invention, the solution (e.g. the filtrate obtained at section 1.5 or 1.6 above) is treated by ultrafiltration.

In an embodiment, the solution is treated by ultrafiltration and the molecular weight cut off of the membrane is in the range of between about 5 kDa-1000 kDa. In an embodiment the molecular weight cut off of the membrane is in the range of between about 10 kDa-750 kDa. In an embodiment the molecular weight cut off of the membrane is in the range of between about 10 kDa-500 kDa. In an embodiment the molecular weight cut off of the membrane is in the range of between about 10 kDa-300 kDa. In an embodiment the molecular weight cut off of the membrane is in the range of between about 10 kDa-100 kDa. In an embodiment the molecular weight cut off of the membrane is in the range of between about 10 kDa-50 kDa. In an embodiment the molecular weight cut off of the membrane is in the range of between about 10 kDa-30 kDa. In an embodiment the molecular weight cut off of the membrane is in the range of between about 5 kDa-1000 kDa, about 10 kDa-1000 kDa about 20 kDa-1000 kDa, about 30 kDa-1000 kDa, about 40 kDa-1000 kDa, about 50 kDa-1000 kDa, about 75 kDa-1000 kDa, about 100 kDa-1000 kDa, about 150 kDa-1000 kDa, about 200 kDa-1000 kDa, about 300 kDa-1000 kDa, about 400 kDa-1000 kDa, about 500 kDa-1000 kDa or about 750 kDa-1000 kDa.

In an embodiment the molecular weight cut off of the membrane is in the range of between about 5 kDa-500 kDa, about 10 kDa-500 kDa, about 20 kDa-500 kDa, about 30 kDa-500 kDa, about 40 kDa-500 kDa, about 50 kDa-500 kDa, about 75 kDa-500 kDa, about 100 kDa-500 kDa, about 150 kDa-500 kDa, about 200 kDa-500 kDa, about 300 kDa-500 kDa or about 400 kDa-500 kDa.

In an embodiment the molecular weight cut off of the membrane is in the range of between about 5 kDa-300 kDa, about 10 kDa-300 kDa, about 20 kDa-300 kDa, about 30 kDa-300 kDa, about 40 kDa-300 kDa, about 50 kDa-300 kDa, about 75 kDa-300 kDa, about 100 kDa-300 kDa, about 150 kDa-300 kDa or about 200 kDa-300 kDa.

In an embodiment the molecular weight cut off of the membrane is in the range of between about 5 kDa-100 kDa, about 10 kDa-100 kDa, about 20 kDa-100 kDa, about 30 kDa-100 kDa, about 40 kDa-100 kDa, about 50 kDa-100 kDa or about 75 kDa-100 kDa.

In an embodiment the molecular weight cut off of the membrane is about 5 kDa, about 10 kDa, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 200 kDa, about 250 kDa, about 300 kDa, about 400 kDa, about 500 kDa, about 750 kDa or about 1000 kDa.

In an embodiment, the concentration factor of the ultrafiltration step is from about 1.5 to 10. In an embodiment, the concentration factor is from about 2 to 8. In an embodiment, the concentration factor is from about 2 to 5.

In an embodiment, the concentration factor is about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0. In an embodiment, the concentration factor is about 2, about 3, about 4, about 5, or about 6.

In an embodiment of the present invention, the solution (e.g. the filtrate obtained at section 1.4 or 1.5 above) is treated by diafiltration.

In an embodiment of the present invention, the solution obtained following ultrafiltration (UF) as disclosed in the present section above is further treated by diafiltration (UF/DF treatment).

Diafiltration (DF) is used to exchange product into a desired buffer solution (or water only). In an embodiment, diafiltration is used to change the chemical properties of the retained solution under constant volume. Unwanted particles pass through a membrane while the make-up of the feed stream is changed to a more desirable state through the addition of a replacement solution (a buffer solution, a saline solution, a buffer saline solution or water).

In an embodiment, the replacement solution is water.

In an embodiment, the replacement solution is saline in water. In some embodiments, the salt is selected from the group consisting of magnesium chloride, potassium chloride, sodium chloride and a combination thereof. In one particular embodiment, the salt is sodium chloride. In one embodiment, the replacement solution is sodium chloride at about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM or about 500 mM. In one particular embodiment, the replacement solution is sodium chloride at about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM or about 300 mM.

In an embodiment, the replacement solution is a buffer solution. In an embodiment, the replacement solution is a buffer solution wherein the buffer is selected from the group consisting of N-(2-Acetamido)-aminoethanesulfonic acid (ACES), a salt of acetic acid (acetate), N-(2-Acetamido)-iminodiacetic acid (ADA), 2-Aminoethanesulfonic acid (AES, Taurine), ammonia, 2-Amino-2-methyl-1-propanol (AMP), 2-Amino-2-methyl-1,3-propanediol AMPD, ammediol, N-(1,1-Dimethyl-2-hydroxyethyl)-3-amino-2-hydroxypropanesulfonic acid (AMPSO), N,N-Bis-(2-hydroxyethyl)-2-aminoethanesulfonic acid (BES), sodium hydrogen carbonate (bicarbonate), N,N′-Bis(2-hydroxyethyl)-glycine (bicine), [Bis-(2-hydroxyethyl)-imino]-tris-(hydroxymethyl methane) (BIS-Tris), 1,3-Bis[tris(hydroxymethyl)-methylamino]propane (BIS-Tris-Propane), Boric acid, dimethylarsinic acid (Cacodylate), 3-(Cyclohexylamino)-propanesulfonic acid (CAPS), 3-(Cyclohexylamino)-2-hydroxy-1-propanesulfonic acid (CAPSO), sodium carbonate (Carbonate), cyclohexylaminoethanesulfonic acid (CHES), a salt of citric acid (citrate), 3-[N-Bis(hydroxyethyl)amino]-2-hydroxypropanesulfonic acid (DIPSO), a salt of formic acid (formate), Glycine, Glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, a salt of malic acid (Malate), a salt of maleic acid (Maleate), 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), a salt of phosphoric acid (Phosphate), Piperazine-N,N′-bis(2-ethanesulfonic acid) (PIPES), Piperazine-N,N′-bis(2-hydroxypropanesulfonic acid) (POPSO), pyridine, a salt of succinic acid (Succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine) and Tris(hydroxymethyl)-aminomethane (Tris).

In an embodiment, the diafiltration buffer is selected from the group consisting of a salt of acetic acid (acetate), a salt of citric acid (citrate), a salt of formic acid (formate), a salt of malic acid (Malate), a salt of maleic acid (Maleate), a salt of phosphoric acid (Phosphate) and a salt of succinic acid (Succinate). In an embodiment, the diafiltration buffer is a salt of citric acid (citrate). In an embodiment, the diafiltration buffer is a salt of succinic acid (Succinate). In an embodiment, said salt is a sodium salt. In an embodiment, said salt is a potassium salt.

In an embodiment, the pH of the diafiltration buffer is between about 4.0-11.0, between about 5.0-10.0, between about 5.5-9.0, between about 6.0-8.0, between about 6.0-7.0, between about 6.5-7.5, between about 6.5-7.0 or between about 6.0-7.5. Any number within any of the above ranges is contemplated as an embodiment of the disclosure. In an embodiment, the pH of the diafiltration buffer is about 4.0, about 4.5, about 5.0, about 5.5, about 6.0, about 6.5, about 7.0, about 7.5, about 8.0, about 8.5, about 9.0, about 9.5, about 10.0, about 10.5 or about 11.0. In an embodiment, the pH of the diafiltration buffer is about 6.0, about 6.5, about 7.0, about 7.5, about 8.0, about 8.5 or about 9.0. In an embodiment, the pH of the diafiltration buffer is about 6.5, about 7.0 or about 7.5. In an embodiment, the pH of the diafiltration buffer is about 7.0.

In an embodiment, the concentration of the diafiltration buffer is between about 0.01 mM-100 mM, between about 0.1 mM-100 mM, between about 0.5 mM-100 mM, between about 1 mM-100 mM, between about 2 mM-100 mM, between about 3 mM-100 mM, between about 4 mM-100 mM, between about 5 mM-100 mM, between about 6 mM-100 mM, between about 7 mM-100 mM, between about 8 mM-100 mM, between about 9 mM-100 mM, between about 10 mM-100 mM, between about 11 mM-100 mM, between about 12 mM-100 mM, between about 13 mM-100 mM, between about 14 mM-100 mM, between about 15 mM-100 mM, between about 16 mM-100 mM, between about 17 mM-100 mM, between about 18 mM-100 mM, between about 19 mM-100 mM, between about 20 mM-100 mM, between about 25 mM-100 mM, between about 30 mM-100 mM, between about 35 mM-100 mM, between about 40 mM-100 mM, between about 45 mM-100 mM, between about 50 mM-100 mM, between about 55 mM-100 mM, between about 60 mM-100 mM, between about 65 mM-100 mM, between about 70 mM-100 mM, between about 75 mM-100 mM, between about 80 mM-100 mM, between about 85 mM-100 mM, between about 90 mM-100 mM or between about 95 mM-100 mM.

In an embodiment, the concentration of the diafiltration buffer is between about 0.01 mM-50 mM, between about 0.1 mM-50 mM, between about 0.5 mM-50 mM, between about 1 mM-50 mM, between about 2 mM-50 mM, between about 3 mM-50 mM, between about 4 mM-50 mM, between about 5 mM-50 mM, between about 6 mM-50 mM, between about 7 mM-50 mM, between about 8 mM-50 mM, between about 9 mM-50 mM, between about 10 mM-50 mM, between about 11 mM-50 mM, between about 12 mM-50 mM, between about 13 mM-50 mM, between about 14 mM-50 mM, between about 15 mM-50 mM, between about 16 mM-50 mM, between about 17 mM-50 mM, between about 18 mM-50 mM, between about 19 mM-50 mM, between about 20 mM-50 mM, between about 25 mM-50 mM, between about 30 mM-50 mM, between about 35 mM-50 mM, between about 40 mM-50 mM or between about 45 mM-50 mM.

In an embodiment, the concentration of the diafiltration buffer is between about 0.01 mM-25 mM, between about 0.1 mM-25 mM, between about 0.5 mM-25 mM, between about 1 mM-25 mM, between about 2 mM-25 mM, between about 3 mM-25 mM, between about 4 mM-25 mM, between about 5 mM-25 mM, between about 6 mM-25 mM, between about 7 mM-25 mM, between about 8 mM-25 mM, between about 9 mM-25 mM, between about 10 mM-25 mM, between about 11 mM-25 mM, between about 12 mM-25 mM, between about 13 mM-25 mM, between about 14 mM-25 mM, between about 15 mM-25 mM, between about 16 mM-25 mM, between about 17 mM-25 mM, between about 18 mM-25 mM, between about 19 mM-25 mM or between about 20 mM-25 mM.

In an embodiment, the concentration of the diafiltration buffer is between about 0.01 mM-15 mM, between about 0.1 mM-15 mM, between about 0.5 mM-15 mM, between about 1 mM-15 mM, between about 2 mM-15 mM, between about 3 mM-15 mM, between about 4 mM-15 mM, between about 5 mM-15 mM, between about 6 mM-15 mM, between about 7 mM-15 mM, between about 8 mM-15 mM, between about 9 mM-15 mM, between about 10 mM-15 mM, between about 11 mM-15 mM, between about 12 mM-15 mM, between about 13 mM-15 mM or between about 14 mM-15 mM.

In an embodiment, the concentration of the diafiltration buffer is between about 0.01 mM-10 mM, between about 0.1 mM-10 mM, between about 0.5 mM-10 mM, between about 1 mM-10 mM, between about 2 mM-10 mM, between about 3 mM-10 mM, between about 4 mM-10 mM, between about 5 mM-10 mM, between about 6 mM-10 mM, between about 7 mM-10 mM, between about 8 mM-10 mM or between about 9 mM-10 mM.

Any number within any of the above ranges is contemplated as an embodiment of the disclosure.

In an embodiment, the concentration of the diafiltration buffer is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.

In an embodiment, the concentration of the diafiltration buffer is about 0.1 mM, about 0.2 mM, about 1 mM, about 5 mM, about 10 mM, about 15 mM, about 20 mM, about 30 mM, about 40 mM, or about 50 mM.

In an embodiment, the concentration of the diafiltration buffer is about 10 mM.

In an embodiment, the replacement solution comprises a chelating agent. In an embodiment, the replacement solution comprises an alum chelating agent. In some embodiments, the chelating agent is selected from the groups consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N,N, N′,N′-tetraacetic acid (DPTA-OH), ethylenediamine-N,N′-bis(2-hydroxyphenylacetic acid) (EDDHA), ethylenediamine-N,N′-dipropionic acid dihydrochloride (EDDP), ethylenediamine-tetrakis(methylenesulfonic acid) (EDTPO), Nitrilotris(methylenephosphonic acid) (NTPO), imino-diacetic acid (IDA), hydroxyimino-diacetic acid (HIDA), nitrilo-triacetic acid (NTP), triethylenetetramine-hexaacetic acid (TTHA), Dimercaptosuccinic acid (DMSA), 2,3-dimercapto-1-propanesulfonic acid (DMPS), alpha lipoic acid (ALA), Nitrilotriacetic acid (NTA), thiamine tetrahydrofurfuryl disulfide (TTFD), dimercaprol, penicillamine, deferoxamine (DFOA), deferasirox, phosphonates, a salt of citric acid (citrate) and combinations of these.

In some embodiments, the chelating agent is selected from the groups consisting of

Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N, N,N′,N″,N″-pentaacetic acid (DTPA), 1,3-diaminopropan-2-ol-N,N,N′,N′-tetraacetic acid (DPTA-OH), ethylenediamine-N,N′-bis(2-hydroxyphenylacetic acid) (EDDHA), a salt of citric acid (citrate) and combinations of these.

In some embodiments, the chelating agent is Ethylene Diamine Tetra Acetate (EDTA). In some embodiments, the chelating agent is a salt of citric acid (citrate). In some embodiments, the chelating agent is sodium citrate.

In general, the chelating agent is employed at a concentration from 1 to 500 mM. In an embodiment, the concentration of the chelating agent in the replacement solution is from 2 to 400 mM. In an embodiment, the concentration of the chelating agent in the replacement solution is from 10 to 400 mM. In an embodiment, the concentration of the chelating agent in the replacement solution is from 10 to 200 mM.

In an embodiment, the concentration of the chelating agent in the replacement solution is from 10 to 100 mM. In an embodiment, the concentration of the chelating agent in the replacement solution is from 10 to 50 mM. In an embodiment, the concentration of the chelating agent in the replacement solution is from 10 to 30 mM.

In an embodiment, the concentration of the chelating agent in the replacement solution is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.

In an embodiment, the concentration of the chelating agent in the replacement solution is about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM or about 100 mM.

In an embodiment, the concentration of the chelating agent in the replacement solution is about 15 mM, about 20 mM, about 25 mM, about 30 mM, about 35 mM, about 40 mM, about 45 mM or about 50 mM.

In an embodiment, the diafiltration buffer solution comprises a salt. In some embodiments, the salt is selected from the groups consisting of magnesium chloride, potassium chloride, sodium chloride and a combination thereof. In one particular embodiment, the salt is sodium chloride. In an embodiment, the diafiltration buffer solution comprises sodium chloride at about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250, about 300, about 350, about 400, about 450 or about 500 mM. In one particular embodiment, the diafiltration buffer solution comprises sodium chloride at about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250 or about 300 mM.

In an embodiment of the present invention, the number of diavolumes is at least 5, 10, 15, 20, 25, 30, 35, 40, 45, or 50. In an embodiment of the present invention, the number of diavolumes is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95 or about 100. In an embodiment of the present invention the number of diavolumes is about 5, about 6, about 7, about 8, about 9, about 10, about 11, about 12, about 13, about 14 or about 15.

In an embodiment of the present invention, the Ultrafiltration and Dialfiltration steps are performed at a temperature between about 20° C. to about 90° C. In an embodiment, the Ultrafiltration and Dialfiltration steps are performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.

Any number within any of the above ranges is contemplated as an embodiment of the disclosure.

In an embodiment, the Ultrafiltration and Dialfiltration steps are performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Ultrafiltration and Dialfiltration step are performed at a temperature of about 50° C.

In an embodiment of the present invention, the Dialfiltration step is performed at temperature between about 20° C. to about 90° C. In an embodiment, the Dialfiltration step is performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.

Any number within any of the above ranges is contemplated as an embodiment of the disclosure.

In an embodiment, Dialfiltration step is performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Dialfiltration step is performed at a temperature of about 50° C.

In an embodiment of the present invention, the Ultrafiltration step is performed at temperature between about 20° C. to about 90° C. In an embodiment, the Ultrafiltration step is performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C. Any number within any of the above ranges is contemplated as an embodiment of the disclosure.

In an embodiment, Ultrafiltration step is performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Ultrafiltration step is performed at a temperature of about 50° C.

1.7 Activated Carbon Filtration

Once the solution has been treated by the flocculation step of section 1.2 above, the solution containing the polysacharide can optionally be further clarified by an activated carbon filtration step.

In an embodiment, the solution of section 1.3 further treated by the solid/liquid separation of step of section 1.3 (e.g. the supernatant) is further clarified by an activated carbon filtration step. In an embodiment, the solution further filtered by any of the method of section 1.4 above and/or by the filtration step of section 1.5 above is further clarified by an activated carbon filtration step. In an embodiment, the solution further clarified by an Ultrafiltration and/or Dialfiltration step of section 1.6 above is further clarified by an activated carbon filtration step.

A step of activated carbon filtration allows for further removing host cell impurities such as proteins and nucleic acids as well as colored impurities (see WO2008/118752).

In an embodiment, activated carbon (also named active charcoal) is added to the solution in an amount sufficient to absorb the majority of the proteins and nucleic acids contaminants, and then removed once the contaminants have been adsorbed onto activated carbon. In an embodiment the activated carbon is added in the form of a powder, as a granular carbon bed, as a pressed carbon block or extruded carbon block (see e.g. Norit active charcoal). In an embodiment, the activated carbon is added in an amount of about 0.1 to 20% (weight volume), 1 to 15% (weight volume), 1 to 10% (weight volume), 2 to 10% (weight volume), 3 to 10% (weight volume), 4 to 10% (weight volume), 5 to 10% (weight volume), 1 to 5% (weight volume) or 2 to 5% (weight volume). The mixture is then stirred and left to stand. In an embodiment, the mixture is left to stand for about 5, 10, 15, 20, 30, 45, 60, 90, 120, 180, 240 minutes or more. The activated carbon is then removed. The activated carbon can be removed for example by centrifugation or filtration.

In a preferred embodiment, the solution is filtered through activated carbon immobilized in a matrix. The matrix may be any porous filter medium permeable for the solution. The matrix may comprise a support material and/or a binder material. The support material may be a synthetic polymer or a polymer of natural origin. Suitable synthetic polymers may include polystyrene, polyacrylamide and polymethyl methacrylate, while polymers of natural origin may include cellulose, polysaccharide and dextran, agarose. Typically, the polymer support material is in the form of a fibre network to provide mechanical rigidity. The binder material may be a resin. The matrix may have the form of a membrane sheet. In an embodiment, the activated carbon immobilized in the matrix is in the form of a flow-through carbon cartridge. A cartridge is a self-contained entity containing powdered activated carbon immobilized in the matrix and prepared in the form of a membrane sheet. The membrane sheet may be captured in a plastic permeable support to form a disc.

Alternatively, the membrane sheet may be spirally wound. To increase filter surface area, several discs may be stacked upon each other. In particular, the discs stacked upon each other have a central core pipe for collecting and removing the carbon-treated sample from the filter. The configuration of stacked discs may be lenticular. The activated carbon in the carbon filter may be derived from different raw materials, e.g. peat, lignite, wood or coconut shell.

Any process known in the art, such as steam or chemical treatment, may be used to activate carbon (e.g. wood-based phosphoric acid-activated carbon).

In the present invention, activated carbon immobilized in a matrix may be placed in a housing to form an independent filter unit. Each filter unit has its own in-let and out-let for the solution to be purified. Examples of filter units that are usable in the present invention are the carbon cartridges from Cuno Inc. (Meriden, USA) or Pall Corporation (East Hill, USA). In particular, CUNO zetacarbon filters are suitable for use in the invention. These carbon filters comprise a cellulose matrix into which activated carbon powder is entrapped and resin-bonded in place.

In an embodiment, the activated carbon filter disclosed above has a nominal micron rating of between about 0.01-100 micron, about 0.05-100 micron, about 0.1-100 micron, about 0.2-100 micron, about 0.3-100 micron, about 0.4-100 micron, about 0.5-100 micron, about 0.6-100 micron, about 0.7-100 micron, about 0.8-100 micron, about 0.9-100 micron, about 1-100 micron, about 1.25-100 micron, about 1.5-100 micron, about 1.75-100 micron, about 2-100 micron, about 3-100 micron, about 4-100 micron, about 5-100 micron, about 6-100 micron, about 7-100 micron, about 8-100 micron, about 9-100 micron, about 10-100 micron, about 15-100 micron, about 20-100 micron, about 25-100 micron, about 30-100 micron, about 40-100 micron, about 50-100 micron or about 75-100 micron.

In an embodiment, the activated carbon filter disclosed above has a nominal micron rating of between about 0.01-50 micron, about 0.05-50 micron, about 0.1-50 micron, about 0.2-50 micron, about 0.3-50 micron, about 0.4-50 micron, about 0.5-50 micron, about 0.6-50 micron, about 0.7-50 micron, about 0.8-50 micron, about 0.9-50 micron, about 1-50 micron, about 1.25-50 micron, about 1.5-50 micron, about 1.75-50 micron, about 2-50 micron, about 3-50 micron, about 4-50 micron, about 5-50 micron, about 6-50 micron, about 7-50 micron, about 8-50 micron, about 9-50 micron, about 10-50 micron, about 15-50 micron, about 20-50 micron, about 25-50 micron, about 30-50 micron, about 40-50 micron or about 50-50 micron.

In an embodiment, the activated carbon filter disclosed above has a nominal micron rating of between about 0.01-25 micron, about 0.05-25 micron, about 0.1-25 micron, about 0.2-25 micron, about 0.3-25 micron, about 0.4-25 micron, about 0.5-25 micron, about 0.6-25 micron, about 0.7-25 micron, about 0.8-25 micron, about 0.9-25 micron, about 1-25 micron, about 1.25-25 micron, about 1.5-25 micron, about 1.75-25 micron, about 2-25 micron, about 3-25 micron, about 4-25 micron, about 5-25 micron, about 6-25 micron, about 7-25 micron, about 8-25 micron, about 9-25 micron, about 10-25 micron, about 15-25 micron or about 20-25 micron.

In an embodiment, the activated carbon filter disclosed above has a nominal micron rating of between about 0.01-10 micron, about 0.05-10 micron, about 0.1-10 micron, about 0.2-10 micron, about 0.3-10 micron, about 0.4-10 micron, about 0.5-10 micron, about 0.6-10 micron, about 0.7-10 micron, about 0.8-10 micron, about 0.9-10 micron, about 1-10 micron, about 1.25-10 micron, about 1.5-10 micron, about 1.75-10 micron, about 2-10 micron, about 3-10 micron, about 4-10 micron, about 5-10 micron, about 6-10 micron, about 7-10 micron, about 8-10 micron or about 9-10 micron.

In an embodiment, the activated carbon filter disclosed above has a nominal micron rating of between about 0.01-8 micron, about 0.05-8 micron, about 0.1-8 micron, about 0.2-8 micron, about 0.3-8 micron, about 0.4-8 micron, about 0.5-8 micron, about 0.6-8 micron, about 0.7-8 micron, about 0.8-8 micron, about 0.9-8 micron, about 1-8 micron, about 1.25-8 micron, about 1.5-8 micron, about 1.75-8 micron, about 2-8 micron, about 3-8 micron, about 4-8 micron, about 5-8 micron, about 6-8 micron or about 7-8 micron.

In an embodiment, the activated carbon filter disclosed above has a nominal micron rating of between about 0.01-5 micron, about 0.05-5 micron, about 0.1-5 micron, about 0.2-5 micron, about 0.3-5 micron, about 0.4-5 micron, about 0.5-5 micron, about 0.6-5 micron, about 0.7-5 micron, about 0.8-5 micron, about 0.9-5 micron, about 1-5 micron, about 1.25-5 micron, about 1.5-5 micron, about 1.75-5 micron, about 2-5 micron, about 3-5 micron or about 4-5 micron.

In an embodiment, the activated carbon filter disclosed above has a nominal micron rating of between about 0.01-2 micron, about 0.05-2 micron, about 0.1-2 micron, about 0.2-2 micron, about 0.3-2 micron, about 0.4-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, about 1.75-2 micron, about 2-2 micron, about 3-2 micron or about 4-2 micron.

In an embodiment, the activated carbon filter disclosed above has a nominal micron rating of between about 0.01-1 micron, about 0.05-1 micron, about 0.1-1 micron, about 0.2-1 micron, about 0.3-1 micron, about 0.4-1 micron, about 0.5-1 micron, about 0.6-1 micron, about 0.7-1 micron, about 0.8-1 micron or about 0.9-1 micron.

In an embodiment, the activated carbon filter disclosed above has a nominal micron ratings of between about 0.05-50 micron, 0.1-25 micron 0.2-10, micron 0.1-10 micron, 0.2-5 micron or 0.25-1 micron.

Any number within any of the above ranges is contemplated as an embodiment of the disclosure.

In an embodiment, the activated carbon filtration step is conducted at a feed rate of between 1-500 LMH, 10-500 LMH, 15-500 LMH, 20-500 LMH, 25-500 LMH, 30-500 LMH, 40-500 LMH, 50-500 LMH, 100-500 LMH, 125-500 LMH, 150-500 LMH, 200-500

LMH, 250-500 LMH, 300-500 LMH or 400-500 LMH.

In an embodiment, the activated carbon filtration step is conducted at a feed rate of between 1-200 LMH, 10-200 LMH, 15-200 LMH, 20-200 LMH, 25-200 LMH, 30-200 LMH, 40-200 LMH, 50-200 LMH, 100-200 LMH, 125-200 LMH or 150-200 LMH.

In an embodiment, the activated carbon filtration step is conducted at a feed rate of between 1-150 LMH, 10-150 LMH, 15-150 LMH, 20-150 LMH, 25-150 LMH, 30-150 LMH, 40-150 LMH, 50-150 LMH, 100-150 LMH or 125-150 LMH.

In an embodiment, the activated carbon filtration step is conducted at a feed rate of between 1-100 LMH, 10-100 LMH, 15-100 LMH, 20-100 LMH, 25-100 LMH, 30-100 LMH, 40-100 LMH, or 50-100 LMH.

In an embodiment, the activated carbon filtration step is conducted at a feed rate of between 1-75 LMH, 5-75 LMH, 10-75 LMH, 15-75 LMH, 20-75 LMH, 25-75 LMH, 30-75 LMH, 35-75 LMH, 40-75 LMH, 45-75 LMH, 50-75 LMH, 55-75 LMH, 60-75 LMH, 65-75 LMH, or 70-75 LMH.

In an embodiment, the activated carbon filtration step is conducted at a feed rate of between 1-50 LMH, 5-50 LMH, 7-50 LMH, 10-50 LMH, 15-50 LMH, 20-50 LMH, 25-50 LMH, 30-50 LMH, 35-50 LMH, 40-50 LMH or 45-50 LMH.

Any whole number integer within any of the above ranges is contemplated as an embodiment of the disclosure.

In an embodiment, the activated carbon filtration step is conducted at a feed rate of about 1, about 2, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 300, about 350, about 400, about 450, about 500, about 550, about 600, about 700, about 800, about 900, about 950 or about 1000 LMH.

In an embodiment, the solution is treated by an activated carbon filter wherein the filter has a filter capacity of between 5-1000 L/m², 10-750 L/m², 15-500 L/m², 20-400 L/m², 25-300 L/m², 30-250 L/m², 40-200 L/m² or 30-100 L/m².

Any number within any of the above ranges is contemplated as an embodiment of the disclosure.

In an embodiment, the solution is treated by an activated carbon filter wherein the filter has a filter capacity of about 5, about 10, about 15, about 20, about 25, about 30, about 35, about 40, about 45, about 50, about 55, about 60, about 65, about 70, about 75, about 80, about 85, about 90, about 100, about 125, about 150, about 175, about 200, about 225, about 250, about 275, about 300, about 400, about 500, about 600, about 700, about 800, about 900, or about 1000 L/m².

If the content of contaminants is above the fixed threshold after a first activated carbon filtration step, the said step can be repeated. In an embodiment of the present invention, 1, 2, 3, 4, 5, 6, 7, 8, 9, or 10 activated carbon filtration step(s) are performed. In an embodiment of the present invention, 1, 2 or 3 activated carbon filtration step(s) are performed. In an embodiment of the present invention, 1 or 2 activated carbon filtration step(s) are performed.

In an embodiment, the solution is treated by activated carbon filters in series. In an embodiment, the solution is treated by 1, 2, 3, 4, 5, 6, 7, 8, 9, or 10 activated carbon filters in series. In an embodiment, the solution is treated by 2, 3, 4 or 5 activated carbon filters in series. In an embodiment, the solution is treated by 2 activated carbon filters in series. In an embodiment, the solution is treated by 3 activated carbon filters in series. In an embodiment, the solution is treated by 4 activated carbon filters in series. In an embodiment, the solution is treated by 5 activated carbon filters in series.

In an embodiment the activated carbon filtration step is performed in a single pass mode.

In another embodiment the activated carbon filtration step is performed in recirculation mode. In said embodiment (recirculation mode) 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 cycles of activated carbon filtration are performed. In another embodiment 2, 3, 4, 5, 6, 7, 8, 9 or 10 cycles of activated carbon filtration are performed. In an embodiment, 2 or 3 cycles of activated carbon filtration are performed. In an embodiment, 2 cycles of activated carbon filtration are performed.

1.8 Optional Further Filtration

Once the solution has been treated by the activated carbon step of section 1.7 above, the obtained solution (i.e. the filtrate) can optionally be further filtered.

In an embodiment, the solution is subjected to microfiltration. In an embodiment, microfiltration is dead-end filtration (perpendicular filtration).

In an embodiment, the solution is treated by a microfiltration step wherein the filter has a nominal retention range of between about 0.01-2 micron, about 0.05-2 micron, about 0.1-2 micron, about 0.2-2 micron, about 0.3-2 micron, about 0.4-2 micron, about 0.45-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, or about 1.75-2 micron.

In an embodiment, the solution is treated by a microfiltration step wherein the filter has a nominal retention range of between about 0.01-1 micron, about 0.05-1 micron, about 0.1-1 micron, about 0.2-1 micron, about 0.3-1 micron, about 0.4-1 micron, about 0.45-1 micron, about 0.5-1 micron, about 0.6-1 micron, about 0.7-1 micron, about 0.8-1 micron or about 0.9-1 micron.

Any number within any of the above ranges is contemplated as an embodiment of the disclosure.

In an embodiment, the solution is treated by a microfiltration step wherein the filter has a nominal retention rating of about 0.01, about 0.05, about 0.1, about 0.2, about 0.3, about 0.4, about 0.45, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9 or about 2.0 micron.

In an embodiment, the solution is treated by a microfiltration step wherein the filter has a nominal retention rating of about 0.2 micron.

In an embodiment, the solution is treated by a microfiltration step wherein the filter has a filter capacity of 100-6000 L/m², 200-6000 L/m², 300-6000 L/m², 400-6000 L/m², 500-6000 L/m², 750-6000 L/m², 1000-6000 L/m², 1500-6000 L/m², 2000-6000 L/m², 3000-6000 L/m² or 4000-6000 L/m².

In an embodiment, the solution is treated by a microfiltration step wherein the filter has a filter capacity of 100-4000 L/m², 200-4000 L/m², 300-4000 L/m², 400-4000 L/m², 500-4000 L/m², 750-4000 L/m², 1000-4000 L/m², 1500-4000 L/m², 2000-4000 L/m², 2500-4000 L/m², 3000-4000 L/m², 3000-4000 L/m² or 3500-4000 L/m².

In an embodiment, the solution is treated by a microfiltration step wherein the filter has a filter capacity of 100-3750 L/m², 200-3750 L/m², 300-3750 L/m², 400-3750 L/m², 500-3750 L/m², 750-3750 L/m², 1000-3750 L/m², 1500-3750 L/m², 2000-3750 L/m², 2500-3750 L/m², 3000-3750 L/m², 3000-3750 L/m² or 3500-3750 L/m².

In an embodiment, the solution is treated by a microfiltration step wherein the filter has a filter capacity of 100-1250 L/m², 200-1250 L/m², 300-1250 L/m², 400-1250 L/m², 500-1250 L/m², 750-1250 L/m² or 1000-1250 L/m².

Any number within any of the above ranges is contemplated as an embodiment of the disclosure.

In an embodiment, the solution is treated by a microfiltration step wherein the filter has a filter capacity of about 100, about 200, about 300, about 400, about 550, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5250, about 5500, about 5750 or about 6000 L/m².

1.9 Ultrafiltration/Diafiltration

Once the solution has been treated by the activated carbon filtration step of section 1.7 above and/or by the further filtration step of section 1.8 above, the obtained solution (i.e. the filtrate) can optionally be further clarified by Ultrafiltration and/or Dialfiltration.

In an embodiment of the present invention, the solution (e.g. obtained at section 1.7 or 1.8 above) is treated by ultrafiltration.

In an embodiment, the solution is treated by ultrafiltration and the molecular weight cut off of the membrane is in the range of between about 5 kDa-1000 kDa. In an embodiment the molecular weight cut off of the membrane is in the range of between about 10 kDa-750 kDa. In an embodiment the molecular weight cut off of the membrane is in the range of between about 10 kDa-500 kDa. In an embodiment the molecular weight cut off of the membrane is in the range of between about 10 kDa-300 kDa. In an embodiment the molecular weight cut off of the membrane is in the range of between about 10 kDa-100 kDa. In an embodiment the molecular weight cut off of the membrane is in the range of between about 10 kDa-50 kDa. In an embodiment the molecular weight cut off of the membrane is in the range of between about 10 kDa-30 kDa. In an embodiment the molecular weight cut off of the membrane is in the range of between about 5 kDa-1000 kDa, about 10 kDa-1000 kDa about 20 kDa-1000 kDa, about 30 kDa-1000 kDa, about 40 kDa-1000 kDa, about 50 kDa-1000 kDa, about 75 kDa-1000 kDa, about 100 kDa-1000 kDa, about 150 kDa-1000 kDa, about 200 kDa-1000 kDa, about 300 kDa-1000 kDa, about 400 kDa-1000 kDa, about 500 kDa-1000 kDa or about 750 kDa-1000 kDa.

In an embodiment the molecular weight cut off of the membrane is in the range of between about 5 kDa-500 kDa, about 10 kDa-500 kDa, about 20 kDa-500 kDa, about 30 kDa-500 kDa, about 40 kDa-500 kDa, about 50 kDa-500 kDa, about 75 kDa-500 kDa, about 100 kDa-500 kDa, about 150 kDa-500 kDa, about 200 kDa-500 kDa, about 300 kDa-500 kDa or about 400 kDa-500 kDa.

In an embodiment the molecular weight cut off of the membrane is in the range of between about 5 kDa-300 kDa, about 10 kDa-300 kDa, about 20 kDa-300 kDa, about 30 kDa-300 kDa, about 40 kDa-300 kDa, about 50 kDa-300 kDa, about 75 kDa-300 kDa, about 100 kDa-300 kDa, about 150 kDa-300 kDa or about 200 kDa-300 kDa.

In an embodiment the molecular weight cut off of the membrane is in the range of between about 5 kDa-100 kDa, about 10 kDa-100 kDa, about 20 kDa-100 kDa, about 30 kDa-100 kDa, about 40 kDa-100 kDa, about 50 kDa-100 kDa or about 75 kDa-100 kDa.

In an embodiment the molecular weight cut off of the membrane is about 5 kDa, about 10 kDa, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 200 kDa, about 250 kDa, about 300 kDa, about 400 kDa, about 500 kDa, about 750 kDa or about 1000 kDa.

In an embodiment, the concentration factor of the ultrafiltration step is from about 1.5 to about 10.0. In an embodiment, the concentration factor is from about 2.0 to about 8.0. In an embodiment, the concentration factor is from about 2.0 to about 5.0.

In an embodiment, the concentration factor is about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0. In an embodiment, the concentration factor is about 2.0, about 3.0, about 4.0, about 5.0, or about 6.0.

In an embodiment of the present invention, the solution (e.g. the filtrate obtained at section 1.7 or 1.8 above) is treated by diafiltration.

In an embodiment of the present invention, the solution obtained following ultrafiltration (UF) as disclosed in the present section above is further treated by diafiltration (UF/DF treatment).

Diafiltration (DF) is used to exchange product into a desired buffer solution (or water only). In an embodiment, diafiltration is used to change the chemical properties of the retained solution under constant volume. Unwanted particles pass through a membrane while the make-up of the feed stream is changed to a more desirable state through the addition of a replacement solution (a buffer solution, a saline solution, a buffer saline solution or water).

In an embodiment, the replacement solution is water.

In an embodiment, the replacement solution is saline in water. In some embodiments, the salt is selected from the groups consisting of magnesium chloride, potassium chloride, sodium chloride and a combination thereof. In one particular embodiment, the salt is sodium chloride. In an embodiment, the replacement solution is sodium chloride at about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250, about 300, about 350, about 400, about 450 or about 500 mM. In one particular embodiment, the replacement solution is sodium chloride at about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250 or about 300 mM. In one particular embodiment, the replacement solution is sodium chloride at about 25, about 30, about 35, about 40, about 45, about 50, about 55, about 60, about 65, about 70, about 80, about 90 or about 100 mM.

In an embodiment, the replacement solution is a buffer solution. In an embodiment, the replacement solution is a buffer solution wherein the buffer is selected from the group consisting of N-(2-Acetamido)-aminoethanesulfonic acid (ACES), a salt of acetic acid (acetate), N-(2-Acetamido)-iminodiacetic acid (ADA), 2-Aminoethanesulfonic acid (AES, Taurine), ammonia, 2-Amino-2-methyl-1-propanol (AMP), 2-Amino-2-methyl-1,3-propanediol AMPD, ammediol, N-(1,1-Dimethyl-2-hydroxyethyl)-3-amino-2-hydroxypropanesulfonic acid (AMPSO), N,N-Bis-(2-hydroxyethyl)-2-aminoethanesulfonic acid (BES), sodium hydrogen carbonate (bicarbonate), N,N′-Bis(2-hydroxyethyl)-glycine (bicine), [Bis-(2-hydroxyethyl)-imino]-tris-(hydroxymethyl methane) (BIS-Tris), 1,3-Bis[tris(hydroxymethyl)-methylamino]propane (BIS-Tris-Propane), Boric acid, dimethylarsinic acid (Cacodylate), 3-(Cyclohexylamino)-propanesulfonic acid (CAPS), 3-(Cyclohexylamino)-2-hydroxy-1-propanesulfonic acid (CAPSO), sodium carbonate (Carbonate), cyclohexylaminoethanesulfonic acid (CHES), a salt of citric acid (citrate), 3-[N-Bis(hydroxyethyl)amino]-2-hydroxypropanesulfonic acid (DIPSO), a salt of formic acid (formate), Glycine, Glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, a salt of malic acid (Malate), a salt of maleic acid (Maleate), 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), a salt of phosphoric acid (Phosphate), Piperazine-N,N′-bis(2-ethanesulfonic acid) (PIPES), Piperazine-N,N′-bis(2-hydroxypropanesulfonic acid) (POPSO), pyridine, a salt of succinic acid (Succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine) and Tris(hydroxymethyl)-aminomethane (Tris).

In an embodiment, the diafiltration buffer is selected from the group consisting of a salt of acetic acid (acetate), a salt of citric acid (citrate), a salt of formic acid (formate), a salt of malic acid (malate), a salt of maleic acid (maleate), a salt of phosphoric acid (phosphate) and a salt of succinic acid (succinate). In an embodiment, the diafiltration buffer is a salt of citric acid (citrate). In an embodiment, the diafiltration buffer is a salt of succinic acid (succinate). In an embodiment, the diafiltration buffer is a salt of phosphoric acid (phosphate). In an embodiment, said salt is a sodium salt. In an embodiment, said salt is a potassium salt.

In an embodiment, the pH of the diafiltration buffer is between about 4.0-11.0, between about 5.0-10.0, between about 5.5-9.0, between about 6.0-8.0, between about 6.0-7.0, between about 6.5-7.5, between about 6.5-7.0 or between about 6.0-7.5. Any number within any of the above ranges is contemplated as an embodiment of the disclosure. In an embodiment, the pH of the diafiltration buffer is about 4.0, about 4.5, about 5.0, about 5.5, about 6.0, about 6.5, about 7.0, about 7.5, about 8.0, about 8.5, about 9.0, about 9.5, about 10.0, about 10.5 or about 11.0. In an embodiment, the pH of the diafiltration buffer is about 6.0, about 6.5, about 7.0, about 7.5, about 8.0, about 8.5 or about 9.0. In an embodiment, the pH of the diafiltration buffer is about 6.5, about 7.0 or about 7.5. In an embodiment, the pH of the diafiltration buffer is about 6.0. In an embodiment, the pH of the diafiltration buffer is about 6.5. In an embodiment, the pH of the diafiltration buffer is about 7.0.

In an embodiment, the concentration of the diafiltration buffer is between about 0.01 mM-100 mM, between about 0.1 mM-100 mM, between about 0.5 mM-100 mM, between about 1 mM-100 mM, between about 2 mM-100 mM, between about 3 mM-100 mM, between about 4 mM-100 mM, between about 5 mM-100 mM, between about 6 mM-100 mM, between about 7 mM-100 mM, between about 8 mM-100 mM, between about 9 mM-100 mM, between about 10 mM-100 mM, between about 11 mM-100 mM, between about 12 mM-100 mM, between about 13 mM-100 mM, between about 14 mM-100 mM, between about 15 mM-100 mM, between about 16 mM-100 mM, between about 17 mM-100 mM, between about 18 mM-100 mM, between about 19 mM-100 mM, between about 20 mM-100 mM, between about 25 mM-100 mM, between about 30 mM-100 mM, between about 35 mM-100 mM, between about 40 mM-100 mM, between about 45 mM-100 mM, between about 50 mM-100 mM, between about 55 mM-100 mM, between about 60 mM-100 mM, between about 65 mM-100 mM, between about 70 mM-100 mM, between about 75 mM-100 mM, between about 80 mM-100 mM, between about 85 mM-100 mM, between about 90 mM-100 mM or between about 95 mM-100 mM.

In an embodiment, the concentration of the diafiltration buffer is between about 0.01 mM-50 mM, between about 0.1 mM-50 mM, between about 0.5 mM-50 mM, between about 1 mM-50 mM, between about 2 mM-50 mM, between about 3 mM-50 mM, between about 4 mM-50 mM, between about 5 mM-50 mM, between about 6 mM-50 mM, between about 7 mM-50 mM, between about 8 mM-50 mM, between about 9 mM-50 mM, between about 10 mM-50 mM, between about 11 mM-50 mM, between about 12 mM-50 mM, between about 13 mM-50 mM, between about 14 mM-50 mM, between about 15 mM-50 mM, between about 16 mM-50 mM, between about 17 mM-50 mM, between about 18 mM-50 mM, between about 19 mM-50 mM, between about 20 mM-50 mM, between about 25 mM-50 mM, between about 30 mM-50 mM, between about 35 mM-50 mM, between about 40 mM-50 mM or between about 45 mM-50 mM.

In an embodiment, the concentration of the diafiltration buffer is between about 0.01 mM-25 mM, between about 0.1 mM-25 mM, between about 0.5 mM-25 mM, between about 1 mM-25 mM, between about 2 mM-25 mM, between about 3 mM-25 mM, between about 4 mM-25 mM, between about 5 mM-25 mM, between about 6 mM-25 mM, between about 7 mM-25 mM, between about 8 mM-25 mM, between about 9 mM-25 mM, between about 10 mM-25 mM, between about 11 mM-25 mM, between about 12 mM-25 mM, between about 13 mM-25 mM, between about 14 mM-25 mM, between about 15 mM-25 mM, between about 16 mM-25 mM, between about 17 mM-25 mM, between about 18 mM-25 mM, between about 19 mM-25 mM or between about 20 mM-25 mM.

In an embodiment, the concentration of the diafiltration buffer is between about 0.01 mM-15 mM, between about 0.1 mM-15 mM, between about 0.5 mM-15 mM, between about 1 mM-15 mM, between about 2 mM-15 mM, between about 3 mM-15 mM, between about 4 mM-15 mM, between about 5 mM-15 mM, between about 6 mM-15 mM, between about 7 mM-15 mM, between about 8 mM-15 mM, between about 9 mM-15 mM, between about 10 mM-15 mM, between about 11 mM-15 mM, between about 12 mM-15 mM, between about 13 mM-15 mM or between about 14 mM-15 mM.

In an embodiment, the concentration of the diafiltration buffer is between about 0.01 mM-10 mM, between about 0.1 mM-10 mM, between about 0.5 mM-10 mM, between about 1 mM-10 mM, between about 2 mM-10 mM, between about 3 mM-10 mM, between about 4 mM-10 mM, between about 5 mM-10 mM, between about 6 mM-10 mM, between about 7 mM-10 mM, between about 8 mM-10 mM or between about 9 mM-10 mM.

Any number within any of the above ranges is contemplated as an embodiment of the disclosure.

In an embodiment, the concentration of the diafiltration buffer is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.

In an embodiment, the concentration of the diafiltration buffer is about 0.1 mM, about 0.2 mM, about 1 mM, about 5 mM, about 10 mM, about 15 mM, about 20 mM, about 25 mM, about 30 mM, about 40 mM, or about 50 mM. In an embodiment, the concentration of the diafiltration buffer is about 30 mM. In an embodiment, the concentration of the diafiltration buffer is about 25 mM. In an embodiment, the concentration of the diafiltration buffer is about 20 mM. In an embodiment, the concentration of the diafiltration buffer is about 15 mM. In an embodiment, the concentration of the diafiltration buffer is about 10 mM.

In an embodiment, the diafiltration buffer solution comprises a salt. In some embodiments, the salt is selected from the groups consisting of magnesium chloride, potassium chloride, sodium chloride and a combination thereof. In one particular embodiment, the salt is sodium chloride. In one particular embodiment, the diafiltration buffer solution comprises sodium chloride at about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250 or about 300 mM.

In an embodiment of the present invention, the number of diavolumes is at least 5, 10, 15, 20, 25, 30, 35, 40, 45, or 50. In an embodiment of the present invention, the number of diavolumes is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95 or about 100. In an embodiment of the present invention the number of diavolumes is about 5, about 6, about 7, about 8, about 9, about 10, about 11, about 12, about 13, about 14 or about 15.

In an embodiment of the present invention, the Ultrafiltration and Dialfiltration steps are performed at temperature between about 20° C. to about 90° C. In an embodiment, the Ultrafiltration and Dialfiltration steps are performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C. Any number within any of the above ranges is contemplated as an embodiment of the disclosure. In an embodiment, the Ultrafiltration and Dialfiltration steps are performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Ultrafiltration and Dialfiltration step are performed at a temperature of about 50° C.

In an embodiment of the present invention, the Dialfiltration step is performed at temperature between about 20° C. to about 90° C. In an embodiment, the Dialfiltration step is performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C. Any number within any of the above ranges is contemplated as an embodiment of the disclosure.

In an embodiment, Dialfiltration step is performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Dialfiltration step is performed at a temperature of about 50° C.

In an embodiment of the present invention, the Ultrafiltration step is performed at temperature between about 20° C. to about 90° C. In an embodiment, the Ultrafiltration step is performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C. Any number within any of the above ranges is contemplated as an embodiment of the disclosure.

In an embodiment, Ultrafiltration step is performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Ultrafiltration step is performed at a temperature of about 50° C.

1.10 Homogenization/Sizing

A polysaccharide can become slightly reduced in size during the purification procedures.

In an embodiment, the purified solution of polysaccharide of the present invention (e.g. obtained by Ultrafiltration and/or Dialfiltration of section 1.9) is not sized.

In an embodiment, the polysaccharide can be homogenized by sizing techniques. Mechanical or chemical sizing maybe employed. Chemical hydrolysis maybe conducted using for example acetic acid. Mechanical sizing maybe conducted using High Pressure Homogenization Shearing.

Therefore in an embodiment, the purified solution of polysaccharide obtained by Ultrafiltration and/or Dialfiltration of section 1.9 is sized to a target molecular weight.

As used herein, the term “molecular weight” of polysaccharide refers to molecular weight calculated for example by size exclusion chromatography (SEC) combined with multiangle laser light scattering detector (MALLS).

In some embodiments, the purified polysaccharide is sized to a molecular weight of between about 5 kDa and about 4,000 kDa. In other such embodiments, the purified polysaccharide is sized to a molecular weight of between about 10 kDa and about 4,000 kDa. In other such embodiments, the purified polysaccharide is sized to a molecular weight of between about 50 kDa and about 4,000 kDa. In further such embodiments, the polysaccharide the purified polysaccharide is sized to a molecular weight of between about 50 kDa and about 3,500 kDa; between about 50 kDa and about 3,000 kDa; between about 50 kDa and about 2,500 kDa; between about 50 kDa and about 2,000 kDa; between about 50 kDa and about 1,750 kDa; about between about 50 kDa and about 1,500 kDa; between about 50 kDa and about 1,250 kDa; between about 50 kDa and about 1,000 kDa; between about 50 kDa and about 750 kDa; between about 50 kDa and about 500 kDa; between about 100 kDa and about 4,000 kDa; between about 100 kDa and about 3,500 kDa; about 100 kDa and about 3,000 kDa; about 100 kDa and about 2,500 kDa; about 100 kDa and about 2,250 kDa; between about 100 kDa and about 2,000 kDa; between about 100 kDa and about 1,750 kDa; between about 100 kDa and about 1,500 kDa; between about 100 kDa and about 1,250 kDa; between about 100 kDa and about 1,000 kDa; between about 100 kDa and about 750 kDa; between about 100 kDa and about 500 kDa; between about 200 kDa and about 4,000 kDa; between about 200 kDa and about 3,500 kDa; between about 200 kDa and about 3,000 kDa; between about 200 kDa and about 2,500 kDa; between about 200 kDa and about 2,250 kDa; between about 200 kDa and about 2,000 kDa; between about 200 kDa and about 1,750 kDa; between about 200 kDa and about 1,500 kDa; between about 200 kDa and about 1,250 kDa; between about 200 kDa and about 1,000 kDa; between about 200 kDa and about 750 kDa; or between about 200 kDa and about 500 kDa. In further such embodiments, the polysaccharide the purified polysaccharide is sized to a molecular weight of between about 250 kDa and about 3,500 kDa; between about 250 kDa and about 3,000 kDa; between about 250 kDa and about 2,500 kDa; between about 250 kDa and about 2,000 kDa; between about 250 kDa and about 1,750 kDa; about between about 250 kDa and about 1,500 kDa; between about 250 kDa and about 1,250 kDa; between about 250 kDa and about 1,000 kDa; between about 250 kDa and about 750 kDa; between about 250 kDa and about 500 kDa; between about 300 kDa and about 4,000 kDa; between about 300 kDa and about 3,500 kDa; about 300 kDa and about 3,000 kDa; about 300 kDa and about 2,500 kDa; about 300 kDa and about 2,250 kDa; between about 300 kDa and about 2,000 kDa; between about 300 kDa and about 1,750 kDa; between about 300 kDa and about 1,500 kDa; between about 300 kDa and about 1,250 kDa; between about 300 kDa and about 1,000 kDa; between about 300 kDa and about 750 kDa; between about 300 kDa and about 500 kDa; between about 500 kDa and about 4,000 kDa; between about 500 kDa and about 3,500 kDa; between about 500 kDa and about 3,000 kDa; between about 500 kDa and about 2,500 kDa; between about 500 kDa and about 2,250 kDa; between about 500 kDa and about 2,000 kDa; between about 500 kDa and about 1,750 kDa; between about 500 kDa and about 1,500 kDa; between about 500 kDa and about 1,250 kDa; between about 500 kDa and about 1,000 kDa; between about 500 kDa and about 750 kDa; or between about 500 kDa and about 600 kDa.

Any number within any of the above ranges is contemplated as an embodiment of the disclosure.

In some embodiments, the purified polysaccharide is sized to a molecular weight of about 5 kDa, about 10 kDa, about 15 kDa, about 20 kDa, about 25 kDa, about 30 kDa, about 35 kDa, about 40 kDa, about 45 kDa, about 50 kDa, about 75 kDa, about 90 kDa, about 100 kDa, about 150 kDa, about 200 kDa, about 250 kDa, about 300 kDa, about 350 kDa, about 400 kDa, about 450 kDa, about 500 kDa, about 550 kDa, about 600 kDa, about 650 kDa, about 700 kDa, about 750 kDa, about 800 kDa, about 850 kDa, about 900 kDa, about 950 kDa, about 1000 kDa, about 1250 kDa, about 1500 kDa, about 1750 kDa, about 2000 kDa, about 2250 kDa, about 2500 kDa, about 2750 kDa, about 3000 kDa, about 3250 kDa, about 3500 kDa, about 3750 kDa or about 4,000 kDa.

In a preferred embodiment the purified polysaccharides, are capsular polysaccharide from serotypes 1, 3, 4, 5, 6A, 6B, 7F, 8, 9V, 10A, 11A, 12F, 14, 15A, 15B, 18C, 19A, 19F, 22F, 23F or 33F of S. pneumoniae, wherein the capsular polysaccharide has a molecular weight falling within one of the ranges or having about the size as described here above.

1.11 Sterile Filtration

In an embodiment, the purified solution of polysaccharide of the invention is sterilely filtered.

Therefore in an embodiment, the Ultrafiltration and/or Dialfiltration step of section 1.9 can optionally be followed by a sterile filtration step.

In an embodiment, the homogenizing/sizing step of section 1.10 if conducted can optionally be followed by a sterile filtration step.

In an embodiment, any of the step of sections 1.2 to 1.8 can optionally be followed by a sterile filtration step.

In an embodiment, sterile filtration is dead-end filtration (perpendicular filtration). In an embodiment, sterile filtration is tangential filtration.

In an embodiment, the solution is treated by a sterile filtration step wherein the filter has a nominal retention range of between about 0.01-0.2 micron, about 0.05-0.2 micron, about 0.1-0.2 micron or about 0.15-0.2 micron.

Any number within any of the above ranges is contemplated as an embodiment of the disclosure.

In an embodiment, the solution is treated by a sterile filtration step wherein the filter has a nominal retention range of about 0.05, about 0.1, about 0.15 or about 0.2 micron.

In an embodiment, the solution is treated by a sterile filtration step wherein the filter has a nominal retention range of about 0.2 micron.

In an embodiment, the solution is treated by a sterile filtration step wherein the filter has a filter capacity of about 25-1500 L/m², 50-1500 L/m², 75-1500 L/m², 100-1500 L/m², 150-1500 L/m², 200-1500 L/m², 250-1500 L/m², 300-1500 L/m², 350-1500 L/m², 400-1500 L/m², 500-1500 L/m², 750-1500 L/m², 1000-1500 L/m² or 1250-1500 L/m².

In an embodiment, the solution is treated by a sterile filtration step wherein the filter has a filter capacity of about 25-1000 L/m², 50-1000 L/m², 75-1000 L/m², 100-1000 L/m², 150-1000 L/m², 200-1000 L/m², 250-1000 L/m², 300-1000 L/m², 350-1000 L/m², 400-1000 L/m², 500-1000 L/m² or 750-1000 L/m².

In an embodiment, the solution is treated by a sterile filtration step wherein the filter has a filter capacity of 25-500 L/m², 50-500 L/m², 75-500 L/m², 100-500 L/m², 150-500 L/m², 200-500 L/m², 250-500 L/m², 300-500 L/m², 350-500 L/m² or 400-500 L/m².

In an embodiment, the solution is treated by a sterile filtration step wherein the filter has a filter capacity of 25-300 L/m², 50-300 L/m², 75-300 L/m², 100-300 L/m², 150-300 L/m², 200-300 L/m² or 250-300 L/m².

In an embodiment, the solution is treated by a sterile filtration step wherein the filter has a filter capacity of 25-250 L/m², 50-250 L/m², 75-250 L/m², 100-250 L/m² or 150-250 L/m², 200-250 L/m².

In an embodiment, the solution is treated by a sterile filtration step wherein the filter has a filter capacity of 25-100 L/m², 50-100 L/m² or 75-100 L/m².

Any number within any of the above ranges is contemplated as an embodiment of the disclosure.

In an embodiment, the solution is treated by a microfiltration step wherein the filter has a filter capacity of about 25, about 50, about 75, about 100, about 150, about 200, about 250, about 300, about 350, about 400, about 500, about 600, about 700, about 800, about 900, about 1000, about 1100, about 1200, about 1300, about 1400 or about 1500 L/m².

1.12 Final Material

The polysaccharide can be finally prepared as a liquid solution

The polysaccharide can be further processed (e.g. lyophilized as a dried powder, see WO2006/110381). Therefore in an embodiment, the polysaccharide is a dried powder.

In an embodiment, the polysaccharide is a freeze-dried cake.

2 Uses of the Purified Polysaccharides

The polysaccharide purified by the method of the present invention may be used as antigens. Plain polysaccharides are used as antigens in vaccines (see the 23-valent unconjugated pneumococcal polysaccharide vaccine Pneumovax).

The polysaccharide purified by the method of the present invention may also be conjugated to carrier protein(s) to obtain a glycoconjugate.

2.1 Glycoconjugates

The polysaccharide purified by the method of the present invention may be conjugated to carrier protein(s) to obtain a glycoconjugate.

For the purposes of the invention the term ‘glycoconjugate’ indicates a saccharide covalently linked to a carrier protein. In one embodiment a saccharide is linked directly to a carrier protein. In a second embodiment a saccharide is linked to a carrier protein through a spacer/linker.

In general, covalent conjugation of saccharides to carriers enhances the immunogenicity of saccharides as it converts them from T-independent antigens to T-dependent antigens, thus allowing priming for immunological memory. Conjugation is particularly useful for pediatric vaccines.

Purified polysaccharides by the method of the invention may be activated (e.g., chemically activated) to make them capable of reacting (e.g. with a linker or directly with the carrier protein) and then incorporated into glycoconjugates, as further described herein.

The purified polysaccharide maybe sized to a target molecular weight before conjugation e.g. by the methods disclosed at section 1.11 above. Therefore in an embodiment, the purified polysaccharide is sized before conjugation. In an embodiment, the purified polysaccharide as disclosed herein may be sized before conjugation to obtain an oligosaccharide. Oligosaccharides have a low number of repeat units (typically 5-15 repeat units) and are typically derived by sizing (e.g. hydrolysis) of the polysaccharide.

Preferably though, the saccharide to be used for conjugation is a polysaccharide. High molecular weight polysaccharides are able to induce certain antibody immune responses due to the epitopes present on the antigenic surface. The isolation and purification of high molecular weight polysaccharides is preferably contemplated for use in the conjugates of the present invention.

Therefore in an embodiment, the polysaccharide is sized and remains a polysaccharide. In an embodiment, the polysaccharide is not sized.

In some embodiments, the purified polysaccharide before conjugation (after sizing or unsized) has a molecular weight of between 5 kDa and 4,000 kDa. In other such embodiments, the purified polysaccharide has a molecular weight of between 10 kDa and 4,000 kDa. In other such embodiments, the purified polysaccharide has a molecular weight of between 50 kDa and 4,000 kDa. In further such embodiments, the polysaccharide has a molecular weight of between 50 kDa and 3,500 kDa; between 50 kDa and 3,000 kDa; between 50 kDa and 2,500 kDa; between 50 kDa and 2,000 kDa; between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 4,000 kDa; between 100 kDa and 3,500 kDa; 100 kDa and 3,000 kDa; 100 kDa and 2,500 kDa; 100 kDa and 2,250 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 4,000 kDa; between 200 kDa and 3,500 kDa; between 200 kDa and 3,000 kDa; between 200 kDa and 2,500 kDa; between 200 kDa and 2,250 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. In further such embodiments, the polysaccharide has a molecular weight of between 250 kDa and 3,500 kDa; between 250 kDa and 3,000 kDa; between 250 kDa and 2,500 kDa; between 250 kDa and 2,000 kDa; between 250 kDa and 1,750 kDa; between 250 kDa and 1,500 kDa; between 250 kDa and 1,250 kDa; between 250 kDa and 1,000 kDa; between 250 kDa and 750 kDa; between 250 kDa and 500 kDa; between 300 kDa and 4,000 kDa; between 300 kDa and 3,500 kDa; 300 kDa and 3,000 kDa; 300 kDa and 2,500 kDa; 300 kDa and 2,250 kDa; between 300 kDa and 2,000 kDa; between 300 kDa and 1,750 kDa; between 300 kDa and 1,500 kDa; between 300 kDa and 1,250 kDa; between 300 kDa and 1,000 kDa; between 300 kDa and 750 kDa; between 300 kDa and 500 kDa; between 500 kDa and 4,000 kDa; between 500 kDa and 3,500 kDa; between 500 kDa and 3,000 kDa; between 500 kDa and 2,500 kDa; between 500 kDa and 2,250 kDa; between 500 kDa and 2,000 kDa; between 500 kDa and 1,750 kDa; between 500 kDa and 1,500 kDa; between 500 kDa and 1,250 kDa; between 500 kDa and 1,000 kDa; between 500 kDa and 750 kDa; or between 500 kDa and 600 kDa.

Any number within any of the above ranges is contemplated as an embodiment of the disclosure.

In some embodiments, the purified polysaccharide has a molecular weight of about 5 kDa, 10 kDa, 15 kDa, 20 kDa, 25 kDa, 30 kDa, 35 kDa, 40 kDa, 45 kDa, 50 kDa, 75 kDa, 90 kDa, 100 kDa, 150 kDa, 200 kDa, 250 kDa, 300 kDa, 350 kDa, 400 kDa, 450 kDa, 500 kDa, 550 kDa, 600 kDa, 650 kDa, 700 kDa, 750 kDa, 800 kDa, 850 kDa, 900 kDa, 950 kDa, 1000 kDa, 1250 kDa, 1500 kDa, 1750 kDa, 2000 kDa, 2250 kDa, 2500 kDa, 2750 kDa, 3000 kDa, 3250 kDa, 3500 kDa, 3750 kDa or 4,000 kDa.

In an embodiment, the purified polysaccharide is a capsular saccharide (polysaccharide or oligosaccharide).

In an embodiment, the purified polysaccharide is a capsular polysaccharide from Staphylococcus aureus. In an embodiment the purified polysaccharide is Staphylococcus aureus type 5 or type 8 capsular polysaccharide.

In a further embodiment, the purified polysaccharide is a capsular polysaccharide from Enterococcus faecalis. In yet a further embodiment, the purified polysaccharide is a capsular polysaccharide from Haemophilus influenzae type b.

In a further embodiment, the purified polysaccharide is a capsular polysaccharide from Neisseria meningitidis. In an embodiment the purified polysaccharide is a capsular polysaccharide from N. meningitidis serogroup A (MenA), N. meningitidis serogroup W135 (MenW135), N. meningitidis serogroup Y (MenY), N. meningitidis serogroup X (MenX) or N. meningitidis serogroup C (MenC).

In a further embodiment, the purified polysaccharide is a capsular polysaccharide from Escherichia coli. In an embodiment, the purified polysaccharide is a capsular polysaccharide from an Escherichia coli part of the Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), or Escherichia coli—enteroinvasive (EIEC). In an embodiment, the purified polysaccharide is a capsular polysaccharide from an Uropathogenic Escherichia coli (UPEC).

In an embodiment, the purified polysaccharide is a capsular polysaccharide from an Escherichia coli serotype selected from the group consisting of serotypes O157:H7, O26:H11, O111:H- and O103:H2. In an embodiment, the purified polysaccharide is a capsular polysaccharide from an Escherichia coli serotype selected from the group consisting of serotypes O6:K2:H1 and O18:K1:H7. In an embodiment, the purified polysaccharide is a capsular polysaccharide from an Escherichia coli serotype selected from the group consisting of serotypes O45:K1, O17:K52:H18, O19:H34 and O7:K1. In an embodiment, the purified polysaccharide is a capsular polysaccharide from an Escherichia coli serotype O104:H4. In an embodiment, the purified polysaccharide is a capsular polysaccharide from Escherichia coli serotype O1:K12:H7. In an embodiment, the purified polysaccharide is a capsular polysaccharide from an Escherichia coli serotype O127:H6. In an embodiment, the purified polysaccharide is a capsular polysaccharide from an Escherichia coli serotype O139:H28. In an embodiment, the purified polysaccharide is a capsular polysaccharide from an Escherichia coli serotype O128:H2.

In a further embodiment, the purified polysaccharide is a capsular polysaccharide from Streptococcus agalactiae (Group B Streptococcus (GBS)). In some embodiments, the purified polysaccharide is a capsular polysaccharide selected from the group consisting of GBS types Ia, Ib, II, III, IV, V, VI, VII and VIII capsular polysaccharides. In some embodiments, the purified polysaccharide is a capsular polysaccharide selected from the group consisting of GBS types Ia, Ib, II, III and V capsular polysaccharides. In a preferred embodiment, the purified polysaccharide is a capsular polysaccharide from Steptococcus pneumoniae. In an embodiment, the purified polysaccharide is the capsular polysaccharide from Streptococcus pneumoniae serotype 1. In a further embodiment, the purified polysaccharide is the capsular polysaccharide from Streptococcus pneumoniae serotype 2. In a further embodiment, the purified polysaccharide is the capsular polysaccharide from Streptococcus pneumoniae serotype 3. In a further embodiment, the purified polysaccharide is the capsular polysaccharide from Streptococcus pneumoniae serotype 4. In a further embodiment, the purified polysaccharide is the capsular polysaccharide from Streptococcus pneumoniae serotype 5. In a further embodiment, the purified polysaccharide is the capsular polysaccharide from Streptococcus pneumoniae serotype 6A. In a further embodiment, the purified polysaccharide is the capsular polysaccharide from Streptococcus pneumoniae serotype 6B. In a further embodiment, the purified polysaccharide is the capsular polysaccharide from Streptococcus pneumoniae serotype 6C. In a further embodiment, the purified polysaccharide is the capsular polysaccharide from Streptococcus pneumoniae serotype 7F. In a further embodiment, the purified polysaccharide is the capsular polysaccharide from Streptococcus pneumoniae serotype 8. In a further embodiment, the purified polysaccharide is the capsular polysaccharide from Streptococcus pneumoniae serotype 9V. In a further embodiment, the purified polysaccharide is the capsular polysaccharide from Streptococcus pneumoniae serotype 9N. In a further embodiment, the purified polysaccharide is the capsular polysaccharide from Streptococcus pneumoniae serotype 10A. In a further embodiment, the purified polysaccharide is the capsular polysaccharide from Streptococcus pneumoniae serotype 11A. In a further embodiment, the purified polysaccharide is the capsular polysaccharide from Streptococcus pneumoniae serotype 12F. In a further embodiment, the purified polysaccharide is the capsular polysaccharide from Streptococcus pneumoniae serotype 14. In a further embodiment, the purified polysaccharide is the capsular polysaccharide from Streptococcus pneumoniae serotype 15A. In a further embodiment, the purified polysaccharide is the capsular polysaccharide from Streptococcus pneumoniae serotype 15B. In a further embodiment, the purified polysaccharide is the capsular polysaccharide from Streptococcus pneumoniae serotype 15C. In a further embodiment, the purified polysaccharide is the capsular polysaccharide from Streptococcus pneumoniae serotype 16F. In a further embodiment, the purified polysaccharide is the capsular polysaccharide from Streptococcus pneumoniae serotype 17F. In a further embodiment, the purified polysaccharide is the capsular polysaccharide from Streptococcus pneumoniae serotype 18C. In a further embodiment, the purified polysaccharide is the capsular polysaccharide from Streptococcus pneumoniae serotype 19A. In a further embodiment, the purified polysaccharide is the capsular polysaccharide from Streptococcus pneumoniae serotype 19F. In a further embodiment, the purified polysaccharide is the capsular polysaccharide from Streptococcus pneumoniae serotype 20. In a further embodiment, the purified polysaccharide is the capsular polysaccharide from Streptococcus pneumoniae serotype 20A. In a further embodiment, the purified polysaccharide is the capsular polysaccharide from Streptococcus pneumoniae serotype 20B. In a further embodiment, the purified polysaccharide is the capsular polysaccharide from Streptococcus pneumoniae serotype 22F. In a further embodiment, the purified polysaccharide is the capsular polysaccharide from Streptococcus pneumoniae serotype 23A. In a further embodiment, the purified polysaccharide is the capsular polysaccharide from Streptococcus pneumoniae serotype 23B. In a further embodiment, the purified polysaccharide is the capsular polysaccharide from Streptococcus pneumoniae serotype 23F. In a further embodiment, the purified polysaccharide is the capsular polysaccharide from Streptococcus pneumoniae serotype 24B. In a further embodiment, the purified polysaccharide is the capsular polysaccharide from Streptococcus pneumoniae serotype 24F. In a further embodiment, the purified polysaccharide is the capsular polysaccharide from Streptococcus pneumoniae serotype 29. In a further embodiment, the purified polysaccharide is the capsular polysaccharide from Streptococcus pneumoniae serotype 31. In a further embodiment, the purified polysaccharide is the capsular polysaccharide from Streptococcus pneumoniae serotype 33F. In a further embodiment, the purified polysaccharide is the capsular polysaccharide from Streptococcus pneumoniae serotype 34. In a further embodiment, the purified polysaccharide is the capsular polysaccharide from Streptococcus pneumoniae serotype 35B. In a further embodiment, the purified polysaccharide is the capsular polysaccharide from Streptococcus pneumoniae serotype 35F. In a further embodiment, the purified polysaccharide is the capsular polysaccharide from Streptococcus pneumoniae serotype 38. In a further embodiment, the purified polysaccharide is the capsular polysaccharide from Streptococcus pneumoniae serotype 72. In a further embodiment, the purified polysaccharide is the capsular polysaccharide from Streptococcus pneumoniae serotype 73.

Any suitable conjugation reaction can be used, with any suitable linker where necessary. See for example WO2007116028 pages 17-22.

The purified oligosaccharides or polysaccharides described herein are chemically activated to make the saccharides capable of reacting with the carrier protein.

In an embodiment, the glycoconjugate is prepared using reductive amination.

Reductive amination involves two steps, (1) oxidation (activation) of the purified saccharide, (2) reduction of the activated saccharide and a carrier protein (e.g., CRM₁₉₇, DT, TT or PD) to form a glycoconjugate (see e.g. WO2015110941, WO2015110940). As mentioned above, before oxidation, sizing of the polysaccharide to a target molecular weight (MW) range can be performed. Mechanical or chemical hydrolysis may be employed. Chemical hydrolysis may be conducted using acetic acid. In an embodiment, the size of the purified polysaccharide is reduced by mechanical homogenization.

In an embodiment, the purified polysacharide or oligosaccharide is conjugated to a carrier protein by a process comprising the step of:

(a) reacting said purified polysaccharide or oligosaccharide with an oxidizing agent;

(b) optionally quenching the oxidation reaction by addition of a quenching agent;

(c) compounding the activated polysaccharide or oligosaccharide of step (a) or (b) with a carrier protein; and

(d) reacting the compounded activated polysaccharide or oligosaccharide and carrier protein with a reducing agent to form a glycoconjugate.

Following the oxidation step (a) the saccharide is said to be activated and is referred to as “activated polysaccharide or oligosaccharide”.

The oxidation step (a) may involve reaction with periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate).

In a preferred embodiment, the oxidizing agent is sodium periodate. In a an embodiment, the periodate used for the oxidation is metaperiodate. In an embodiment the periodate used for the oxidation is sodium metaperiodate.

The oxidation step (a) may involve reaction with a stable nitroxyl or nitroxide radical compound, such as piperidine-N-oxy or pyrrolidine-N-oxy compounds, in the presence of an oxidant to selectively oxidize primary hydroxyls of the said polysaccharide or oligosaccharide to produce an activated saccharide containing aldehyde groups (see WO2014097099). In an aspect, said stable nitroxyl or nitroxide radical compound is any one as disclosed at page 3 line 14 to page 4 line 7 of WO2014097099 and the oxidant is any one as disclosed at page 4 line 8 to 15 of WO2014097099. In an aspect, said stable nitroxyl or nitroxide radical compound is 2,2,6,6-tetramethyl-1-piperidinyloxy (TEMPO) and the oxidant is N-chlorosuccinimide (NCS).

In one embodiment, the quenching agent is as disclosed in WO2015110941 (see page 30 line 3 to 26).

In an embodiment, the reduction reaction (d) is carried out in aqueous solvent. In an embodiment, the reduction reaction (d) is carried out in aprotic solvent. In an embodiment, the reduction reaction (d) is carried out in DMSO (dimethylsulfoxide) or in DMF (dimethylformamide)) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In a preferred embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In an embodiment, the glycoconjugate is prepared using cyanylation chemistry.

In an embodiment, the purified polysaccharide or oligosaccharide is activated with cyanogen bromide. The activation corresponds to cyanylation of the hydroxyl groups of the polysaccharide or oligosaccharide. The activated polysaccharide or oligosaccharide is then coupled directly or via a spacer (linker) group to an amino group on the carrier protein.

In an embodiment, the purified polysaccharide or oligosaccharide is activated with 1-cyano-4-dimethylamino pyridinium tetrafluoroborate (CDAP) to form a cyanate ester. The activated polysaccharide or oligosaccharide is then coupled directly or via a spacer (linker) group to an amino group on the carrier protein.

In an embodiment, the spacer could be cystamine or cysteamine to give a thiolated polysaccharide or oligosaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA) or succinimidyl 3-[bromoacetamido]proprionate (SBAP)). Preferably, the cyanate ester (optionally made by CDAP chemistry) is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

In an embodiment, the glycoconjugate is prepared by using bis electrophilic reagents such as carbonyldiimidazole (CDI) or carbonylditriazole (CDT). In such an embodiment, the conjugation reaction is preferably made in aprotic solvents such as DMF or DMSO via a direct route or using bigeneric linkers (see e.g. WO2011041003).

In an embodiment, the glycoconjugate is prepared by a method of making glycoconjugates as disclosed in WO2014027302. The resulting glycoconjugate comprises a saccharide covalently conjugated to a carrier protein through a bivalent, heterobifunctional spacer (2-((2-oxoethyl)thio)ethyl)carbamate (eTEC). Alternatively, the the glycoconjugate is prepared by a method of making glycoconjugates as disclosed in WO2015121783.

Other suitable conjugation techniques use carbodiimides (e.g. EDC (1-Ethyl-3-(3-dimethylaminopropyl) carbodiimide hydrochloride, EDC plus Sulfo NHS, CMC (1-Cyclohexyl-3-(2-morpholinoethyl)carbodiimide, DCC (N,N′-Dicyclohexyl carbodiimide), or DIC (diisopropyl carbodiimide).

In an embodiment, the polysaccharide or oligosaccaride is conjugated to the carrier protein via a linker, for instance a bifunctional linker. The linker is optionally heterobifunctional or homobifunctional, having for example a reactive amino group and a reactive carboxylic acid group, 2 reactive amino groups or two reactive carboxylic acid groups. The linker has for example between 4 and 20, 4 and 12, 5 and 10 carbon atoms. A possible linker is adipic acid dihydrazide (ADH). Other linkers include B-propionamido (WO 00/10599), nitrophenyl-ethylamine, haloalkyl halide), glycosidic linkages (U.S. Pat. Nos. 4,673,574, 4,808,700), hexane diamine and 6-aminocaproic acid (U.S. Pat. No. 4,459,286).

Carrier Protein

A component of the glycoconjugate is a carrier protein to which the purified polysaccharide or oligosaccharide is conjugated. The terms “protein carrier” or “carrier protein” or “carrier” may be used interchangeably herein. Carrier proteins should be amenable to standard conjugation procedures.

In a preferred embodiment, the carrier protein of the glycoconjugate is selected in the group consisting of: DT (Diphtheria toxin), TT (tetanus toxid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRM₉, CRM₁₀₂, CRM₁₀₃ or CRM₁₀₇; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substution at glutamic acid 553 (Douglas et al. (1987) J. Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa.

In a preferred embodiment, the carrier protein of the glycoconjugate is independently selected from the group consisting of TT, DT, DT mutants (such as CRM₁₉₇), H. influenzae protein D, PhtX, PhtD, PhtDE fusions (particularly those described in WO 01/98334 and WO 03/054007), detoxified pneumolysin, PorB, N19 protein, PspA, OMPC, toxin A or B of C. difficile and PsaA.

In an embodiment, the carrier protein of the glycoconjugate is DT (Diphtheria toxoid). In another embodiment, the carrier protein of the glycoconjugate is TT (tetanus toxoid). In another embodiment, the carrier protein of the glycoconjugate is PD (H. influenzae protein D; see, e.g., EP0594610 B).

In a preferred embodiment, the purified polysaccharide or oligosaccharide is conjugated to CRM₁₉₇ protein. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{tox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the purified polysaccharide or oligosaccharide is conjugated to CRM₁₉₇ protein or the A chain of CRM₁₉₇ (see CN103495161). In an embodiment, the purified polysaccharide or oligosaccharide is conjugated the A chain of CRM₁₉₇ obtained via expression by genetically recombinant E. coli (see CN103495161).

Preferably the ratio of carrier protein to polysaccharide or oligosaccharide in the glycoconjugate is between 1:5 and 5:1; e.g. between 1:0.5 and 4:1, between 1:1 and 3.5:1, between 1.2:1 and 3:1, between 1.5:1 and 2.5:1; e.g. between 1:2 and 2.5:1 or between 1:1 and 2:1 (w/w). In an embodiment, the ratio of carrier protein to polysaccharide or oligosaccharide in the glycoconjugate is about 1:1, 1.1:1, 1.2:1, 1.3:1, 1.4:1, 1.5:1 or 1.6:1.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

Compositions may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

2.2 Immunogenic Compositions

In an embodiment the invention relates to an immunogenic composition comprising any of the purified polysaccharide and/or glycoconjugate disclosed herein.

In an embodiment the invention relates to an immunogenic composition comprising any of the glycoconjugate disclosed herein.

In an embodiment the invention relates to an immunogenic composition comprising from 1 to 25 different glycoconjugates disclosed at section 2.1.

In an embodiment the invention relates to an immunogenic composition comprising from 1 to 25 glycoconjugates from different serotypes of S. pneumoniae (1 to 25 pneumococcal conjugates). In one embodiment the invention relates to an immunogenic composition comprising glycoconjugates from 7, 8, 9, 10, 11, 12, 13, 14, 15, 16, 17, 18, 19, 20, 21, 22, 23, 24 or 25 different serotypes of S. pneumoniae. In one embodiment the immunogenic compositions comprises glycoconjugates from 16 or 20 different serotypes of S. pneumoniae. In an embodiment the immunogenic composition is a 7, 8, 9, 10, 11, 12, 13, 14, 15, 16, 17, 18, 19 or 20-valent pneumococcal conjugate compositions. In an embodiment the immunogenic composition is a 14, 15, 16, 17, 18 or 19-valent pneumococcal conjugate compositions. In an embodiment the immunogenic composition is a 16-valent pneumococcal conjugate composition. In an embodiment the immunogenic composition is a 19-valent pneumococcal conjugate compositions. In an embodiment the immunogenic composition is a 20-valent pneumococcal conjugate composition.

In an embodiment said immunogenic composition comprises glycoconjugates from S. pneumoniae serotypes 4, 6B, 9V, 14, 18C, 19F and 23F.

In an embodiment said immunogenic composition comprises in addition glycoconjugates from S. pneumoniae serotypes 1, 5 and 7F.

In an embodiment any of the immunogenic compositions above comprises in addition glycoconjugates from S. pneumoniae serotypes 6A and 19A.

In an embodiment any of the immunogenic compositions above comprise in addition a glycoconjugate from S. pneumoniae serotype 3.

In an embodiment any of the immunogenic compositions above comprise in addition a glycoconjugates from S. pneumoniae serotype 22F and 33F.

In an embodiment any of the immunogenic compositions above comprise in addition a glycoconjugates from S. pneumoniae serotypes 8, 10A, 11A, 12F and 15B.

In an embodiment any of the immunogenic compositions above comprise in addition a glycoconjugates from S. pneumoniae serotype 2.

In an embodiment any of the immunogenic compositions above comprise in addition a glycoconjugates from S. pneumoniae serotypes 9N.

In an embodiment any of the immunogenic compositions above comprise in addition a glycoconjugates from S. pneumoniae serotypes 17F.

In an embodiment any of the immunogenic compositions above comprise in addition a glycoconjugates from S. pneumoniae serotypes 20.

In an embodiment the immunogenic composition of the invention comprises glycoconjugates from S. pneumoniae serotypes 8, 10A, 11A, 12F, 15B, 22F and 33F.

In an embodiment any of the immunogenic compositions above comprise in addition a glycoconjugates from S. pneumoniae serotype 2.

In an embodiment any of the immunogenic compositions above comprise in addition a glycoconjugates from S. pneumoniae serotypes 9N.

In an embodiment any of the immunogenic compositions above comprise in addition a glycoconjugates from S. pneumoniae serotypes 17F.

In an embodiment any of the immunogenic compositions above comprise in addition a glycoconjugates from S. pneumoniae serotypes 20.

In a preferred embodiment though, the saccharides are each individually conjugated to different molecules of the protein carrier (each molecule of protein carrier only having one type of saccharide conjugated to it). In said embodiment, the capsular saccharides are said to be individually conjugated to the carrier protein. Preferably, all the glycoconjugates of the above immunogenic compositions are individually conjugated to the carrier protein.

In an embodiment of any of the above immunogenic compositions, the glycoconjugate from S. pneumoniae serotype 22F is conjugated to CRM₁₉₇. In an embodiment of any of the above immunogenic compositions, the glycoconjugate from S. pneumoniae serotype 33F is conjugated to CRM₁₉₇. In an embodiment of any of the above immunogenic compositions, the glycoconjugate from S. pneumoniae serotype 15B is conjugated to CRM 197. In an embodiment of any of the above immunogenic compositions, the glycoconjugate from S. pneumoniae serotype 12F is conjugated to CRM₁₉₇. In an embodiment of any of the above immunogenic compositions, the glycoconjugate from S. pneumoniae serotype 10A is conjugated to CRM₁₉₇. In an embodiment of any of the above immunogenic compositions, the glycoconjugate from S. pneumoniae serotype 11A is conjugated to CRM₁₉₇. In an embodiment of any of the above immunogenic compositions, the glycoconjugate from S. pneumoniae serotype 8 is conjugated to CRM₁₉₇. In an embodiment of any of the above immunogenic compositions, the glycoconjugates from S. pneumoniae serotypes 4, 6B, 9V, 14, 18C, 19F and 23F are conjugated to CRM₁₉₇. In an embodiment of any of the above immunogenic compositions, the glycoconjugates from S. pneumoniae serotypes 1, 5 and 7F are conjugated to CRM₁₉₇. In an embodiment of any of the above immunogenic compositions, the glycoconjugates from S. pneumoniae serotypes 6A and 19A are conjugated to CRM₁₉₇. In an embodiment of any of the above immunogenic compositions, the glycoconjugate from S. pneumoniae serotype 3 is conjugated to CRM₁₉₇.

In an embodiment, the glycoconjugates of any of the above immunogenic compositions are all individually conjugated to CRM₁₉₇.

In an embodiment, the glycoconjugates from S. pneumoniae serotypes 1, 4, 5, 6B, 7F, 9V, 14 and/or 23F of any of the above immunogenic compositions are individually conjugated to PD.

In an embodiment, the glycoconjugate from S. pneumoniae serotype 18C of any of the above immunogenic compositions is conjugated to TT.

In an embodiment, the glycoconjugate from S. pneumoniae serotype 19F of any of the above immunogenic compositions is conjugated to DT.

In an embodiment, the glycoconjugates from S. pneumoniae serotypes 1, 4, 5, 6B, 7F, 9V, 14 and/or 23F of any of the above immunogenic compositions are individually conjugated to PD, the glycoconjugate from S. pneumoniae serotype 18C is conjugated to TT and the glycoconjugate from S. pneumoniae serotype 19F is conjugated to DT.

In an embodiment the above immunogenic compositions comprise from 8 to 20 different serotypes of S. pneumoniae.

In an embodiment the invention relates to an immunogenic composition comprising from 1 to 5 glycoconjugates from different N. meningitidis serogroups (1 to 5 meningococcal conjugates). In one embodiment the invention relates to an immunogenic composition comprising glycoconjugates from 1, 2, 3, 4, or 5 different N. meningitidis serogroups. In one embodiment the immunogenic compositions comprises 4 or 5 different N. meningitidis. In an embodiment the immunogenic composition is a 1, 2, 3, 4 or 5-valent meningococcal conjugate compositions. In an embodiment the immunogenic composition is a 2-valent meningococcal conjugate composition. In an embodiment the immunogenic composition is a 4-valent meningococcal conjugate composition. In an embodiment the immunogenic composition is a 5-valent meningococcal conjugate composition.

In an embodiment the immunogenic composition comprises a conjugated N. meningitidis serogroup Y capsular saccharide (MenY), and/or a conjugated N. meningitidis serogroup C capsular saccharide (MenC).

In an embodiment the immunogenic composition comprises a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

In an embodiment the immunogenic compositions comprises a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

In an embodiment the immunogenic composition comprises a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), a conjugated N. meningitidis serogroup C capsular saccharide (MenC) and/or a conjugated N. meningitidis serogroup X capsular saccharide (MenX).

In some embodiments, the immunogenic compositions disclosed herein may further comprise at least one, two or three adjuvants. In some embodiments, the immunogenic compositions disclosed herein may further comprise one adjuvant. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In a preferred embodiment, the immunogenic compositions disclosed herein comprise aluminum phosphate or aluminum hydroxide as adjuvant.

Further exemplary adjuvants to enhance effectiveness of the immunogenic compositions as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalane, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, Mass.), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-s n-glycero-3-hydroxyphosphoryloxy)-ethylamine MTP-PE), etc.

In an embodiment of the present invention, the immunogenic compositions as disclosed herein comprise a CpG Oligonucleotide as adjuvant.

The immunogenic compositions may be formulated in liquid form (i.e., solutions or suspensions) or in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present disclosure can be accomplished using art-recognized methods. For instance, the individual polysaccharides and/or conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any of combination of polysaccahride or glycoconjugates disclosed herein and a pharmaceutically acceptable excipient, carrier, or diluent.

In an embodiment, the immunogenic composition of the disclosure is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the immunogenic compositions of the disclosure comprise a buffer.

In an embodiment, said buffer has a pKa of about 3.5 to about 7.5. In some embodiments, the buffer is phosphate, succinate, histidine or citrate. In certain embodiments, the buffer is succinate at a final concentration of 1 mM to 10 mM. In one particular embodiment, the final concentration of the succinate buffer is about 5 mM.

In an embodiment, the immunogenic compositions of the disclosure comprise a salt. In some embodiments, the salt is selected from the groups consisting of magnesium chloride, potassium chloride, sodium chloride and a combination thereof. In one particular embodiment, the salt is sodium chloride. In one particular embodiment, the immunogenic compositions of the invention comprise sodium chloride at 150 mM.

In an embodiment, the immunogenic compositions of the disclosure comprise a surfactant. In an embodiment, the surfactant is selected from the group consisting of polysorbate 20 (TWEEN™20), polysorbate 40 (TWEEN™40), polysorbate 60 (TWEEN™ 60), polysorbate 65 (TWEEN™ 65), polysorbate 80 (TWEEN™ 80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer. In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 1% polysorbate 80 (w/w).

In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w). In other embodiments, the final concentration of polysorbate 20 in the formulation is 0.01%, 0.02%, 0.03%, 0.04%, 0.05%, 0.06%, 0.07%, 0.08%, 0.09% or 0.1% polysorbate 20 (w/w). In another embodiment, the final concentration of the polysorbate 20 in the formulation is 1% polysorbate 20 (w/w).

In one particular embodiment, the surfactant is polysorbate 40. In some said embodiment, the final concentration of polysorbate 40 in the formulation is at least 0.0001% to 10% polysorbate 40 weight to weight (w/w). In some said embodiments, the final concentration of polysorbate 40 in the formulation is at least 0.001% to 1% polysorbate 40 weight to weight (w/w). In some said embodiments, the final concentration of polysorbate 40 in the formulation is at least 0.01% to 1% polysorbate 40 weight to weight (w/w). In other embodiments, the final concentration of polysorbate 40 in the formulation is 0.01%, 0.02%, 0.03%, 0.04%, 0.05%, 0.06%, 0.07%, 0.08%, 0.09% or 0.1% polysorbate 40 (w/w). In another embodiment, the final concentration of the polysorbate 40 in the formulation is 1% polysorbate 40 (w/w).

In one particular embodiment, the surfactant is polysorbate 60. In some said embodiment, the final concentration of polysorbate 60 in the formulation is at least 0.0001% to 10% polysorbate 60 weight to weight (w/w). In some said embodiments, the final concentration of polysorbate 60 in the formulation is at least 0.001% to 1% polysorbate 60 weight to weight (w/w). In some said embodiments, the final concentration of polysorbate 60 in the formulation is at least 0.01% to 1% polysorbate 60 weight to weight (w/w). In other embodiments, the final concentration of polysorbate 60 in the formulation is 0.01%, 0.02%, 0.03%, 0.04%, 0.05%, 0.06%, 0.07%, 0.08%, 0.09% or 0.1% polysorbate 60 (w/w). In another embodiment, the final concentration of the polysorbate 60 in the formulation is 1% polysorbate 60 (w/w).

In one particular embodiment, the surfactant is polysorbate 65. In some said embodiment, the final concentration of polysorbate 65 in the formulation is at least 0.0001% to 10% polysorbate 65 weight to weight (w/w). In some said embodiments, the final concentration of polysorbate 65 in the formulation is at least 0.001% to 1% polysorbate 65 weight to weight (w/w). In some said embodiments, the final concentration of polysorbate 65 in the formulation is at least 0.01% to 1% polysorbate 65 weight to weight (w/w). In other embodiments, the final concentration of polysorbate 65 in the formulation is 0.01%, 0.02%, 0.03%, 0.04%, 0.05%, 0.06%, 0.07%, 0.08%, 0.09% or 0.1% polysorbate 65 (w/w). In another embodiment, the final concentration of the polysorbate 65 in the formulation is 1% polysorbate 65 (w/w).

In one particular embodiment, the surfactant is polysorbate 85. In some said embodiment, the final concentration of polysorbate 85 in the formulation is at least 0.0001% to 10% polysorbate 85 weight to weight (w/w). In some said embodiments, the final concentration of polysorbate 85 in the formulation is at least 0.001% to 1% polysorbate 85 weight to weight (w/w). In some said embodiments, the final concentration of polysorbate 85 in the formulation is at least 0.01% to 1% polysorbate 85 weight to weight (w/w). In other embodiments, the final concentration of polysorbate 85 in the formulation is 0.01%, 0.02%, 0.03%, 0.04%, 0.05%, 0.06%, 0.07%, 0.08%, 0.09% or 0.1% polysorbate 85 (w/w). In another embodiment, the final concentration of the polysorbate 85 in the formulation is 1% polysorbate 85 (w/w).

In certain embodiments, the immunogenic composition of the disclosure has a pH of 5.5 to 7.5, more preferably a pH of 5.6 to 7.0, even more preferably a pH of 5.8 to 6.0.

In one embodiment, the present disclosure provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present disclosure is made of glass, metals (e.g., steel, stainless steel, aluminum, etc.) and/or polymers (e.g., thermoplastics, elastomers, thermoplastic-elastomers). In an embodiment, the container of the present disclosure is made of glass.

In one embodiment, the present disclosure provides a syringe filled with any of the immunogenic compositions disclosed herein. In certain embodiments, the syringe is siliconized and/or is made of glass.

A typical dose of the immunogenic composition of the invention for injection has a volume of 0.1 mL to 2 mL, more preferably 0.2 mL to 1 mL, even more preferably a volume of about 0.5 mL.

2.3 Use as Antigens

The polysaccharide purified by the method of the present invention ore the conjugates disclosed herein may be use as antigens. For example they may be part of a vaccine.

Therefore in an embodiment, the polysaccharides purified by the method of the present invention or the glycoconjugates obtained using said polysaccharides are for use in generating an immune response in a subject. In one aspect, the subject is a mammal, such as a human, cat, sheep, pig, horse, bovine or dog. In one aspect, the subject is a human.

In an embodiment, the polysaccharides purified by the method of the present invention, the glycoconjugates obtained using said polysaccharides or the immunogenic compositions disclosed herein are for use in a vaccine.

In an embodiment, the polysaccharides purified by the method of the present invention, the glycoconjugates obtained using said polysaccharides or the immunogenic compositions disclosed herein are for use as a medicament.

The immunogenic compositions described herein may be used in various therapeutic or prophylactic methods for preventing, treating or ameliorating a bacterial infection, disease or condition in a subject. In particular, immunogenic compositions described herein may be used to prevent, treat or ameliorate a S. pneumoniae, S. aureus, E. faecalis, Haemophilus influenzae type b, E. coli, Neisseria meningitidis or S. agalactiae infection, disease or condition in a subject.

Thus in one aspect, the disclosure provides a method of preventing, treating or ameliorating an infection, disease or condition associated with S. pneumoniae, S. aureus, E. faecalis, Haemophilus influenzae type b, E. coli, Neisseria meningitidis or S. agalactiae in a subject, comprising administering to the subject an immunologically effective amount of an immunogenic composition of the disclosure (in particular an immunogenic composition comprising the corresponding polysaccharide or glycoconjugate thereof).

In an embodiment, the disclosure provides a method of inducing an immune response to S. pneumoniae, S. aureus, E. faecalis, Haemophilus influenzae type b, E. coli, Neisseria meningitidis or S. agalactiae in a subject comprising administering to the subject an immunologically effective amount of an immunogenic composition of the disclosure (in particular an immunogenic composition comprising the corresponding polysaccharide or glycoconjugate thereof).

In an embodiment, the immunogenic compositions disclosed herein are for use as a vaccine. In such embodiments the immunogenic compositions described herein may be used to prevent S. pneumoniae, S. aureus, E. faecalis, Haemophilus influenzae type b, E. coli, Neisseria meningitidis or S. agalactiae infection in a subject. Thus in one aspect, the invention provides a method of preventing an infection by S. pneumoniae, S. aureus, E. faecalis, Haemophilus influenzae type b, E. coli, Neisseria meningitidis or S. agalactiae in a subject comprising administering to the subject an immunologically effective amount of an immunogenic composition of the disclosure.

In one aspect, the subject is a mammal, such as a human, cat, sheep, pig, horse, bovine or dog. In one aspect, the subject is a human.

The immunogenic compositions of the present disclosure can be used to protect or treat a human susceptible to a S. pneumoniae, S. aureus, E. faecalis, Haemophilus influenzae type b, E. coli, Neisseria meningitidis or S. agalactiae infection, by means of administering the immunogenic compositions via a systemic or mucosal route. In an embodiment, the immunogenic compositions disclosed herein are administered by intramuscular, intraperitoneal, intradermal or subcutaneous routes. In an embodiment, the immunogenic compositions disclosed herein are administered by intramuscular, intraperitoneal, intradermal or subcutaneous injection. In an embodiment, the immunogenic compositions disclosed herein are administered by intramuscular or subcutaneous injection.

In some cases, as little as one dose of the immunogenic composition according to the disclosure is needed, but under some circumstances, such as conditions of greater immune deficiency, a second, third or fourth dose may be given. Following an initial vaccination, subjects can receive one or several booster immunizations adequately spaced.

In an embodiment, the schedule of vaccination of the immunogenic composition according to the disclosure is a single dose.

In an embodiment, the schedule of vaccination of the immunogenic composition according to the disclosure is a multiple dose schedule.

3 Particular embodiments of the invention are set forth in the following numbered paragraphs:
1. A method for purifying a bacterial polysaccharide from a solution comprising said polysaccharide together with contaminants, wherein said method comprises a flocculation step.
2. The method of paragraph 1 wherein the flocculating agent comprises a multivalent cation.
3. The method of paragraph 2 wherein said multivalent cation is selected from the group consisting of aluminium, iron, calcium and magnesium.
4. The method of paragraph 2 wherein said flocculating agent is a mixture of at least two multivalent cations selected from the group consisting of aluminium, iron, calcium and magnesium.
5. The method of paragraph 2 wherein said flocculating agent is a mixture of at least three multivalent cations selected from the group consisting of aluminium, iron, calcium and magnesium.
6. The method of paragraph 2 wherein said flocculating agent is a mixture of four multivalent cations consisting of aluminium, iron, calcium and magnesium.
7. The method of paragraph 1 wherein the flocculating agent comprises an agent selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate.
8. The method of paragraph 1 wherein the flocculating agent is selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.
9. The method of paragraph 1 wherein the flocculating agent is polyethylenimine (PEI).
10. The method of paragraph 1 wherein the flocculating agent comprises alum.
11. The method of paragraph 1 wherein the flocculating agent is alum.
12. The method of paragraph 1 wherein the flocculating agent comprises potassium alum.
13. The method of paragraph 1 wherein the flocculating agent potassium alum.
14. The method of paragraph 1 wherein the flocculating agent comprises sodium alum.
15. The method of paragraph 1 wherein the flocculating agent is sodium alum.
16. The method of paragraph 1 wherein the flocculating agent comprises ammonium alum.
17. The method of paragraph 1 wherein the flocculating agent is ammonium alum.
18. The method of paragraph 1 wherein the flocculating agent is a mixture of two agents selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate. In an embodiment, the flocculating agent is selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.
19. The method of paragraph 1 wherein the flocculating agent is a mixture of three agents selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate.
20. The method of paragraph 1 wherein the flocculating agent is a mixture of four agents selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.
21. The method of paragraph 1 wherein the flocculating agent comprises an agent selected from the group consisting of chitosan, isinglass, moringa oleifera seeds (Horseradish Tree), gelatin, strychnos potatorum seeds (Nirmali nut tree), guar gum and alginates (e.g. brown seaweed extracts). In an embodiment, the flocculating agent is selected from the group consisting of chitosan, isinglass, moringa oleifera seeds (Horseradish Tree), gelatin, strychnos potatorum seeds (Nirmali nut tree), guar gum and alginates (e.g. brown seaweed extracts).
22. The method of paragraph 1 wherein the flocculating agent is an agent selected from the group consisting of chitosan, isinglass, moringa oleifera seeds (Horseradish Tree), gelatin, strychnos potatorum seeds (Nirmali nut tree), guar gum and alginates (e.g. brown seaweed extracts). In an embodiment, the flocculating agent is selected from the group consisting of chitosan, isinglass, moringa oleifera seeds (Horseradish Tree), gelatin, strychnos potatorum seeds (Nirmali nut tree), guar gum and alginates (e.g. brown seaweed extracts).
23. The method of any one of paragraphs 1-22 wherein the concentration of flocculating agent is between about 0.1 and about 20% (w/v).
24. The method of any one of paragraphs 1-22 wherein the concentration of flocculating agent is between about 0.5 and about 10% (w/v).
25. The method of any one of paragraphs 1-22 wherein the concentration of flocculating agent is between about 1 and about 5% (w/v).
26. The method of any one of paragraphs 1-22 wherein the concentration of flocculating agent is about 0.1, about 0.25, about 0.5, about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10% (w/v).
27. The method of any one of paragraphs 1-22 wherein the concentration of flocculating agent is about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5 or about 20.0% (w/v)
28. The method of any one of paragraphs 1-22 wherein the concentration of flocculating agent is about 0.5, about 1.0, about 1.5, about 2.0, about 2.5, about 3.0, about 3.5, about 4.0, about 4.5 or about 5.0% (w/v)
29. The method of any one of paragraphs 1-22 wherein the concentration of flocculating agent is about 1.0, about 1.5, about 2.0, about 2.5, about 3.0, about 3.5 or about 4.0% (w/v) is used.
30. The method of any one of paragraphs 1-29 wherein the flocculating agent is added over a period of between a few seconds (e.g. 1 to 10 seconds) and about one month.
31. The method of any one of paragraphs 1-29 wherein the flocculating agent is added over a period between about 2 seconds and about two weeks.
32. The method of any one of paragraphs 1-29 wherein the flocculating agent is added over a period of between about 1 minute and about one week.
33. The method of any one of paragraphs 1-29 wherein the flocculating agent is added over a period of between about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours and about two days.
34. The method of any one of paragraphs 1-29 wherein the flocculating agent is added over a period of between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.
35. The method of any one of paragraphs 1-29 wherein the flocculating agent is added over a period of between about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.
36. The method of any one of paragraphs 1-29 wherein the flocculating agent is added over a period of between about 15 minutes and about 3 hours.
37. The method of any one of paragraphs 1-29 wherein the flocculating agent is added over a period of between about 30 minutes and about 120 minutes.
38. The method of any one of paragraphs 1-29 wherein the flocculating agent is added over a period of about 2 seconds, about 10 seconds, about 30 seconds, about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 170 minutes, about 3.0 hours, about 3.5 hours, about 4.0 hours, about 4.5 hours, about 5.0 hours, about 5.5 hours, about 6.0 hours, about 6.5 hours, about 7.0 hours, about 7.5 hours, about 8.0 hours, about 8.5 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, about 48 hours, about 3 days, about 4 days, about 5 days, about 6 days, about 7 days, about 8 days, about 9 days, about 10 days, about 11 days, about 12 days, about 13 days, about 14 days or about 15 days.
39. The method of any one of paragraphs 1-38 wherein the flocculating agent is added without agitation.
40. The method of any one of paragraphs 1-38 wherein the flocculating agent is added under agitation.
41. The method of any one of paragraphs 1-38 wherein the flocculating agent is added under gentle agitation.
42. The method of any one of paragraphs 1-38 wherein the flocculating agent is added under vigorous agitation.
43. The method of any one of paragraphs 1-42 wherein the solution is hold for some time to allow settling of the flocs prior to downstream processing.
44. The method of any one of paragraphs 1-43 wherein the flocculation step is performed with a settling time of between a few seconds (e.g. 2 to 10 seconds) to about 1 minute.
45. The method of any one of paragraphs 1-43 wherein the flocculation step is performed with a settling time of at least about 2, at least about 3, at least about 4, at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 115, at least about 120, at least about 125, at least about 130, at least about 135, at least about 140, at least about 145, at least about 150, at least about 155 or at least about 160 minutes.
46. The method of paragraph 1-43 wherein the settling time is less than a week.
47. The method of any one of paragraphs 1-43 wherein the flocculation step is performed with a settling time of between about 1, about 2, about 3, about 4, about 5, about 6, about 7, about 8, about 9, about 10, about 15, about 20, about 25, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380, about 1440 minute(s), about two days, about three days, about four days, about five days or about six days and 1 week.
48. The method of any one of paragraphs 1-43 wherein the flocculation step is performed with a settling time of between a few seconds (e.g. 1 to 10 seconds) and about one month.
49. The method of any one of paragraphs 1-43 wherein the flocculation step is performed with a settling time of between about 2 seconds and about two weeks.
50. The method of any one of paragraphs 1-43 wherein the flocculation step is performed with a settling time of between about 1 minute and about one week.
51. The method of any one of paragraphs 1-43 wherein the flocculation step is performed with a settling time of between about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours and about two days.
52. The method of any one of paragraphs 1-43 wherein the flocculation step is performed with a settling time of between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.
53. The method of any one of paragraphs 1-43 wherein the flocculation step is performed with a settling time of between about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.
54. The method of any one of paragraphs 1-43 wherein the flocculation step is performed with a settling time of between about 15 minutes and about 3 hours.
55. The method of any one of paragraphs 1-43 wherein the flocculation step is performed with a settling time of between about 30 minutes and about 120 minutes.
56. The method of any one of paragraphs 1-43 wherein the flocculation step is performed with a settling time of about 10 seconds, about 30 seconds, about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 170 minutes, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, about 48 hours, about 3 days, about 4 days, about 5 days, about 6 days, about 7 days, about 8 days, about 9 days, about 10 days, about 11 days, about 12 days, about 13 days, about 14 days or about 15 days.
57. The method of any one of paragraphs 1-43 wherein the flocculation step is performed with a settling time of about 5, about 10, about 15, about 20, about 25, about 30, about 60, about 90, about 120, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380 or about 1440 minute(s) and two days.
58. The method of any one of paragraphs 1-43 wherein the flocculation step is performed with a settling time of between about 5 minutes and about one day.
59. The method of any one of paragraphs 1-43 wherein the flocculation step is performed with a settling time of between about 5 minutes and about 120 minutes.
60. The method of any one of paragraphs 1-43 wherein the flocculation step is performed with a settling time of about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes or about 160 minutes.
61. The method of any one of paragraphs 43-60 wherein the settling step is conducted without agitation.
62. The method of any one of paragraphs 43-60 wherein the settling step is conducted under agitation.
63. The method of any one of paragraphs 43-60 wherein the settling step is conducted under gentle agitation.
64. The method of any one of paragraphs 43-60 wherein the settling step is conducted under vigorous agitation.
65. The method of any one of paragraphs 1-64 wherein said flocculation step is performed at an acidic pH.
66. The method of any one of paragraphs 1-64 wherein said flocculation step is performed at a pH below 7.0, 6.0, 5.0 or 4.0.
67. The method of any one of paragraphs 1-64 wherein said flocculation step is performed at a pH between 7.0 and 1.0.
68. The method of any one of paragraphs 1-64 wherein said flocculation step is performed at a pH between 5.5 and 2.5, 5.0 and 2.5, 4.5 and 2.5, 4.0 and 2.5, 5.5 and 3.0, 5.0 and 3.0, 4.5 and 3.0, 4.0 and 3.0, 5.5 and 3.5, 5.0 and 3.5, 4.5 and 3.5 or 4.0 and 3.5.
69. The method of any one of paragraphs 1-64 wherein said flocculation step is performed at a pH of about 5.5, about 5.0, about 4.5, about 4.0, about 3.5, about 3.0, about 2.5, about 2.0, about 1.5 or about 1.0.
70. The method of any one of paragraphs 1-64 wherein said flocculation step is performed at a pH of about 4.0, about 3.5, about 3.0 or about 2.5.
71. The method of any one of paragraphs 1-64 wherein said flocculation step is performed at a pH of about 3.5.
72. The method of any one of paragraphs 65-71 wherein said acidic pH is obtained by acidifying the solution with an acid.
73. The method of any one of paragraphs 65-71 wherein said acidic pH is obtained by acidifying the solution with an acid selected from the group consisting of HCl, H₃PO₄, citric acid, acetic acid, nitrous acid, and sulfuric acid.
74. The method of any one of paragraphs 65-71 wherein said acidic pH is obtained by acidifying the solution with an amino acid.
75. The method of any one of paragraphs 65-71 wherein said acidic pH is obtained by acidifying the solution with an amino acid selected from the group consisting of glycine, alanine and glutamate.
76. The method of any one of paragraphs 65-71 wherein said acidic pH is obtained by acidifying the solution with sulfuric acid.
77. The method of any one of paragraphs 65-71 wherein the acid is added under agitation.
78. The method of any one of paragraphs 65-71 wherein the acid is added under gentle agitation.
79. The method of any one of paragraphs 65-71 wherein the acid is added under vigorous agitation.
80. The method of any one of paragraphs 1-79 wherein the addition of the flocculating agent is performed at a temperature between about 4° C. and about 30° C.
81. The method of any one of paragraphs 1-79 wherein the addition of the flocculating agent is performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
82. The method of any one of paragraphs 1-79 wherein the addition of the flocculating agent is performed at a temperature of about 20° C.
83. The method of any one of paragraphs 1-79 wherein the addition of the flocculating agent is performed at a temperature of between about 30° C. to about 95° C.
84. The method of any one of paragraphs 1-79 wherein the addition of the flocculating agent is performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
85. The method of any one of paragraphs 1-79 wherein the addition of the flocculating agent is performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
86. The method of any one of paragraphs 1-79 wherein the addition of the flocculating agent is performed at a temperature of about 50° C.
87. The method of any one of paragraphs 43-86 wherein the settling step, if present, is performed at a temperature between about 4° C. and about 30° C.
88. The method of any one of paragraphs 43-86 wherein the settling step, if present, is performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
89. The method of any one of paragraphs 43-86 wherein the settling step, if present, is performed at a temperature of about 20° C.
90. The method of any one of paragraphs 43-86 wherein the settling step, if present, is performed at a temperature of between about 30° C. to about 95° C.
91. The method of any one of paragraphs 43-86 wherein the settling step, if present, is performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
92. The method of any one of paragraphs 43-86 wherein the settling step, if present, is performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
93. The method of any one of paragraphs 43-86 wherein the settling step, if present, is performed at a temperature of about 50° C.
94. The method of any one of paragraphs 72-93 wherein the acidification step, if present, is performed at a temperature between about 4° C. and about 30° C.
95. The method of any one of paragraphs 72-93 wherein the acidification step, if present, is performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
96. The method of any one of paragraphs 72-93 wherein the acidification step, if present, is performed at a temperature of about 20° C.
97. The method of any one of paragraphs 72-93 wherein the acidification step, if present, is performed at a temperature of between about 30° C. to about 95° C.
98. The method of any one of paragraphs 72-93 wherein the acidification step, if present, is performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
99. The method of any one of paragraphs 72-93 wherein the acidification step, if present, is performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
100. The method of any one of paragraphs 72-93 wherein the acidification step, if present, is performed at a temperature of about 50° C.
101. The method of any one of paragraphs 1-79 wherein the addition of the flocculating agent and the settling step, if present, are performed at a temperature between about 4° C. and about 30° C.
102. The method of any one of paragraphs 1-79 wherein the addition of the flocculating agent and the settling step, if present, are performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
103. The method of any one of paragraphs 1-79 wherein the addition of the flocculating agent and the settling step, if present, are performed at a temperature of about 20° C.
104. The method of any one of paragraphs 1-79 wherein the addition of the flocculating agent and the settling step, if present, are performed at a temperature of between about 30° C. to about 95° C.
105. The method of any one of paragraphs 1-79 wherein the addition of the flocculating agent and the settling step, if present, are performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
106. The method of any one of paragraphs 1-79 wherein the addition of the flocculating agent and the settling step, if present, are performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
107. The method of any one of paragraphs 1-79 wherein the addition of the flocculating agent and the settling step, if present, are performed at a temperature of about 50° C.
108. The method of any one of 72-79 wherein the addition of the flocculating agent and the acidification step are performed at a temperature between about 4° C. and about 30° C.
109. The method of any one of paragraphs 72-79 wherein the addition of the flocculating agent and the acidification step are performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
110. The method of any one of paragraphs 72-79 wherein the addition of the flocculating agent and the acidification step are performed at a temperature of about 20° C.
111. The method of any one of paragraphs 72-79 wherein the addition of the flocculating agent and the acidification step are performed at a temperature of between about 30° C. to about 95° C.
112. The method of any one of paragraphs 72-79 wherein the addition of the flocculating agent and the acidification step are performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
113. The method of any one of paragraphs 72-79 wherein the addition of the flocculating agent and the acidification step are performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
114. The method of any one of paragraphs 72-79 wherein the addition of the flocculating agent and the acidification step are performed at a temperature of about 50° C.
115. The method of any one of paragraphs 72-79 wherein the addition of the flocculating agent, the settling and acidification steps are performed at a temperature between about 4° C. and about 30° C.
116. The method of any one of paragraphs 72-79 wherein the addition of the flocculating agent, the settling and acidification steps are performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
117. The method of any one of paragraphs 72-79 wherein the addition of the flocculating agent, the settling and acidification steps are performed at a temperature of about 20° C.
118. The method of any one of paragraphs 72-79 wherein the addition of the flocculating agent, the settling and acidification steps are performed at a temperature of between about 30° C. to about 95° C.
119. The method of any one of paragraphs 72-79 wherein the addition of the flocculating agent, the settling and acidification steps are performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
120. The method of any one of paragraphs 72-79 wherein the addition of the flocculating agent, the settling and acidification steps are performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
121. The method of any one of paragraphs 72-79 wherein the addition of the flocculating agent, the settling and acidification steps are performed at a temperature of about 50° C.
122. The method of any one of paragraphs 1-71, 80-93 or 101-107 wherein the flocculation step comprises adding a flocculating agent without pH adjustment.
123. The method of any one of paragraphs 1-122 wherein the flocculation step comprises adding a flocculating agent, adjusting the pH and settling the solution.
124. The method of paragraph 123 wherein, the flocculating agent is added before adjusting the pH.
125. The method of paragraph 123 wherein, the pH is adjusted before adding the flocculating agent.
126. The method of paragraph 123 wherein, the pH is adjusted before adding the flocculating agent and settling the solution.
127. The method of paragraph 123 wherein, the flocculating agent is added and the solution is settled before adjusting the pH.
128. The method of any one of paragraphs 1-127 wherein, following flocculation the suspension is clarified by decantation, sedimentation, filtration or centrifugation.
129. The method of any one of paragraphs 1-127 wherein, following flocculation the suspension is clarified by decantation.
130. The method of any one of paragraphs 1-127 wherein, following flocculation the suspension is clarified by hydrocyclone.
131. The method of any one of paragraphs 1-127 wherein, following flocculation the suspension is clarified by sedimentation.
132. The method of any one of paragraphs 1-127 wherein, following flocculation the suspension is clarified by flotation.
133. The method of any one of paragraphs 1-127 wherein, following flocculation the suspension is clarified by filtration
134. The method of any one of paragraphs 1-127 wherein, following flocculation the suspension is clarified by centrifugation.
135. The method of any one of paragraphs 127-134 wherein, the polysaccharide-containing solution is collected for storage.
136. The method of any one of paragraphs 127-134 wherein, the polysaccharide-containing solution is collected for additional processing.
137. The method of any one of paragraphs 127-134 wherein, the polysaccharide-containing solution is stored and then additionally processed.
138. The method of any one of paragraphs 134-137 wherein, said centrifugation is continuous centrifugation.
139. The method of any one of paragraphs 134-137 wherein, said centrifugation is bucket centrifugation.
140. The method of any one of paragraphs 134-139 wherein, the suspension is centrifuged at about 1,000 g, about 2,000 g, about 3,000 g, about 4,000 g, about 5,000 g, about 6,000 g, about 8,000 g, about 9,000 g, about 10,000 g, about 11,000 g, about 12,000 g, about 13,000 g, about 14,000 g, about 15,000 g, about 16,000 g, about 17,000 g, about 18,000 g, about 19,000 g, about 20,000 g, about 25,000 g, about 30,000 g, about 35,000 g, about 40,000 g, about 50,000 g, about 60,000 g, about 70,000 g, about 80,000 g, about 90,000 g, about 100,000 g, about 120,000 g, about 140,000 g, about 160,000 g or about 180,000 g.
141. The method of any one of paragraphs 134-139 wherein, the suspension is centrifuged at about 8,000 g, about 9,000 g, about 10,000 g, about 11,000 g, about 12,000 g, about 13,000 g, about 14,000 g, about 15,000 g, about 16,000 g, about 17,000 g, about 18,000 g, about 19,000 g, about 20,000 g or about 25,000 g.
142. The method of any one of paragraphs 134-139 wherein, the suspension is centrifuged between about 5,000 g and about 25,000 g.
143. The method of any one of paragraphs 134-139 wherein, the suspension is centrifuged between about 8,000 g and about 20,000 g.
144. The method of any one of paragraphs 134-139 wherein, the suspension is centrifuged between about 10,000 g and about 15,000 g.
145. The method of any one of paragraphs 134-139 wherein, the suspension is centrifuged between about 10,000 g and about 12,000 g.
146. The method of any one of paragraphs 134-145 wherein, the suspension is centrifuged during at least 2, at least 3, at least 4, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155 or at least 160 minutes.
147. The method of any one of paragraph 146 wherein, the suspension is centrifuged during less than 24 hours.
148. The method of any one of paragraphs 134-145 wherein, the suspension is centrifuged during between about 5, about 10, about 15, about 20, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320 or about 1380 minutes and 1440 minutes.
149. Preferably the suspension is centrifuged during between about 5, about 10, about 15, about 20, about 25, about 30, about 60, about 90, about 120, about 180, about 240, about 300, about 360, about 420, about 480 or about 540 minutes and about 600 minutes.
150. The method of any one of paragraphs 134-145 wherein, the suspension is centrifuged during between about 5 minutes and about 3 hours.
151. The method of any one of paragraphs 134-145 wherein, the suspension is centrifuged during between about 5 minutes and about 120 minutes.
152. The method of any one of paragraphs 134-145 wherein, the suspension is centrifuged during between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes or about 155 minutes and about 160 minutes.
153. The method of any one of paragraphs 134-145 wherein, the suspension is centrifuged during between about 10 minutes, about 15 minutes, about 20 minutes, about 25 minutes, about 30 minutes, about 35 minutes, about 40 minutes, about 45 minutes, about 50 minutes or about 55 minutes and about 60 minutes.
154. The method of any one of paragraphs 134-145 wherein, the suspension is centrifuged during about 5, about 10, about 15, about 20, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380 minutes or about 1440 minutes.
155. The method of any one of paragraphs 134-145 wherein, the suspension is centrifuged during about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes or about 160 minutes.
156. The method of any one of paragraphs 134-138 or 140-155 wherein, said centrifugation is continuous centrifugation and the feed rate is between 50-5000 ml/min, 100-4000 ml/min, 150-3000 ml/min, 200-2500 ml/min, 250-2000 ml/min, 300-1500 ml/min, 300-1000 ml/min, 200-1000 ml/min, 200-1500 ml/min, 400-1500 ml/min, 500-1500 ml/min, 500-1000 ml/min, 500-2000 ml/min, 500-2500 ml/min or 1000-2500 ml/min.
157. The method of any one of paragraphs 134-138 or 140-155 wherein, said centrifugation is continuous centrifugation and the feed rate is about 10, about 25, about 50, about 75, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1650 about 1700, about 1800, about 1900, about 2000, about 2100, about 2200, about 2300, about 2400, about 2500, about 2600, about 2700, about 2800, about 2900, about 3000, about 3250, about 3500, about 3750 about 4000, about 4250, about 4500 or about 5000 ml/min.
158. The method of any one of paragraphs 1-157 wherein, the polysaccharide containing solution is filtrated.
159. The method of paragraph 158 wherein, said filtration is selected from the group consisting of depth filtration, filtration through activated carbon, size filtration, diafiltration and ultrafiltration.
160. The method of paragraph 158 wherein, said filtration step is diafiltration.
161. The method of paragraph 160 wherein, said filtration is tangential flow filtration.
162. The method of paragraph 158 wherein, said filtration is depth filtration.
163. The method of paragraph 162 wherein, wherein the depth filter design is selected from the group consisting of cassettes, cartridges, deep bed (e.g. sand filter) and lenticular filters.
164. The method of any one of paragraphs 158-159 or 162-163 wherein the depth filter has a nominal retention range of between about 0.01-100 micron, about 0.05-100 micron, about 0.1-100 micron, about 0.2-100 micron, about 0.3-100 micron, about 0.4-100 micron, about 0.5-100 micron, about 0.6-100 micron, about 0.7-100 micron, about 0.8-100 micron, about 0.9-100 micron, about 1-100 micron, about 1.25-100 micron, about 1.5-100 micron, about 1.75-100 micron, about 2-100 micron, about 3-100 micron, about 4-100 micron, about 5-100 micron, about 6-100 micron, about 7-100 micron, about 8-100 micron, about 9-100 micron, about 10-100 micron, about 15-100 micron, about 20-100 micron, about 25-100 micron, about 30-100 micron, about 40-100 micron, about 50-100 micron or about 75-100 micron.
165. The method of any one of paragraphs 158-159 or 162-163 wherein the depth filter has a nominal retention range of between about 0.01-75 micron, about 0.05-75 micron, about 0.1-75 micron, about 0.2-75 micron, about 0.3-75 micron, about 0.4-75 micron, about 0.5-75 micron, about 0.6-75 micron, about 0.7-75 micron, about 0.8-75 micron, about 0.9-75 micron, about 1-75 micron, about 1.25-75 micron, about 1.5-75 micron, about 1.75-75 micron, about 2-75 micron, about 3-75 micron, about 4-75 micron, about 5-75 micron, about 6-75 micron, about 7-75 micron, about 8-75 micron, about 9-75 micron, about 10-75 micron, about 15-75 micron, about 20-75 micron, about 25-75 micron, about 30-75 micron, about 40-75 micron or about 50-75 micron.
166. The method of any one of paragraphs 158-159 or 162-163 wherein the depth filter has a nominal retention range of between about 0.01-50 micron, about 0.05-50 micron, about 0.1-50 micron, about 0.2-50 micron, about 0.3-50 micron, about 0.4-50 micron, about 0.5-50 micron, about 0.6-50 micron, about 0.7-50 micron, about 0.8-50 micron, about 0.9-50 micron, about 1-50 micron, about 1.25-50 micron, about 1.5-50 micron, about 1.75-50 micron, about 2-50 micron, about 3-50 micron, about 4-50 micron, about 5-50 micron, about 6-50 micron, about 7-50 micron, about 8-50 micron, about 9-50 micron, about 10-50 micron, about 15-50 micron, about 20-50 micron, about 25-50 micron, about 30-50 micron, about 40-50 micron or about 50-50 micron.
167. The method of any one of paragraphs 158-159 or 162-163 wherein the depth filter has a nominal retention range of between about 0.01-25 micron, about 0.05-25 micron, about 0.1-25 micron, about 0.2-25 micron, about 0.3-25 micron, about 0.4-25 micron, about 0.5-25 micron, about 0.6-25 micron, about 0.7-25 micron, about 0.8-25 micron, about 0.9-25 micron, about 1-25 micron, about 1.25-25 micron, about 1.5-25 micron, about 1.75-25 micron, about 2-25 micron, about 3-25 micron, about 4-25 micron, about 5-25 micron, about 6-25 micron, about 7-25 micron, about 8-25 micron, about 9-25 micron, about 10-25 micron, about 15-25 micron or about 20-25 micron.
168. The method of any one of paragraphs 158-159 or 162-163 wherein the depth filter has a nominal retention range of between about 0.01-10 micron, about 0.05-10 micron, about 0.1-10 micron, about 0.2-10 micron, about 0.3-10 micron, about 0.4-10 micron, about 0.5-10 micron, about 0.6-10 micron, about 0.7-10 micron, about 0.8-10 micron, about 0.9-10 micron, about 1-10 micron, about 1.25-10 micron, about 1.5-10 micron, about 1.75-10 micron, about 2-10 micron, about 3-10 micron, about 4-10 micron, about 5-10 micron, about 6-10 micron, about 7-10 micron, about 8-10 micron or about 9-10 micron.
169. The method of any one of paragraphs 158-159 or 162-163 wherein the depth filter has a nominal retention range of between about 0.01-8 micron, about 0.05-8 micron, about 0.1-8 micron, about 0.2-8 micron, about 0.3-8 micron, about 0.4-8 micron, about 0.5-8 micron, about 0.6-8 micron, about 0.7-8 micron, about 0.8-8 micron, about 0.9-8 micron, about 1-8 micron, about 1.25-8 micron, about 1.5-8 micron, about 1.75-8 micron, about 2-8 micron, about 3-8 micron, about 4-8 micron, about 5-8 micron, about 6-8 micron or about 7-8 micron.
170. The method of any one of paragraphs 158-159 or 162-163 wherein the depth filter has a nominal retention range of between about 0.01-5 micron, about 0.05-5 micron, about 0.1-5 micron, about 0.2-5 micron, about 0.3-5 micron, about 0.4-5 micron, about 0.5-5 micron, about 0.6-5 micron, about 0.7-5 micron, about 0.8-5 micron, about 0.9-5 micron, about 1-5 micron, about 1.25-5 micron, about 1.5-5 micron, about 1.75-5 micron, about 2-5 micron, about 3-5 micron or about 4-5 micron.
171. The method of any one of paragraphs 158-159 or 162-163 wherein the depth filter has a nominal retention range of between about 0.01-2 micron, about 0.05-2 micron, about 0.1-2 micron, about 0.2-2 micron, about 0.3-2 micron, about 0.4-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, about 1.75-2 micron, about 2-2 micron, about 3-2 micron or about 4-2 micron.
172. The method of any one of paragraphs 158-159 or 162-163 wherein the depth filter has a nominal retention range of between about 0.01-1 micron, about 0.05-1 micron, about 0.1-1 micron, about 0.2-1 micron, about 0.3-1 micron, about 0.4-1 micron, about 0.5-1 micron, about 0.6-1 micron, about 0.7-1 micron, about 0.8-1 micron or about 0.9-1 micron.
173. The method of any one of paragraphs 158-159 or 162-163 wherein the depth filter has a nominal retention range of between about between about 0.05-50 micron, 0.1-25 micron 0.2-10, micron 0.1-10 micron, 0.2-5 micron or 0.25-1 micron.
174. The method of any one of paragraphs 158-159 or 162-173 wherein the depth filter has a filter capacity of 1-2500 L/m², 5-2500 L/m², 10-2500 L/m², 25-2500 L/m², 50-2500 L/m², 75-2500 L/m², 100-2500 L/m², 150-2500 L/m², 200-2500 L/m², 300-2500 L/m², 400-2500 L/m², 500-2500 L/m², 750-2500 L/m², 1000-2500 L/m², 1500-2500 L/m² or 2000-2500 L/m².
175. The method of any one of paragraphs 158-159 or 162-173 wherein the depth filter has a filter capacity of 1-1000 L/m², 5-1000 L/m², 10-1000 L/m², 25-1000 L/m², 50-1000 L/m², 75-1000 L/m², 100-1000 L/m², 150-1000 L/m², 200-1000 L/m², 300-1000 L/m², 400-1000 L/m², 500-1000 L/m² or 750-1000 L/m².
176. The method of any one of paragraphs 155-156 or 159-170 wherein the depth filter has a filter capacity of 1-750 L/m², 5-750 L/m², 10-750 L/m², 25-750 L/m², 50-750 L/m², 75-750 L/m², 100-750 L/m², 150-750 L/m², 200-750 L/m², 300-750 L/m², 400-750 L/m² or 500-750 L/m².
177. The method of any one of paragraphs 158-159 or 162-173 wherein the depth filter has a filter capacity of 1-500 L/m², 5-500 L/m², 10-500 L/m², 25-500 L/m², 50-500 L/m², 75-500 L/m², 100-500 L/m², 150-500 L/m², 200-500 L/m², 300-500 L/m² or 400-500 L/m².
178. The method of any one of paragraphs 158-159 or 162-173 wherein the depth filter has a filter capacity of 1-400 L/m², 5-400 L/m², 10-400 L/m², 25-400 L/m², 50-400 L/m², 75-400 L/m², 100-400 L/m², 150-400 L/m², 200-400 L/m² or 300-400 L/m².
179. The method of any one of paragraphs 158-159 or 162-173 wherein the depth filter has a filter capacity of 1-300 L/m², 5-300 L/m², 10-300 L/m², 25-300 L/m², 50-300 L/m², 75-300 L/m², 100-300 L/m², 150-300 L/m² or 200-300 L/m².
180. The method of any one of paragraphs 158-159 or 162-173 wherein the depth filter has a filter capacity of 1-200 L/m², 5-200 L/m², 10-200 L/m², 25-200 L/m², 50-200 L/m², 75-200 L/m², 100-200 L/m² or 150-200 L/m².
181. The method of any one of paragraphs 158-159 or 162-173 wherein the depth filter has a filter capacity of 1-100 L/m², 5-100 L/m², 10-100 L/m², 25-100 L/m², 50-100 L/m² or 75-100 L/m².
182. The method of any one of paragraphs 158-159 or 162-173 wherein the depth filter has a filter capacity of 1-50 L/m², 5-50 L/m², 10-50 L/m² or 25-50 L/m².
183. The method of any one of paragraphs 158-159 or 162-182 wherein the feed rate is between 1-1000 LMH (liters/m²/hour), 10-1000 LMH, 25-1000 LMH, 50-1000 LMH, 100-1000 LMH, 125-1000 LMH, 150-1000 LMH, 200-1000 LMH, 250-1000 LMH, 300-1000 LMH, 400-1000 LMH, 500-1000 LMH, 600-1000 LMH, 700-1000 LMH, 800-1000 LMH or 900-1000 LMH.
184. The method of any one of paragraphs 158-159 or 162-182 wherein the feed rate is between 1-500 LMH, 10-500 LMH, 25-500 LMH, 50-500 LMH, 100-500 LMH, 125-500 LMH, 150-500 LMH, 200-500 LMH, 250-500 LMH, 300-500 LMH or 400-500 LMH.
185. The method of any one of paragraphs 158-159 or 162-182 wherein the feed rate is between 1-400 LMH, 10-400 LMH, 25-400 LMH, 50-400 LMH, 100-400 LMH, 125-400 LMH, 150-400 LMH, 200-400 LMH, 250-400 LMH or 300-400 LMH.
186. The method of any one of paragraphs 158-159 or 162-182 wherein the feed rate is between 1-250 LMH, 10-250 LMH, 25-250 LMH, 50-250 LMH, 100-250 LMH, 125-250 LMH, 150-250 LMH or 200-250 LMH.
187. The method of any one of paragraphs 158-159 or 162-182 wherein the feed rate is about 1, about 2, about 5, about 10, about 25, about 50, about 60, about 70, about 80, about 90, about 100, about 110, about 120, about 130, about 140, about 150, about 160, about 170, about 180, about 190, about 200, about 210, about 220, about 230, about 240 about 250, about 260, about 270, about 280, about 290, about 300, about 310, about 320, about 330, about 340, about 350, about 360, about 370, about 380, about 390, about 400, about 425, about 450, about 475, about 500, about 525, about 550, about 575, about 600, about 650, about 700, about 750, about 800, about 850, about 900, about 950 or about 1000 LMH.
188. The method of any one of paragraphs 158-187 wherein the filtrate is subjected to microfiltration.
189. The method of paragraph 188 wherein the said microfiltration is dead-end filtration.
190. The method of paragraph 188 wherein the said microfiltration is tangential microfiltration.
191. The method of any one of paragraphs 188-190 wherein the microfiltration filter has a nominal retention range of between about 0.01-2 micron, about 0.05-2 micron, about 0.1-2 micron, about 0.2-2 micron, about 0.3-2 micron, about 0.4-2 micron, about 0.45-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, or about 1.75-2 micron.
192. The method of any one of paragraphs 188-190 wherein the microfiltration filter has a nominal retention range of between about 0.01-1 micron, about 0.05-1 micron, about 0.1-1 micron, about 0.2-1 micron, about 0.3-1 micron, about 0.4-1 micron, about 0.45-1 micron, about 0.5-1 micron, about 0.6-1 micron, about 0.7-1 micron, about 0.8-1 micron or about 0.9-1 micron.
193. The method of any one of paragraphs 188-190 wherein the microfiltration filter has a nominal retention range of about 0.01, about 0.05, about 0.1, about 0.2, about 0.3, about 0.4, about 0.45, about 0.5, about 0.6, about 0.7, about 0.8, about 0.9, about 1, about 1.1, about 1.2, about 1.3, about 1.4, about 1.5, about 1.6, about 1.7, about 1.8, about 1.9 or about 2 micron.
194. The method of any one of paragraphs 188-190 wherein the microfiltration filter has a nominal retention of about 0.45 micron.
195. The method of any one of paragraphs 188-194 wherein the microfiltration filter has a filter capacity of between 100-5000 L/m², 200-5000 L/m², 300-5000 L/m², 400-5000 L/m², 500-5000 L/m², 750-5000 L/m², 1000-5000 L/m², 1500-5000 L/m², 2000-5000 L/m², 3000-5000 L/m² or 4000-5000 L/m².
196. The method of any one of paragraphs 188-194 wherein the microfiltration filter has a filter capacity of between 100-2500 L/m², 200-2500 L/m², 300-2500 L/m², 400-2500 L/m², 500-2500 L/m², 750-2500 L/m², 1000-2500 L/m², 1500-2500 L/m² or 2000-2500 L/m².
197. The method of any one of paragraphs 188-194 wherein the microfiltration filter has a filter capacity of between 100-1500 L/m², 200-1500 L/m², 300-1500 L/m², 400-1500 L/m², 500-1500 L/m², 750-1500 L/m² or 1000-1500 L/m².
198. The method of any one of paragraphs 188-194 wherein the microfiltration filter has a filter capacity of between 100-1250 L/m², 200-1250 L/m², 300-1250 L/m², 400-1250 L/m², 500-1250 L/m², 750-1250 L/m² or 1000-1250 L/m².
199. The method of any one of paragraphs 188-194 wherein the microfiltration filter has a filter capacity of between 100-1000 L/m², 200-1000 L/m², 300-1000 L/m², 400-1000 L/m², 500-1000 L/m² or 750-1000 L/m².
200. The method of any one of paragraphs 188-194 wherein the microfiltration filter has a filter capacity of between 100-750 L/m², 200-750 L/m², 300-750 L/m², 400-750 L/m² or 500-750 L/m².
201. The method of any one of paragraphs 188-194 wherein the microfiltration filter has a filter capacity of between 100-600 L/m², 200-600 L/m², 300-600 L/m², 400-600 L/m² or 400-600 L/m².
202. The method of any one of paragraphs 188-194 wherein the microfiltration filter has a filter capacity of between 100-500 L/m², 200-500 L/m², 300-500 L/m² or 400-500 L/m².
203. The method of any one of paragraphs 188-194 wherein the microfiltration filter has a filter capacity of 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450 or about 2500 L/m².
204. The method of any one of paragraphs 158-203 wherein the filtrate is further treated by Ultrafiltration and/or Dialfiltration.
205. The method of any one of paragraphs 158-203 wherein the filtrate is further treated by ultrafiltration.
206. The method of any one of paragraphs 204-205 wherein the molecular weight cut off of the ultrafiltration membrane is in the range of between about 5 kDa-1000 kDa.
207. The method of any one of paragraphs 204-205 wherein the molecular weight cut off of the ultrafiltration membrane is in the range of between about 10 kDa-750 kDa.
208. The method of any one of paragraphs 204-205 wherein the molecular weight cut off of the ultrafiltration membrane is in the range of between about 10 kDa-500 kDa.
209. The method of any one of paragraphs 204-205 wherein the molecular weight cut off of the ultrafiltration membrane is in the range of between about 10 kDa-300 kDa.
210. The method of any one of paragraphs 204-205 wherein the molecular weight cut off of the ultrafiltration membrane is in the range of between about 10 kDa-100 kDa.
211. The method of any one of paragraphs 204-205 wherein the molecular weight cut off of the ultrafiltration membrane is in the range of between about 10 kDa-50 kDa.
212. The method of any one of paragraphs 204-205 wherein the molecular weight cut off of the ultrafiltration membrane is in the range of between about 10 kDa-30 kDa.
213. The method of any one of paragraphs 204-205 wherein the molecular weight cut off of the ultrafiltration membrane is in the range of between about about 5 kDa-1000 kDa, about 10 kDa-1000 kDa about 20 kDa-1000 kDa, about 30 kDa-1000 kDa, about 40 kDa-1000 kDa, about 50 kDa-1000 kDa, about 75 kDa-1000 kDa, about 100 kDa-1000 kDa, about 150 kDa-1000 kDa, about 200 kDa-1000 kDa, about 300 kDa-1000 kDa, about 400 kDa-1000 kDa, about 500 kDa-1000 kDa or about 750 kDa-1000 kDa.
214. The method of any one of paragraphs 204-205 wherein the molecular weight cut off of the ultrafiltration membrane is in the range of between about 5 kDa-500 kDa, about 10 kDa-500 kDa, about 20 kDa-500 kDa, about 30 kDa-500 kDa, about 40 kDa-500 kDa, about 50 kDa-500 kDa, about 75 kDa-500 kDa, about 100 kDa-500 kDa, about 150 kDa-500 kDa, about 200 kDa-500 kDa, about 300 kDa-500 kDa or about 400 kDa-500 kDa.
215. The method of any one of paragraphs 204-205 wherein the molecular 5 kDa-300 kDa, about 10 kDa-300 kDa, about 20 kDa-300 kDa, about 30 kDa-300 kDa, about 40 kDa-300 kDa, about 50 kDa-300 kDa, about 75 kDa-300 kDa, about 100 kDa-300 kDa, about 150 kDa-300 kDa or about 200 kDa-300 kDa.
216. The method of any one of paragraphs 204-205 wherein the molecular weight cut off of the ultrafiltration membrane is in the range of between about 5 kDa-100 kDa, about 10 kDa-100 kDa, about 20 kDa-100 kDa, about 30 kDa-100 kDa, about 40 kDa-100 kDa, about 50 kDa-100 kDa or about 75 kDa-100 kDa.
217. The method of any one of paragraphs 204-205 wherein the molecular weight cut off of the ultrafiltration membrane is about 5 kDa, about 10 kDa, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 200 kDa, about 250 kDa, about 300 kDa, about 400 kDa, about 500 kDa, about 750 kDa or about 1000 kDa.
218. The method of any one of paragraphs 204-217 wherein the concentration factor of the ultrafiltration step is from about 1.5 to about 10.
219. The method of any one of paragraphs 204-217 wherein the concentration factor is from about 2 to about 8.
220. The method of any one of paragraphs 204-217 wherein the concentration factor is from about 2 to about 5.
221. The method of any one of paragraphs 204-217 wherein the concentration factor is about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0.
222. The method of any one of paragraphs 204-217 wherein the concentration factor is about 2, about 3, about 4, about 5, or about 6.
223. The method of any one of paragraphs 204-222 wherein said ultrafiltration step is performed at temperature between about 20° C. to about 90° C.
224. The method of any one of paragraphs 204-222 wherein said ultrafiltration step is performed at temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
225. The method of any one of paragraphs 204-222 wherein said ultrafiltration step is performed at temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
226. The method of any one of paragraphs 204-222 wherein said ultrafiltration step is performed at temperature of about 50° C.
227. The method of any one of paragraphs 158-226 wherein the ultrafiltration filtrate is treated by diafiltration.
228. The method of paragraph 227 wherein the replacement solution is water.
229. The method of paragraph 227 wherein the replacement solution is saline in water.
230. The method of paragraph 229 wherein the salt is selected from the group consisting of magnesium chloride, potassium chloride, sodium chloride and a combination thereof.
231. The method of paragraph 229 wherein the salt is sodium chloride.
232. The method of paragraph 229 wherein the replacement solution is sodium chloride at about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM or about 500 mM.
233. The method of paragraph 227 wherein the replacement solution is a buffer solution.
234. The method of paragraph 227 wherein the replacement solution is a buffer solution wherein the buffer is selected from the group consisting of N-(2-Acetamido)-aminoethanesulfonic acid (ACES), a salt of acetic acid (acetate), N-(2-Acetamido)-iminodiacetic acid (ADA), 2-Aminoethanesulfonic acid (AES, Taurine), ammonia, 2-Amino-2-methyl-1-propanol (AMP), 2-Amino-2-methyl-1,3-propanediol AMPD, ammediol, N-(1,1-Dimethyl-2-hydroxyethyl)-3-amino-2-hydroxypropanesulfonic acid (AMPSO), N, N-Bis-(2-hydroxyethyl)-2-aminoethanesulfonic acid (BES), sodium hydrogen carbonate (bicarbonate), N, N′-Bis(2-hydroxyethyl)-glycine (bicine), [Bis-(2-hydroxyethyl)-imino]-tris-(hydroxymethylmethane) (BIS-Tris), 1,3-Bis[tris(hydroxymethyl)-methylamino]propane (BIS-Tris-Propane), Boric acid, dimethylarsinic acid (Cacodylate), 3-(Cyclohexylamino)-propanesulfonic acid (CAPS), 3-(Cyclohexylamino)-2-hydroxy-1-propanesulfonic acid (CAPSO), sodium carbonate (Carbonate), cyclohexylaminoethanesulfonic acid (CHES), a salt of citric acid (citrate), 3[N-Bis(hydroxyethyl)amino]-2-hydroxypropanesulfonic acid (DIPSO), a salt of formic acid (formate), Glycine, Glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, a salt of malic acid (Malate), a salt of maleic acid (Maleate), 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), a salt of phosphoric acid (Phosphate), Piperazine-N,N′-bis(2-ethanesulfonic acid) (PIPES), Piperazine-N,N′-bis(2-hydroxypropanesulfonic acid) (POPSO), pyridine, a salt of succinic acid (Succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine) and Tris(hydroxymethyl)-aminomethane (Tris).
235. The method of paragraph 227 wherein the replacement solution is a buffer solution wherein the buffer is selected from the group consisting of a salt of acetic acid (acetate), a salt of citric acid (citrate), a salt of formic acid (formate), a salt of malic acid (Malate), a salt of maleic acid (Maleate), a salt of phosphoric acid (Phosphate) and a salt of succinic acid (Succinate).
236. The method of paragraph 227 wherein the replacement solution is a buffer solution wherein the buffer is a salt of citric acid (citrate).
237. The method of paragraph 227 wherein the replacement solution is a buffer solution wherein the buffer is a salt of succinic acid (Succinate).
238. The method of any one of paragraphs 234-237 said salt is a sodium salt.
239. The method of any one of paragraphs 234-237 said salt is a potassium salt.
240. The method of any one of paragraphs 233-239 wherein the pH of the diafiltration buffer is between about 4.0-11.0, between about 5.0-10.0, between about 5.5-9.0, between about 6.0-8.0, between about 6.0-7.0, between about 6.5-7.5, between about 6.5-7.0 or between about 6.0-7.5.
241. The method of any one of paragraphs 233-239 wherein the pH of the diafiltration buffer is about 4.0, about 4.5, about 5.0, about 5.5, about 6.0, about 6.5, about 7.0, about 7.5, about 8.0, about 8.5, about 9.0, about 9.5, about 10.0, about 10.5 or about 11.0.
242. The method of any one of paragraphs 233-239 wherein the pH of the diafiltration buffer is about 6.0, about 6.5, about 7.0, about 7.5, about 8.0, about 8.5 or about 9.0.
243. The method of any one of paragraphs 226-231 wherein the pH of the diafiltration buffer is about 6.5, about 7.0 or about 7.5.
244. The method of any one of paragraphs 233-239 wherein the pH of the diafiltration buffer is about 7.0.
245. The method of any one of paragraphs 233-244 wherein the concentration of the diafiltration buffer is between about 0.01 mM-100 mM, between about 0.1 mM-100 mM, between about 0.5 mM-100 mM, between about 1 mM-100 mM, between about 2 mM-100 mM, between about 3 mM-100 mM, between about 4 mM-100 mM, between about 5 mM-100 mM, between about 6 mM-100 mM, between about 7 mM-100 mM, between about 8 mM-100 mM, between about 9 mM-100 mM, between about 10 mM-100 mM, between about 11 mM-100 mM, between about 12 mM-100 mM, between about 13 mM-100 mM, between about 14 mM-100 mM, between about 15 mM-100 mM, between about 16 mM-100 mM, between about 17 mM-100 mM, between about 18 mM-100 mM, between about 19 mM-100 mM, between about 20 mM-100 mM, between about 25 mM-100 mM, between about 30 mM-100 mM, between about 35 mM-100 mM, between about 40 mM-100 mM, between about 45 mM-100 mM, between about 50 mM-100 mM, between about 55 mM-100 mM, between about 60 mM-100 mM, between about 65 mM-100 mM, between about 70 mM-100 mM, between about 75 mM-100 mM, between about 80 mM-100 mM, between about 85 mM-100 mM, between about 90 mM-100 mM or between about 95 mM-100 mM.
246. The method of any one of paragraphs 233-244 wherein the concentration of the diafiltration buffer is between about 0.01 mM-50 mM, between about 0.1 mM-50 mM, between about 0.5 mM-50 mM, between about 1 mM-50 mM, between about 2 mM-50 mM, between about 3 mM-50 mM, between about 4 mM-50 mM, between about 5 mM-50 mM, between about 6 mM-50 mM, between about 7 mM-50 mM, between about 8 mM-50 mM, between about 9 mM-50 mM, between about 10 mM-50 mM, between about 11 mM-50 mM, between about 12 mM-50 mM, between about 13 mM-50 mM, between about 14 mM-50 mM, between about 15 mM-50 mM, between about 16 mM-50 mM, between about 17 mM-50 mM, between about 18 mM-50 mM, between about 19 mM-50 mM, between about 20 mM-50 mM, between about 25 mM-50 mM, between about 30 mM-50 mM, between about 35 mM-50 mM, between about 40 mM-50 mM or between about 45 mM-50 mM.
247. The method of any one of paragraphs 233-244 wherein the concentration of the diafiltration buffer is between about 0.01 mM-25 mM, between about 0.1 mM-25 mM, between about 0.5 mM-25 mM, between about 1 mM-25 mM, between about 2 mM-25 mM, between about 3 mM-25 mM, between about 4 mM-25 mM, between about 5 mM-25 mM, between about 6 mM-25 mM, between about 7 mM-25 mM, between about 8 mM-25 mM, between about 9 mM-25 mM, between about 10 mM-25 mM, between about 11 mM-25 mM, between about 12 mM-25 mM, between about 13 mM-25 mM, between about 14 mM-25 mM, between about 15 mM-25 mM, between about 16 mM-25 mM, between about 17 mM-25 mM, between about 18 mM-25 mM, between about 19 mM-25 mM or between about 20 mM-25 mM.
248. The method of any one of paragraphs 233-244 wherein the concentration of the diafiltration buffer is between about 0.01 mM-15 mM, between about 0.1 mM-15 mM, between about 0.5 mM-15 mM, between about 1 mM-15 mM, between about 2 mM-15 mM, between about 3 mM-15 mM, between about 4 mM-15 mM, between about 5 mM-15 mM, between about 6 mM-15 mM, between about 7 mM-15 mM, between about 8 mM-15 mM, between about 9 mM-15 mM, between about 10 mM-15 mM, between about 11 mM-15 mM, between about 12 mM-15 mM, between about 13 mM-15 mM or between about 14 mM-15 mM.
249. The method of any one of paragraphs 233-244 wherein the concentration of the diafiltration buffer is between about 0.01 mM-10 mM, between about 0.1 mM-10 mM, between about 0.5 mM-10 mM, between about 1 mM-10 mM, between about 2 mM-10 mM, between about 3 mM-10 mM, between about 4 mM-10 mM, between about 5 mM-10 mM, between about 6 mM-10 mM, between about 7 mM-10 mM, between about 8 mM-10 mM or between about 9 mM-10 mM.
250. The method of any one of paragraphs 233-244 wherein the concentration of the diafiltration buffer is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.
251. The method of any one of paragraphs 233-244 wherein the concentration of the diafiltration buffer is about 0.1 mM, about 0.2 mM, about 1 mM, about 5 mM, about 10 mM, about 15 mM, about 20 mM, about 30 mM, about 40 mM, or about 50 mM.
252. The method of any one of paragraphs 233-244 wherein the concentration of the diafiltration buffer is about 10 mM.
253. The method of any one of paragraphs 233-252 wherein the replacement solution comprises a chelating agent.
254. The method of any one of paragraphs 233-252 wherein the replacement solution comprises an alum chelating agent.
255. The method of any one of paragraphs 233-252 wherein the replacement solution comprises a chelating agent selected from the groups consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N, N,N′,N′-tetraacetic acid (DPTA-OH), ethylenediamine-N, N′-bis(2-hydroxyphenylacetic acid) (EDDHA), ethylenediamine-N, N′-dipropionic acid dihydrochloride (EDDP), ethylenediamine-tetrakis(methylenesulfonic acid) (EDTPO), Nitrilotris(methylenephosphonic acid) (NTPO), imino-diacetic acid (IDA), hydroxyimino-diacetic acid (HIDA), nitrilo-triacetic acid (NTP), triethylenetetramine-hexaacetic acid (TTHA), Dimercaptosuccinic acid (DMSA), 2,3-dimercapto-1-propanesulfonic acid (DMPS), alpha lipoic acid (ALA), Nitrilotriacetic acid (NTA), thiamine tetrahydrofurfuryl disulfide (TTFD), dimercaprol, penicillamine, deferoxamine (DFOA), deferasirox, phosphonates, a salt of citric acid (citrate) and combinations of these.
256. The method of any one of paragraphs 233-255 wherein the replacement solution comprises a chelating agent selected from the groups consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N, N, N′,N¹-tetraacetic acid (EGTA), 1,2-cyclohexanediamine-N,N,N′,N′-tetraacetic acid (CyDTA), diethylenetriamine-N, N, N′,N″,N″-pentaacetic acid (DTPA), 1,3-diaminopropan-2-ol-N,N,N′,N′-tetraacetic acid (DPTA-OH), ethylenediamine-N, N′-bis(2-hydroxyphenylacetic acid) (EDDHA), a salt of citric acid (citrate) and combinations of these.
257. The method of any one of paragraphs 233-254 wherein the replacement solution comprises Ethylene Diamine Tetra Acetate (EDTA) as chelating agent.
258. The method of any one of paragraphs 233-254 wherein the replacement solution comprises a salt of citric acid (citrate) as chelating agent.
259. The method of any one of paragraphs 233-254 wherein the replacement solution comprises sodium citrate as chelating agent.
260. The method of any one of paragraphs 253-258 wherein the concentration of the chelating agent in the replacement solution is from 1 to 500 mM.
261. The method of any one of paragraphs 253-258 wherein the concentration of the chelating agent in the replacement solution is from 2 to 400 mM.
262. The method of any one of paragraphs 253-258 wherein concentration of the chelating agent in the replacement solution is from 10 to 400 mM.
263. The method of any one of paragraphs 253-258 wherein concentration of the chelating agent in the replacement solution is from 10 to 200 mM.
264. The method of any one of paragraphs 253-258 wherein concentration of the chelating agent in the replacement solution is from 10 to 100 mM.
265. The method of any one of paragraphs 253-258 wherein concentration of the chelating agent in the replacement solution is from 10 to 50 mM.
266. The method of any one of paragraphs 253-258 wherein concentration of the chelating agent in the replacement solution is from 10 to 30 mM.
267. The method of any one of paragraphs 253-258 wherein concentration of the chelating agent in the replacement solution is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.
268. The method of any one of paragraphs 253-258 wherein concentration of the chelating agent in the replacement solution is about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM or about 100 mM.
269. The method of any one of paragraphs 253-258 wherein concentration of the chelating agent in the replacement solution is about 15 mM, about 20 mM, about 25 mM, about 30 mM, about 35 mM, about 40 mM, about 45 mM or about 50 mM.
270. The method of any one of paragraphs 233-269 wherein the replacement solution comprises a salt.
271. The method of paragraph 270 wherein, the salt is selected from the groups consisting of magnesium chloride, potassium chloride, sodium chloride and a combination thereof.
272. The method of paragraph 270 wherein, the salt is sodium chloride.
273. The method of any one of paragraphs 270-272 wherein the replacement solution comprises sodium chloride at 1 about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250 or about 300 mM.
274. The method of any one of paragraphs 227-273 wherein the number of diavolumes is at least 5, 10, 15, 20, 25, 30, 35, 40, 45, or 50.
275. The method of any one of paragraphs 227-273 wherein the number of diavolumes is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95 or about 100.
276. The method of any one of paragraphs 227-273 wherein the number of diavolumes is about 5, about 6, about 7, about 8, about 9, about 10, about 11, about 12, about 13, about 14 or about 15.
277. The method of any one of paragraphs 227-276 wherein said dialfiltration step is performed at temperature of between about 20° C. to about 90° C.
278. The method of any one of paragraphs 227-276 wherein said dialfiltration step is performed at temperature of between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
279. The method of any one of paragraphs 227-276 wherein said dialfiltration step is performed at temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
280. The method of any one of paragraphs 227-276 wherein said dialfiltration step is performed at temperature of about 50° C.
281. The method of any one of paragraphs 204-277 wherein said ultrafiltration and dialfiltration steps if both conducted are performed at a temperature between about 20° C. to about 90° C.
282. The method of any one of paragraphs 204-277 wherein said ultrafiltration and dialfiltration steps if both conducted are performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
283. The method of any one of paragraphs 204-277 wherein said ultrafiltration and dialfiltration steps if both conducted are performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
284. The method of any one of paragraphs 204-277 wherein said ultrafiltration and dialfiltration steps if both conducted are performed at a temperature of about 50° C.
285. The method of any one of paragraphs 1-284 wherein the solution containing the polysaccharide (e.g. the supernatant, the filtrate or retentate) is treated by an activated carbon filtration step.
286. The method of any one of paragraph 285 wherein the activated carbon is added in the form of a powder, as a granular carbon bed, as a pressed carbon block or extruded carbon block (see e.g. Norit active charcoal).
287. The method of paragraph 286 wherein the activated carbon is added in an amount of about 0.1 to 20% (weight volume), 1 to 15% (weight volume), 1 to 10% (weight volume), 2 to 10% (weight volume), 3 to 10% (weight volume), 4 to 10% (weight volume), 5 to 10% (weight volume), 1 to 5% (weight volume) or 2 to 5% (weight volume).
288. The method of any one of paragraph 286-287 wherein the mixture is stirred and left to stand.
289. The method of any one of paragraph 286-287 wherein the mixture is stirred and left to stand for about 5, about 10, about 15, about 20, about 30, about 45, about 60, about 90, about 120, about 180, about 240 minutes or more.
290. The method of any one of paragraph 286-289 wherein the activated carbon is then removed.
291. The method of any one of paragraph 286-290 wherein the activated carbon is removed by centrifugation or filtration.
292. The method of paragraph 285 wherein the solution is filtered through activated carbon immobilized in a matrix.
293. The method of paragraph 285 wherein said matrix is a porous filter medium permeable for the solution.
294. The method of any one of paragraphs 292-293 wherein said matrix comprises a support material.
295. The method of any one of paragraphs 292-293 wherein said matrix comprises a binder material.
296. The method of any one of paragraphs 294-295 wherein said support material is a synthetic polymer.
297. The method of any one of paragraphs 294-295 wherein said support material is a polymer of natural origin.
298. The method of paragraph 296 wherein said synthetic polymers includes any one of polystyrene, polyacrylamide or polymethyl methacrylate.
299. The method of paragraph 296 wherein said synthetic polymers is selected from the group consisting of polystyrene, polyacrylamide and polymethyl methacrylate.
300. The method of paragraph 297 wherein said a polymer of natural origin include includes any one of cellulose, polysaccharide, dextran or agarose.
301. The method of paragraph 297 wherein said a polymer of natural is selected from the group consisting of cellulose, polysaccharide, dextran and agarose.
302. The method of any one of paragraphs 294-301 wherein said polymer support material if present is in the form of a fibre network to provide mechanical rigidity.
303. The method of any one of paragraphs 294-302 wherein said binder material if present is a resin.
304. The method of any one of paragraphs 292-303 wherein said matrix has the form of a membrane sheet.
305. The method of any one of paragraphs 292-304 wherein the activated carbon immobilized in the matrix is in the form of a flow-through carbon cartridge.
306. The method of any one of paragraphs 304 wherein the membrane sheet is spirally wound.
307. The method of any one of paragraphs 292-306, wherein several discs are stacked upon each other.
308. The method of paragraphs 307 wherein the configuration of stacked discs is lenticular.
309. The method of any one of paragraphs 292-308, wherein the activated carbon in the carbon filter is derived from peat, lignite, wood or coconut shell.
310. The method of any one of paragraphs 292-309, wherein the activated carbon immobilized in a matrix is placed in a housing to form an independent filter unit.
311. The method of any one of paragraphs 292-310, wherein the activated carbon filters comprise a cellulose matrix into which activated carbon powder is entrapped and resin-bonded in place.
312. The method of any one of paragraphs 285-311, wherein the activated carbon filter has a nominal micron rating of between about 0.01-100 micron, about 0.05-100 micron, about 0.1-100 micron, about 0.2-100 micron, about 0.3-100 micron, about 0.4-100 micron, about 0.5-100 micron, about 0.6-100 micron, about 0.7-100 micron, about 0.8-100 micron, about 0.9-100 micron, about 1-100 micron, about 1.25-100 micron, about 1.5-100 micron, about 1.75-100 micron, about 2-100 micron, about 3-100 micron, about 4-100 micron, about 5-100 micron, about 6-100 micron, about 7-100 micron, about 8-100 micron, about 9-100 micron, about 10-100 micron, about 15-100 micron, about 20-100 micron, about 25-100 micron, about 30-100 micron, about 40-100 micron, about 50-100 micron or about 75-100 micron.
313. The method of any one of paragraphs 285-311, wherein the activated carbon filter has a nominal micron rating of between about 0.01-50 micron, about 0.05-50 micron, about 0.1-50 micron, about 0.2-50 micron, about 0.3-50 micron, about 0.4-50 micron, about 0.5-50 micron, about 0.6-50 micron, about 0.7-50 micron, about 0.8-50 micron, about 0.9-50 micron, about 1-50 micron, about 1.25-50 micron, about 1.5-50 micron, about 1.75-50 micron, about 2-50 micron, about 3-50 micron, about 4-50 micron, about 5-50 micron, about 6-50 micron, about 7-50 micron, about 8-50 micron, about 9-50 micron, about 10-50 micron, about 15-50 micron, about 20-50 micron, about 25-50 micron, about 30-50 micron, about 40-50 micron or about 50-50 micron.
314. The method of any one of paragraphs 285-311, wherein the activated carbon filter has a nominal micron rating of between about 0.01-25 micron, about 0.05-25 micron, about 0.1-25 micron, about 0.2-25 micron, about 0.3-25 micron, about 0.4-25 micron, about 0.5-25 micron, about 0.6-25 micron, about 0.7-25 micron, about 0.8-25 micron, about 0.9-25 micron, about 1-25 micron, about 1.25-25 micron, about 1.5-25 micron, about 1.75-25 micron, about 2-25 micron, about 3-25 micron, about 4-25 micron, about 5-25 micron, about 6-25 micron, about 7-25 micron, about 8-25 micron, about 9-25 micron, about 10-25 micron, about 15-25 micron or about 20-25 micron.
315. The method of any one of paragraphs 285-311, wherein the activated carbon filter has a nominal micron rating of between about 0.01-10 micron, about 0.05-10 micron, about 0.1-10 micron, about 0.2-10 micron, about 0.3-10 micron, about 0.4-10 micron, about 0.5-10 micron, about 0.6-10 micron, about 0.7-10 micron, about 0.8-10 micron, about 0.9-10 micron, about 1-10 micron, about 1.25-10 micron, about 1.5-10 micron, about 1.75-10 micron, about 2-10 micron, about 3-10 micron, about 4-10 micron, about 5-10 micron, about 6-10 micron, about 7-10 micron, about 8-10 micron or about 9-10 micron.
316. The method of any one of paragraphs 285-311, wherein the activated carbon filter has a nominal micron rating of between about 0.01-8 micron, about 0.05-8 micron, about 0.1-8 micron, about 0.2-8 micron, about 0.3-8 micron, about 0.4-8 micron, about 0.5-8 micron, about 0.6-8 micron, about 0.7-8 micron, about 0.8-8 micron, about 0.9-8 micron, about 1-8 micron, about 1.25-8 micron, about 1.5-8 micron, about 1.75-8 micron, about 2-8 micron, about 3-8 micron, about 4-8 micron, about 5-8 micron, about 6-8 micron or about 7-8 micron.
317. The method of any one of paragraphs 285-311, wherein the activated carbon filter has a nominal micron rating of between about 0.01-5 micron, about 0.05-5 micron, about 0.1-5 micron, about 0.2-5 micron, about 0.3-5 micron, about 0.4-5 micron, about 0.5-5 micron, about 0.6-5 micron, about 0.7-5 micron, about 0.8-5 micron, about 0.9-5 micron, about 1-5 micron, about 1.25-5 micron, about 1.5-5 micron, about 1.75-5 micron, about 2-5 micron, about 3-5 micron or about 4-5 micron.
318. The method of any one of paragraphs 285-311, wherein the activated carbon filter has a nominal micron rating of between about 0.01-2 micron, about 0.05-2 micron, about 0.1-2 micron, about 0.2-2 micron, about 0.3-2 micron, about 0.4-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, about 1.75-2 micron, about 2-2 micron, about 3-2 micron or about 4-2 micron.
319. The method of any one of paragraphs 285-311, wherein the activated carbon filter has a nominal micron rating of between about 0.01-1 micron, about 0.05-1 micron, about 0.1-1 micron, about 0.2-1 micron, about 0.3-1 micron, about 0.4-1 micron, about 0.5-1 micron, about 0.6-1 micron, about 0.7-1 micron, about 0.8-1 micron or about 0.9-1 micron.
320. The method of any one of paragraphs 285-311, wherein the activated carbon filter has a nominal micron rating of between about 0.05-50 micron, 0.1-25 micron 0.2-10, micron 0.1-10 micron, 0.2-5 micron or 0.25-1 micron.
321. The method of any one of paragraphs 285-320, wherein the activated carbon filter is conducted at a feed rate of between 1-500 LMH, 10-500 LMH, 15-500 LMH, 20-500 LMH, 25-500 LMH, 30-500 LMH, 40-500 LMH, 50-500 LMH, 100-500 LMH, 125-500 LMH, 150-500 LMH, 200-500 LMH, 250-500 LMH, 300-500 LMH or 400-500 LMH.
322. The method of any one of paragraphs 285-320, wherein the activated carbon filter is conducted at a feed rate of between 1-200 LMH, 10-200 LMH, 15-200 LMH, 20-200 LMH, 25-200 LMH, 30-200 LMH, 40-200 LMH, 50-200

LMH, 100-200 LMH, 125-200 LMH or 150-200 LMH.
323. The method of any one of paragraphs 285-320, wherein the activated carbon filter is conducted at a feed rate of between 1-150 LMH, 10-150 LMH, 15-150 LMH, 20-150 LMH, 25-150 LMH, 30-150 LMH, 40-150 LMH, 50-150 LMH, 100-150 LMH or 125-150 LMH.
324. The method of any one of paragraphs 285-320, wherein the activated carbon filter is conducted at a feed rate of between 1-100 LMH, 10-100 LMH, 15-100 LMH, 20-100 LMH, 25-100 LMH, 30-100 LMH, 40-100 LMH, or 50-100 LMH.
325. The method of any one of paragraphs 285-320, wherein the activated carbon filter is conducted at a feed rate of between 1-75 LMH, 5-75 LMH, 10-75 LMH, 15-75 LMH, 20-75 LMH, 25-75 LMH, 30-75 LMH, 35-75 LMH, 40-75 LMH, 45-75 LMH, 50-75 LMH, 55-75 LMH, 60-75 LMH, 65-75 LMH, or 70-75 LMH.
326. The method of any one of paragraphs 285-320, wherein the activated carbon filter is conducted at a feed rate of between 1-50 LMH, 5-50 LMH, 7-50 LMH, 10-50 LMH, 15-50 LMH, 20-50 LMH, 25-50 LMH, 30-50 LMH, 35-50 LMH, 40-50 LMH or 45-50 LMH.
327. The method of any one of paragraphs 285-320, wherein the activated carbon filter is conducted at a feed rate of about 1, about 2, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 300, about 350, about 400, about 450, about 500, about 550, about 600, about 700, about 800, about 900, about 950 or about 1000 LMH.
328. The method of any one of paragraphs 285-327, wherein the solution is treated by an activated carbon filter wherein the filter has a filter capacity of between 5-1000 L/m², 10-750 L/m², 15-500 L/m², 20-400 L/m², 25-300 L/m², 30-250 L/m², 40-200 L/m² or 30-100 L/m².
329. The method of any one of paragraphs 285-327, wherein the solution is treated by an activated carbon filter wherein the filter has a filter capacity of about 5, about 10, about 15, about 20, about 25, about 30, about 35, about 40, about 45, about 50, about 55, about 60, about 65, about 70, about 75, about 80, about 85, about 90, about 100, about 125, about 150, about 175, about 200, about 225, about 250, about 275, about 300, about 400, about 500, about 600, about 700, about 800, about 900, or about 1000 L/m².
330. The method of any one of paragraphs 285-329, wherein 1, 2, 3, 4, 5, 6, 7, 8, 9, or 10 activated carbon filtration step(s) are performed.
331. The method of any one of paragraphs 285-329, wherein 1, 2 or 3 activated carbon filtration step(s) are performed.
332. The method of any one of paragraphs 285-329, wherein 1 or 2 activated carbon filtration step(s) are performed.
333. The method of any one of paragraphs 285-332, wherein the solution is treated by activated carbon filters in series.
334. The method of any one of paragraphs 285-332, wherein the solution is treated by 1, 2, 3, 4, 5, 6, 7, 8, 9, or 10 activated carbon filters in series.
335. The method of any one of paragraphs 285-332, wherein the solution is treated by 2, 3, 4 or 5 activated carbon filters in series.
336. The method of any one of paragraphs 285-332, wherein the solution is treated by 2 activated carbon filters in series.
337. The method of any one of paragraphs 285-332, wherein the solution is treated by 3 activated carbon filters in series.
338. The method of any one of paragraphs 285-332, wherein the solution is treated by 4 activated carbon filters in series.
339. The method of any one of paragraphs 285-332, wherein the solution is treated by 5 activated carbon filters in series.
340. The method of any one of paragraphs 285-339, wherein the activated carbon filtration step is performed in a single pass mode.
341. The method of any one of paragraphs 285-339, wherein the activated carbon filtration step is performed in recirculation mode.
342. The method of paragraph 341, wherein 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 cycles of activated carbon filtration are performed.
343. The method of paragraph 341, wherein 2, 3, 4, 5, 6, 7, 8, 9 or 10 cycles of activated carbon filtration are performed.
344. The method of paragraph 341, wherein 2 or 3 cycles of activated carbon filtration are performed.
345. The method of paragraph 341, wherein 2 cycles of activated carbon filtration are performed.
346. The method of any one of paragraphs 285-345, whereinthe filtrate is further filtered.
347. The method of any one of paragraphs 285-345, wherein the filtrate is subjected to microfiltration.
348. The method of paragraph 347, wherein said microfiltration is dead-end filtration (perpendicular filtration).
349. The method of paragraph 347, wherein said microfiltration is tangential microfiltration.
350. The method of any one of paragraphs 347-349, wherein said microfiltration filter has a nominal retention range of between about 0.01-2 micron, about 0.05-2 micron, about 0.1-2 micron, about 0.2-2 micron, about 0.3-2 micron, about 0.4-2 micron, about 0.45-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, or about 1.75-2 micron.
351. The method of any one of paragraphs 347-349, wherein said microfiltration filter has a nominal retention range of between about 0.01-1 micron, about 0.05-1 micron, about 0.1-1 micron, about 0.2-1 micron, about 0.3-1 micron, about 0.4-1 micron, about 0.45-1 micron, about 0.5-1 micron, about 0.6-1 micron, about 0.7-1 micron, about 0.8-1 micron or about 0.9-1 micron.
352. be method of any one of paragraphs 347-349, wherein said microfiltration filter has a nominal retention range of about 0.01, about 0.05, about 0.1, about 0.2, about 0.3, about 0.4, about 0.45, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9 or about 2.0 micron.
353. The method of any one of paragraphs 343-345, wherein said microfiltration filter has a nominal retention range of about 0.2 micron.
354. The method of any one of paragraphs 347-353, wherein said microfiltration filter has a filter capacity of 100-6000 L/m², 200-6000 L/m², 300-6000 L/m², 400-6000 L/m², 500-6000 L/m², 750-6000 L/m², 1000-6000 L/m², 1500-6000 L/m², 2000-6000 L/m², 3000-6000 L/m² or 4000-6000 L/m².
355. The method of any one of paragraphs 347-353, wherein said microfiltration filter has a filter capacity of 100-4000 L/m², 200-4000 L/m², 300-4000 L/m², 400-4000 L/m², 500-4000 L/m², 750-4000 L/m², 1000-4000 L/m², 1500-4000 L/m², 2000-4000 L/m², 2500-4000 L/m², 3000-4000 L/m², 3000-4000 L/m² or 3500-4000 L/m².
356. The method of any one of paragraphs 347-353, wherein said microfiltration filter has a filter capacity of 100-3750 L/m², 200-3750 L/m², 300-3750 L/m², 400-3750 L/m², 500-3750 L/m², 750-3750 L/m², 1000-3750 L/m², 1500-3750 L/m², 2000-3750 L/m², 2500-3750 L/m², 3000-3750 L/m², 3000-3750 L/m² or 3500-3750 L/m².
357. The method of any one of paragraphs 347-353, wherein said microfiltration filter has a filter capacity of 100-1250 L/m², 200-1250 L/m², 300-1250 L/m², 400-1250 L/m², 500-1250 L/m², 750-1250 L/m² or 1000-1250 L/m².
358. The method of any one of paragraphs 347-353, wherein said microfiltration filter has a filter capacity of about 100, about 200, about 300, about 400, about 550, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5250, about 5500, about 5750 or about 6000 L/m².
359. The method of any one of paragraphs 285-359, wherein the filtrate is further clarified by ultrafiltration and/or dialfiltration.
360. The method of any one of paragraphs 285-359, wherein the filtrate is further clarified by ultrafiltration.
361. The methods of paragraph 359 or 360 wherein the molecular weight cut off of said ultrafiltration membrane is in the range of between about 5 kDa-1000 kDa.
362. The methods of paragraph 359 or 360 wherein the molecular weight cut off of said ultrafiltration membrane is in the range of between about 10 kDa-750 kDa.
363. The methods of paragraph 359 or 360 wherein the molecular weight cut off of said ultrafiltration membrane is in the range of between about 10 kDa-500 kDa.
364. The methods of paragraph 359 or 360 wherein the molecular weight cut off of said ultrafiltration membrane is in the range of between about 10 kDa-300 kDa.
365. The methods of paragraph 359 or 360 wherein the molecular weight cut off of said ultrafiltration membrane is in the range of between about 10 kDa-100 kDa.
366. The methods of paragraph 359 or 360 wherein the molecular weight cut off of said ultrafiltration membrane is in the range of between about 10 kDa-50 kDa.
367. The methods of paragraph 359 or 360 wherein the molecular weight cut off of said ultrafiltration membrane is in the range of between about 10 kDa-30 kDa.
368. The methods of paragraph 359 or 360 wherein the molecular weight cut off of said ultrafiltration membrane is in the range of between about 5 kDa-1000 kDa, about 10 kDa-1000 kDa about 20 kDa-1000 kDa, about 30 kDa-1000 kDa, about 40 kDa-1000 kDa, about 50 kDa-1000 kDa, about 75 kDa-1000 kDa, about 100 kDa-1000 kDa, about 150 kDa-1000 kDa, about 200 kDa-1000 kDa, about 300 kDa-1000 kDa, about 400 kDa-1000 kDa, about 500 kDa-1000 kDa or about 750 kDa-1000 kDa.
369. The methods of paragraph 359 or 360 wherein the molecular weight cut off of said ultrafiltration membrane is in the range of between about 5 kDa-500 kDa, about 10 kDa-500 kDa, about 20 kDa-500 kDa, about 30 kDa-500 kDa, about 40 kDa-500 kDa, about 50 kDa-500 kDa, about 75 kDa-500 kDa, about 100 kDa-500 kDa, about 150 kDa-500 kDa, about 200 kDa-500 kDa, about 300 kDa-500 kDa or about 400 kDa-500 kDa.
370. The methods of paragraph 359 or 360 wherein the molecular weight cut off of said ultrafiltration membrane is in the range of between about 5 kDa-300 kDa, about 10 kDa-300 kDa, about 20 kDa-300 kDa, about 30 kDa-300 kDa, about 40 kDa-300 kDa, about 50 kDa-300 kDa, about 75 kDa-300 kDa, about 100 kDa-300 kDa, about 150 kDa-300 kDa or about 200 kDa-300 kDa.
371. The methods of paragraph 359 or 360 wherein the molecular weight cut off of said ultrafiltration membrane is in the range of between about 5 kDa-100 kDa, about 10 kDa-100 kDa, about 20 kDa-100 kDa, about 30 kDa-100 kDa, about 40 kDa-100 kDa, about 50 kDa-100 kDa or about 75 kDa-100 kDa.
372. The methods of paragraph 359 or 360 wherein the molecular weight cut off of said ultrafiltration membrane is about 5 kDa, about 10 kDa, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 200 kDa, about 250 kDa, about 300 kDa, about 400 kDa, about 500 kDa, about 750 kDa or about 1000 kDa.
373. The methods of any one of paragraph 359-371 wherein the concentration factor of said ultrafiltration step is from about 1.5 to about 10.0.
374. The methods of any one of paragraph 359-371 wherein the concentration factor of said ultrafiltration step is from about 2.0 to about 8.0.
375. The methods of any one of paragraph 359-371 wherein the concentration factor of said ultrafiltration step is from about 2.0 to about 5.0.
376. The methods of any one of paragraph 359-371 wherein the concentration factor of said ultrafiltration step is about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0. In an embodiment, the concentration factor is about 2.0, about 3.0, about 4.0, about 5.0, or about 6.0.
377. The method of any one of paragraphs 359-376 wherein said ultrafiltration step is performed at temperature between about 20° C. to about 90° C.
378. The method of any one of paragraphs 359-376 wherein said ultrafiltration step is performed at temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
379. The method of any one of paragraphs 359-376 wherein said ultrafiltration step is performed at temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
380. The method of any one of paragraphs 359-376 wherein said ultrafiltration step is performed at temperature of about 50° C.
381. The method of any one of paragraphs 359-380 wherein the ultrafiltration filtrate is treated by diafiltration.
382. The method of paragraphs 381 wherein the replacement solution is water.
383. The method of paragraph 381 wherein the replacement solution is saline in water.
384. The method of paragraph 383 wherein the salt is selected from the group consisting of magnesium chloride, potassium chloride, sodium chloride and a combination thereof.
385. The method of paragraphs 383 wherein the salt is sodium chloride.
386. The method of paragraphs 383 wherein the replacement solution is sodium chloride at about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM or about 500 mM.
387. The method of paragraph 381 wherein the replacement solution is a buffer solution.
388. The method of paragraph 381 wherein the replacement solution is a buffer solution wherein the buffer is selected from the group consisting of N-(2-Acetamido)-aminoethanesulfonic acid (ACES), a salt of acetic acid (acetate), N-(2-Acetamido)-iminodiacetic acid (ADA), 2-Aminoethanesulfonic acid (AES, Taurine), ammonia, 2-Amino-2-methyl-1-propanol (AMP), 2-Amino-2-methyl-1,3-propanediol AMPD, ammediol, N-(1,1-Dimethyl-2-hydroxyethyl)-3-amino-2-hydroxypropanesulfonic acid (AMPSO), N,N-Bis-(2-hydroxyethyl)-2-aminoethanesulfonic acid (BES), sodium hydrogen carbonate (bicarbonate), N,N′-Bis(2-hydroxyethyl)-glycine (bicine), [Bis-(2-hydroxyethyl)-imino]-tris-(hydroxymethylmethane) (BIS-Tris), 1,3-Bis[tris(hydroxymethyl)-methylamino]propane (BIS-Tris-Propane), Boric acid, dimethylarsinic acid (Cacodylate), 3-(Cyclohexylamino)-propanesulfonic acid (CAPS), 3-(Cyclohexylamino)-2-hydroxy-1-propanesulfonic acid (CAPSO), sodium carbonate (Carbonate), cyclohexylaminoethanesulfonic acid (CHES), a salt of citric acid (citrate), 3[N-Bis(hydroxyethyl)amino]-2-hydroxypropanesulfonic acid (DIPSO), a salt of formic acid (formate), Glycine, Glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, a salt of malic acid (Malate), a salt of maleic acid (Maleate), 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), a salt of phosphoric acid (Phosphate), Piperazine-N,N′-bis(2-ethanesulfonic acid) (PIPES), Piperazine-N,N′-bis(2-hydroxypropanesulfonic acid) (POPSO), pyridine, a salt of succinic acid (Succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine) and Tris(hydroxymethyl)-aminomethane (Tris).
389. The method of paragraph 381 wherein the replacement solution is a buffer solution wherein the buffer is selected from the group consisting of a salt of acetic acid (acetate), a salt of citric acid (citrate), a salt of formic acid (formate), a salt of malic acid (Malate), a salt of maleic acid (Maleate), a salt of phosphoric acid (Phosphate) and a salt of succinic acid (Succinate).
390. The method of paragraph 381 wherein the replacement solution is a buffer solution wherein the buffer is a salt of citric acid (citrate).
391. The method of paragraph 381 wherein the replacement solution is a buffer solution wherein the buffer is a salt of succinic acid (succinate).
392. The method of paragraph 381 wherein the replacement solution is a buffer solution wherein the buffer is a salt of phosphoric acid (phosphate).
393. The method of any one of paragraphs 388-392 said salt is a sodium salt.
394. The method of any one of paragraphs 388-392 said salt is a potassium salt.
395. The method of paragraph 381 wherein the replacement solution is a buffer solution wherein the buffer is potassium phosphate.
396. The method of any one of paragraphs 381-395 wherein the pH of the diafiltration buffer is between about 4.0-11.0, between about 5.0-10.0, between about 5.5-9.0, between about 6.0-8.0, between about 6.0-7.0, between about 6.5-7.5, between about 6.5-7.0 or between about 6.0-7.5.
397. The method of paragraph 381-395 wherein the pH of the diafiltration buffer is about 4.0, about 4.5, about 5.0, about 5.5, about 6.0, about 6.5, about 7.0, about 7.5, about 8.0, about 8.5, about 9.0, about 9.5, about 10.0, about 10.5 or about 11.0.
398. The method of any one of paragraphs 381-395 wherein the pH of the diafiltration buffer is about 6.0, about 6.5, about 7.0, about 7.5, about 8.0, about 8.5 or about 9.0.
399. The method of any one of paragraphs 381-395 wherein the pH of the diafiltration buffer is about 6.5, about 7.0 or about 7.5.
400. The method of any one of paragraphs 381-395 wherein the pH of the diafiltration buffer is about 6.0.
401. The method of any one of paragraphs 381-395 wherein the pH of the diafiltration buffer is about 6.5.
402. The method of any one of paragraphs 381-395 wherein the pH of the diafiltration buffer is about 7.0
403. The method of any one of paragraphs 387-402 wherein the concentration of the diafiltration buffer is between about 0.01 mM-100 mM, between about 0.1 mM-100 mM, between about 0.5 mM-100 mM, between about 1 mM-100 mM, between about 2 mM-100 mM, between about 3 mM-100 mM, between about 4 mM-100 mM, between about 5 mM-100 mM, between about 6 mM-100 mM, between about 7 mM-100 mM, between about 8 mM-100 mM, between about 9 mM-100 mM, between about 10 mM-100 mM, between about 11 mM-100 mM, between about 12 mM-100 mM, between about 13 mM-100 mM, between about 14 mM-100 mM, between about 15 mM-100 mM, between about 16 mM-100 mM, between about 17 mM-100 mM, between about 18 mM-100 mM, between about 19 mM-100 mM, between about 20 mM-100 mM, between about 25 mM-100 mM, between about 30 mM-100 mM, between about 35 mM-100 mM, between about 40 mM-100 mM, between about 45 mM-100 mM, between about 50 mM-100 mM, between about 55 mM-100 mM, between about 60 mM-100 mM, between about 65 mM-100 mM, between about 70 mM-100 mM, between about 75 mM-100 mM, between about 80 mM-100 mM, between about 85 mM-100 mM, between about 90 mM-100 mM or between about 95 mM-100 mM.
404. The method of any one of paragraphs 387-402 wherein the concentration of the diafiltration buffer is between about 0.01 mM-50 mM, between about 0.1 mM-50 mM, between about 0.5 mM-50 mM, between about 1 mM-50 mM, between about 2 mM-50 mM, between about 3 mM-50 mM, between about 4 mM-50 mM, between about 5 mM-50 mM, between about 6 mM-50 mM, between about 7 mM-50 mM, between about 8 mM-50 mM, between about 9 mM-50 mM, between about 10 mM-50 mM, between about 11 mM-50 mM, between about 12 mM-50 mM, between about 13 mM-50 mM, between about 14 mM-50 mM, between about 15 mM-50 mM, between about 16 mM-50 mM, between about 17 mM-50 mM, between about 18 mM-50 mM, between about 19 mM-50 mM, between about 20 mM-50 mM, between about 25 mM-50 mM, between about 30 mM-50 mM, between about 35 mM-50 mM, between about 40 mM-50 mM or between about 45 mM-50 mM.
405. The method of any one of paragraphs 387-402 wherein the concentration of the diafiltration buffer is between about 0.01 mM-25 mM, between about 0.1 mM-25 mM, between about 0.5 mM-25 mM, between about 1 mM-25 mM, between about 2 mM-25 mM, between about 3 mM-25 mM, between about 4 mM-25 mM, between about 5 mM-25 mM, between about 6 mM-25 mM, between about 7 mM-25 mM, between about 8 mM-25 mM, between about 9 mM-25 mM, between about 10 mM-25 mM, between about 11 mM-25 mM, between about 12 mM-25 mM, between about 13 mM-25 mM, between about 14 mM-25 mM, between about 15 mM-25 mM, between about 16 mM-25 mM, between about 17 mM-25 mM, between about 18 mM-25 mM, between about 19 mM-25 mM or between about 20 mM-25 mM.
406. The method of any one of paragraphs 387-402 wherein the concentration of the diafiltration buffer is between about 0.01 mM-15 mM, between about 0.1 mM-15 mM, between about 0.5 mM-15 mM, between about 1 mM-15 mM, between about 2 mM-15 mM, between about 3 mM-15 mM, between about 4 mM-15 mM, between about 5 mM-15 mM, between about 6 mM-15 mM, between about 7 mM-15 mM, between about 8 mM-15 mM, between about 9 mM-15 mM, between about 10 mM-15 mM, between about 11 mM-15 mM, between about 12 mM-15 mM, between about 13 mM-15 mM or between about 14 mM-15 mM.
407. The method of any one of paragraphs 387-402 wherein the concentration of the diafiltration buffer is between about 0.01 mM-10 mM, between about 0.1 mM-10 mM, between about 0.5 mM-10 mM, between about 1 mM-10 mM, between about 2 mM-10 mM, between about 3 mM-10 mM, between about 4 mM-10 mM, between about 5 mM-10 mM, between about 6 mM-10 mM, between about 7 mM-10 mM, between about 8 mM-10 mM or between about 9 mM-10 mM.
408. The method of any one of paragraphs 387-402 wherein the concentration of the diafiltration buffer is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.
409. The method of any one of paragraphs 387-402 wherein the concentration of the diafiltration buffer is about 0.1 mM, about 0.2 mM, about 1 mM, about 5 mM, about 10 mM, about 15 mM, about 20 mM, about 30 mM, about 40 mM, or about 50 mM.
410. The method of any one of paragraphs 387-402 wherein the concentration of the diafiltration buffer is about 30 mM.
411. The method of any one of paragraphs 387-402 wherein the concentration of the diafiltration buffer is about 25 mM.
412. The method of any one of paragraphs 387-402 wherein the concentration of the diafiltration buffer is about 20 mM.
413. The method of any one of paragraphs 387-402 wherein the concentration of the diafiltration buffer is about 15 mM.
414. The method of any one of paragraphs 387-402 wherein the concentration of the diafiltration buffer is about 10 mM.
415. The method of any one of paragraphs 381-414 wherein the replacement solution comprises a chelating agent.
416. The method of any one of paragraphs 381-414 wherein the replacement solution comprises an alum chelating agent.
417. The method of any one of paragraphs 381-414 wherein the replacement solution comprises a chelating agent selected from the groups consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N,N,N′,N′-tetraacetic acid (DPTA-OH), ethylenediamine-N,N′-bis(2-hydroxyphenylacetic acid) (EDDHA), ethylenediamine-N,N′-dipropionic acid dihydrochloride (EDDP), ethylenediamine-tetrakis(methylenesulfonic acid) (EDTPO), Nitrilotris(methylenephosphonic acid) (NTPO), imino-diacetic acid (IDA), hydroxyimino-diacetic acid (HIDA), nitrilo-triacetic acid (NTP), triethylenetetramine-hexaacetic acid (TTHA), Dimercaptosuccinic acid (DMSA), 2,3-dimercapto-1-propanesulfonic acid (DMPS), alpha lipoic acid (ALA), Nitrilotriacetic acid (NTA), thiamine tetrahydrofurfuryl disulfide (TTFD), dimercaprol, penicillamine, deferoxamine (DFOA), deferasirox, phosphonates, a salt of citric acid (citrate) and combinations of these.
418. The method of any one of paragraphs 381-414 wherein the replacement solution comprises a chelating agent selected from the groups consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N,N,N′,N′-tetraacetic acid (DPTA-OH), ethylenediamine-N,N′-bis(2-hydroxyphenylacetic acid) (EDDHA), a salt of citric acid (citrate) and combinations of these.
419. The method of any one of paragraphs 381-414 wherein the replacement solution comprises Ethylene Diamine Tetra Acetate (EDTA) as chelating agent.
420. The method of any one of paragraphs 381-414 wherein the replacement solution comprises a salt of citric acid (citrate) as chelating agent.
421. The method of any one of paragraphs 381-414 wherein the replacement solution comprises sodium citrate as chelating agent.
422. The method of any one of paragraphs 415-421 wherein the concentration of the chelating agent in the replacement solution is from 1 to 500 mM.
423. The method of any one of paragraphs 415-421 wherein the concentration of the chelating agent in the replacement solution is from 2 to 400 mM.
424. The method of any one of paragraphs 415-421 wherein concentration of the chelating agent in the replacement solution is from 10 to 400 mM.
425. The method of any one of paragraphs 415-421 wherein concentration of the chelating agent in the replacement solution is from 10 to 200 mM.
426. The method of any one of paragraphs 415-421 wherein concentration of the chelating agent in the replacement solution is from 10 to 100 mM.
427. The method of any one of paragraphs 415-421 wherein concentration of the chelating agent in the replacement solution is from 10 to 50 mM.
428. The method of any one of paragraphs 415-421 wherein concentration of the chelating agent in the replacement solution is from 10 to 30 mM.
429. The method of any one of paragraphs 415-421 wherein concentration of the chelating agent in the replacement solution is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.
430. The method of any one of paragraphs 415-421 wherein concentration of the chelating agent in the replacement solution is about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM or about 100 mM.
431. The method of any one of paragraphs 415-421 wherein concentration of the chelating agent in the replacement solution is about 15 mM, about 20 mM, about 25 mM, about 30 mM, about 35 mM, about 40 mM, about 45 mM or about 50 mM.
432. The method of any one of paragraphs 387-431 wherein the replacement solution comprises a salt.
433. The method of paragraph 432 wherein, the salt is selected from the groups consisting of magnesium chloride, potassium chloride, sodium chloride and a combination thereof.
434. The method of paragraph 432 wherein, the salt is sodium chloride.
435. The method of any one of paragraphs 432-434 wherein the replacement solution comprises sodium chloride at 1 about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250 or about 300 mM.
436. The method of any one of paragraphs 381-435 wherein the number of diavolumes is at least 5, 10, 15, 20, 25, 30, 35, 40, 45, or 50.
437. The method of any one of paragraphs 381-435 wherein the number of diavolumes is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95 or about 100.
438. The method of any one of paragraphs 381-435 wherein the number of diavolumes is about 5, about 6, about 7, about 8, about 9, about 10, about 11, about 12, about 13, about 14 or about 15.
439. The method of any one of paragraphs 381-438 wherein said dialfiltration step is performed at temperature of between about 20° C. to about 90° C.
440. The method of any one of paragraphs 381-438 wherein said dialfiltration step is performed at temperature of between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
441. The method of any one of paragraphs 381-438 wherein said dialfiltration step is performed at temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
442. The method of any one of paragraphs 381-438 wherein said dialfiltration step is performed at temperature of about 50° C.
443. The method of any one of paragraphs 359-438 wherein said ultrafiltration and dialfiltration steps if both conducted are performed at a temperature between about 20° C. to about 90° C.
444. The method of any one of paragraphs 359-438 wherein said ultrafiltration and dialfiltration steps if both conducted are performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
445. The method of any one of paragraphs 359-438 wherein said ultrafiltration and dialfiltration steps if both conducted are performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
446. The method of any one of paragraphs 359-438 wherein said ultrafiltration and dialfiltration steps if both conducted are performed at a temperature of about 50° C.
447. The method of any one of paragraphs 359-446 wherein said purified solution of polysaccharide is homogenized by sizing.
448. The method of any one of paragraphs 359-446 wherein said purified solution of polysaccharide is subjected to mechanical sizing.
449. The method of any one of paragraphs 359-446 wherein said purified solution of polysaccharide is subjected to High Pressure Homogenization Shearing.
450. The method of any one of paragraphs 359-446 wherein said purified solution of polysaccharide is subjected to chemical hydrolysis.
451. The method of any one of paragraphs 359-450 wherein said purified solution of polysaccharide is sized to a target molecular weight.
452. The method of any one of paragraphs 359-451 wherein said purified solution of polysaccharide is sized to a molecular weight of between about 5 kDa and about 4,000 kDa.
453. The method of any one of paragraphs 359-451 wherein said purified solution of polysaccharide is sized to a molecular weight of between about 10 kDa and about 4,000 kDa.
454. The method of any one of paragraphs 359-451 wherein said purified solution of polysaccharide is sized to a molecular weight of between about 50 kDa and about 4,000 kDa.
455. The method of any one of paragraphs 359-451 wherein said purified solution of polysaccharide is sized to a molecular weight of between about 50 kDa and about 3,500 kDa; between about 50 kDa and about 3,000 kDa; between about 50 kDa and about 2,500 kDa; between about 50 kDa and about 2,000 kDa; between about 50 kDa and about 1,750 kDa; about between about 50 kDa and about 1,500 kDa; between about 50 kDa and about 1,250 kDa; between about 50 kDa and about 1,000 kDa; between about 50 kDa and about 750 kDa; between about 50 kDa and about 500 kDa; between about 100 kDa and about 4,000 kDa; between about 100 kDa and about 3,500 kDa; about 100 kDa and about 3,000 kDa; about 100 kDa and about 2,500 kDa; about 100 kDa and about 2,250 kDa; between about 100 kDa and about 2,000 kDa; between about 100 kDa and about 1,750 kDa; between about 100 kDa and about 1,500 kDa; between about 100 kDa and about 1,250 kDa; between about 100 kDa and about 1,000 kDa; between about 100 kDa and about 750 kDa; between about 100 kDa and about 500 kDa; between about 200 kDa and about 4,000 kDa; between about 200 kDa and about 3,500 kDa; between about 200 kDa and about 3,000 kDa; between about 200 kDa and about 2,500 kDa; between about 200 kDa and about 2,250 kDa; between about 200 kDa and about 2,000 kDa; between about 200 kDa and about 1,750 kDa; between about 200 kDa and about 1,500 kDa; between about 200 kDa and about 1,250 kDa; between about 200 kDa and about 1,000 kDa; between about 200 kDa and about 750 kDa; or between about 200 kDa and about 500 kDa. In further such embodiments, the polysaccharide the purified polysaccharide is sized to a molecular weight of between about 250 kDa and about 3,500 kDa; between about 250 kDa and about 3,000 kDa; between about 250 kDa and about 2,500 kDa; between about 250 kDa and about 2,000 kDa; between about 250 kDa and about 1,750 kDa; about between about 250 kDa and about 1,500 kDa; between about 250 kDa and about 1,250 kDa; between about 250 kDa and about 1,000 kDa; between about 250 kDa and about 750 kDa; between about 250 kDa and about 500 kDa; between about 300 kDa and about 4,000 kDa; between about 300 kDa and about 3,500 kDa; about 300 kDa and about 3,000 kDa; about 300 kDa and about 2,500 kDa; about 300 kDa and about 2,250 kDa; between about 300 kDa and about 2,000 kDa; between about 300 kDa and about 1,750 kDa; between about 300 kDa and about 1,500 kDa; between about 300 kDa and about 1,250 kDa; between about 300 kDa and about 1,000 kDa; between about 300 kDa and about 750 kDa; between about 300 kDa and about 500 kDa; between about 500 kDa and about 4,000 kDa; between about 500 kDa and about 3,500 kDa; between about 500 kDa and about 3,000 kDa; between about 500 kDa and about 2,500 kDa; between about 500 kDa and about 2,250 kDa; between about 500 kDa and about 2,000 kDa; between about 500 kDa and about 1,750 kDa; between about 500 kDa and about 1,500 kDa; between about 500 kDa and about 1,250 kDa; between about 500 kDa and about 1,000 kDa; between about 500 kDa and about 750 kDa; or between about 500 kDa and about 600 kDa.
456. The method of any one of paragraphs 359-451 wherein said purified solution of polysaccharide is sized to a molecular weight of about 5 kDa, about 10 kDa, about 15 kDa, about 20 kDa, about 25 kDa, about 30 kDa, about 35 kDa, about 40 kDa, about 45 kDa, about 50 kDa, about 75 kDa, about 90 kDa, about 100 kDa, about 150 kDa, about 200 kDa, about 250 kDa, about 300 kDa, about 350 kDa, about 400 kDa, about 450 kDa, about 500 kDa, about 550 kDa, about 600 kDa, about 650 kDa, about 700 kDa, about 750 kDa, about 800 kDa, about 850 kDa, about 900 kDa, about 950 kDa, about 1000 kDa, about 1250 kDa, about 1500 kDa, about 1750 kDa, about 2000 kDa, about 2250 kDa, about 2500 kDa, about 2750 kDa, about 3000 kDa, about 3250 kDa, about 3500 kDa, about 3750 kDa or about 4,000 kDa.
457. The method of any one of paragraphs 1-456 wherein said purified solution of polysaccharide is sterilely filtered.
458. The method of paragraph 457 wherein said sterile filtration is dead-end filtration.
459. The method of paragraph 457 wherein said sterile filtration is tangential filtration.
460. The method of any one of paragraphs 457-459 wherein the filter has a nominal retention range of between about 0.01-0.2 micron, about 0.05-0.2 micron, about 0.1-0.2 micron or about 0.15-0.2 micron.
461. The method of any one of paragraphs 457-459 wherein the filter has a nominal retention range of about 0.05, about 0.1, about 0.15 or about 0.2 micron.
462. The method of any one of paragraphs 457-459 wherein the filter has a nominal retention range of about 0.2 micron.
463. The method of any one of paragraphs 457-462 wherein the filter has a filter capacity of about 25-1500 L/m², 50-1500 L/m², 75-1500 L/m², 100-1500 L/m², 150-1500 L/m², 200-1500 L/m², 250-1500 L/m², 300-1500 L/m², 350-1500 L/m², 400-1500 L/m², 500-1500 L/m², 750-1500 L/m², 1000-1500 L/m² or 1250-1500 L/m².
464. The method of any one of paragraphs 457-462 wherein the filter has a filter capacity of about 25-1000 L/m², 50-1000 L/m², 75-1000 L/m², 100-1000 L/m², 150-1000 L/m², 200-1000 L/m², 250-1000 L/m², 300-1000 L/m², 350-1000 L/m², 400-1000 L/m², 500-1000 L/m² or 750-1000 L/m².
465. The method of any one of paragraphs 457-462 wherein the filter has a filter capacity of a filter capacity of 25-500 L/m², 50-500 L/m², 75-500 L/m², 100-500 L/m², 150-500 L/m², 200-500 L/m², 250-500 L/m², 300-500 L/m², 350-500 L/m² or 400-500 L/m².
466. The method of any one of paragraphs 457-462 wherein the filter has a filter capacity of 25-300 L/m², 50-300 L/m², 75-300 L/m², 100-300 L/m², 150-300 L/m², 200-300 L/m² or 250-300 L/m².
467. The method of any one of paragraphs 457-462 wherein the filter has a filter capacity of 25-250 L/m², 50-250 L/m², 75-250 L/m², 100-250 L/m² or 150-250 L/m², 200-250 L/m².
468. The method of any one of paragraphs 457-462 wherein the filter has a filter capacity of 25-100 L/m², 50-100 L/m² or 75-100 L/m².
469. The method of any one of paragraphs 457-462 wherein the filter has a filter capacity of about 25, about 50, about 75, about 100, about 150, about 200, about 250, about 300, about 350, about 400, about 500, about 600, about 700, about 800, about 900, about 1000, about 1100, about 1200, about 1300, about 1400 or about 1500 L/m².
470. The method of any one of paragraphs 1-469 wherein the obtained purified polysaccharide is in liquid solution.
471. The method of any one of paragraphs 1-469 wherein the obtained purified polysaccharide is a dried powder.
472. The method of any one of paragraphs 1-469 wherein the obtained purified polysaccharide solution is lyophilized.
473. The method of any one of paragraphs 1-469 or 472 wherein the obtained purified polysaccharide solution is a freeze-dried cake.
474. The method of any one of paragraphs 1-473 wherein said bacterial polysaccharide is a capsular polysaccharide, a sub-capsular polysaccharide, or a lipopolysaccharide.
475. The method of any one of paragraphs 1-473 wherein said bacterial polysaccharide is a capsular polysaccharide.
476. The method of any one of paragraphs 1-473 wherein said bacterial polysaccharide is a capsular polysaccharide from Staphylococcus aureus.
477. The method of any one of paragraphs 1-473 said bacterial polysaccharide is the capsular polysaccharide from Staphylococcus aureus type 5.
478. The method of any one of paragraphs 1-473 wherein said bacterial polysaccharide is the capsular polysaccharide from Staphylococcus aureus type 8.
479. The method of any one of paragraphs 1-473 wherein said bacterial polysaccharide is a capsular polysaccharide from Enterococcus faecalis.
480. The method of any one of paragraphs 1-473 wherein said bacterial polysaccharide is the capsular polysaccharide from Haemophilus influenzae type b.
481. The method of any one of paragraphs 1-473 wherein said bacterial polysaccharide is a capsular polysaccharide from Neisseria meningitidis.
482. The method of any one of paragraphs 1-473 wherein said bacterial polysaccharide is the capsular polysaccharide from N. meningitidis serogroup A (MenA), N. meningitidis serogroup W135 (MenW135), N. meningitidis serogroup Y (MenY), N. meningitidis serogroup X (MenX) or N. meningitidis serogroup C (MenC).
483. The method of any one of paragraphs 1-473 wherein said bacterial polysaccharide is a capsular polysaccharide from Escherichia coli.
484. The method of any one of paragraphs 1-473 wherein said bacterial polysaccharide is a capsular polysaccharide from Streptococcus agalactiae (Group B streptococcus (GBS)).
485. The method of any one of paragraphs 1-473 wherein said bacterial polysaccharide is a capsular polysaccharide selected from the group consisting of the capsular polysaccharide from GBS types Ia, Ib, II, III, IV, V, VI, VII and VIII.
486. The method of any one of paragraphs 1-473 wherein said bacterial polysaccharide is a capsular polysaccharide from an Escherichia coli strain part of the Enterovirulent Escherichia coli group (EEC Group).
487. The method of any one of paragraphs 1-473 wherein said bacterial polysaccharide is a capsular polysaccharide from an Escherichia coli strain part of the Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), or Escherichia coli—enteroinvasive (EIEC). In an embodiment, the source of bacterial capsular polysaccharide is an Uropathogenic Escherichia coli (UPEC).
488. The method of any one of paragraphs 1-473 wherein said bacterial polysaccharide is a capsular polysaccharide from an Escherichia coli serotype selected from the group consisting of serotypes O157:H7, O26:H11, O111:H- and O103:H2.
489. The method of any one of paragraphs 1-473 wherein said bacterial polysaccharide is a capsular polysaccharide from an Escherichia coli serotype selected from the group consisting of serotypes O6:K2:H1 and O18:K1:H7.
490. The method of any one of paragraphs 1-473 wherein said bacterial polysaccharide is a capsular polysaccharide from an Escherichia coli serotype selected from the group consisting of serotypes O45:K1, O17:K52:H18, O19:H34 and O7:K1.
491. The method of any one of paragraphs 1-473 wherein said bacterial polysaccharide is a capsular polysaccharide from an Escherichia coli serotype 0104: H 4.
492. The method of any one of paragraphs 1-473 wherein said bacterial polysaccharide is a capsular polysaccharide from an Escherichia coli serotype O1:K12:H7.
493. The method of any one of paragraphs 1-473 wherein said bacterial polysaccharide is a capsular polysaccharide from an Escherichia coli serotype O127:H6.
494. The method of any one of paragraphs 1-473 wherein said bacterial polysaccharide is a capsular polysaccharide from an Escherichia coli serotype O139:H28.
495. The method of any one of paragraphs 1-473 wherein said bacterial polysaccharide is a capsular polysaccharide from an Escherichia coli serotype O128:H2.
496. The method of any one of paragraphs 1-473 wherein said bacterial polysaccharide is a capsular polysaccharide from Steptococcus pneumoniae.
497. The method of any one of paragraphs 1-473 wherein said bacterial polysaccharide is the capsular polysaccharide from a Streptococcus pneumoniae serotype selected from the group consisting of serotypes 1, 2, 3, 4, 5, 6A, 6B, 6C, 7F, 8, 9V, 9N, 10A, 11A, 12F, 14, 15A, 15B, 15C, 16F, 17F, 18C, 19A, 19F, 20, 22F, 23A, 23B, 23F, 24B, 24F, 29, 31, 33F, 34, 35B, 35F, 38, 72 and 73.
498. The method of any one of paragraphs 1-473 wherein said bacterial polysaccharide is the capsular polysaccharide from a Streptococcus pneumoniae serotype selected from the group consisting of serotypes 1, 2, 3, 4, 5, 6A, 6B, 7F, 8, 9V, 9N, 10A, 11A, 12F, 14, 15A, 15B, 15C, 16F, 17F, 18C, 19A, 19F, 20, 22F, 23A, 23B, 23F, 24F, 29, 31, 33F, 35B, 35F, 38, 72 and 73.
499. The method of any one of paragraphs 1-473 wherein said bacterial polysaccharide is the capsular polysaccharide from a Streptococcus pneumoniae serotype selected from the group consisting of serotypes 8, 10A, 11A, 12F, 15B, 22F and 33F.
500. The method of any one of paragraphs 1-473 wherein said bacterial polysaccharide is the capsular polysaccharide from Streptococcus pneumoniae serotype 1.
501. The method of any one of paragraphs 1-473 wherein said bacterial polysaccharide is the capsular polysaccharide from Streptococcus pneumoniae serotype 2.
502. The method of any one of paragraphs 1-473 wherein said bacterial polysaccharide is the capsular polysaccharide from Streptococcus pneumoniae serotype 3.
503. The method of any one of paragraphs 1-473 wherein said bacterial polysaccharide is the capsular polysaccharide from Streptococcus pneumoniae serotype 4.
504. The method of any one of paragraphs 1-473 wherein said bacterial polysaccharide is the capsular polysaccharide from Streptococcus pneumoniae serotype 5.
505. The method of any one of paragraphs 1-473 wherein said bacterial polysaccharide is the capsular polysaccharide from Streptococcus pneumoniae serotype 6A.
506. The method of any one of paragraphs 1-473 wherein said bacterial polysaccharide is the capsular polysaccharide from Streptococcus pneumoniae serotype 6B.
507. The method of any one of paragraphs 1-473 wherein said bacterial polysaccharide is the capsular polysaccharide from Streptococcus pneumoniae serotype 6C.
508. The method of any one of paragraphs 1-473 wherein said bacterial polysaccharide is the capsular polysaccharide from Streptococcus pneumoniae serotype 7F.
509. The method of any one of paragraphs 1-473 wherein said bacterial polysaccharide is the capsular polysaccharide from Streptococcus pneumoniae serotype 8.
510. The method of any one of paragraphs 1-473 wherein said bacterial polysaccharide is the capsular polysaccharide from Streptococcus pneumoniae serotype 9V.
511. The method of any one of paragraphs 1-473 wherein said bacterial polysaccharide is the capsular polysaccharide from Streptococcus pneumoniae serotype 9N.
512. The method of any one of paragraphs 1-473 wherein said bacterial polysaccharide is the capsular polysaccharide from Streptococcus pneumoniae serotype 10A.
513. The method of any one of paragraphs 1-473 wherein said bacterial polysaccharide is the capsular polysaccharide from Streptococcus pneumoniae serotype 11A.
514. The method of any one of paragraphs 1-473 wherein said bacterial polysaccharide is the capsular polysaccharide from Streptococcus pneumoniae serotype 12F.
515. The method of any one of paragraphs 1-473 wherein said bacterial polysaccharide is the capsular polysaccharide from Streptococcus pneumoniae serotype 14.
516. The method of any one of paragraphs 1-473 wherein said bacterial polysaccharide is the capsular polysaccharide from Streptococcus pneumoniae serotype 15A.
517. The method of any one of paragraphs 1-473 wherein said bacterial polysaccharide is the capsular polysaccharide from Streptococcus pneumoniae serotype 15B.
518. The method of any one of paragraphs 1-473 wherein said bacterial polysaccharide is the capsular polysaccharide from Streptococcus pneumoniae serotype 15C.
519. The method of any one of paragraphs 1-473 wherein said bacterial polysaccharide is the capsular polysaccharide from Streptococcus pneumoniae serotype 16F.
520. The method of any one of paragraphs 1-473 wherein said bacterial polysaccharide is the capsular polysaccharide from Streptococcus pneumoniae serotype 17F.
521. The method of any one of paragraphs 1-473 wherein said bacterial polysaccharide is the capsular polysaccharide from Streptococcus pneumoniae serotype 18C.
522. The method of any one of paragraphs 1-473 wherein said bacterial polysaccharide is the capsular polysaccharide from Streptococcus pneumoniae serotype 19A.
523. The method of any one of paragraphs 1-473 wherein said bacterial polysaccharide is the capsular polysaccharide from Streptococcus pneumoniae serotype 19F.
524. The method of any one of paragraphs 1-473 wherein said bacterial polysaccharide is the capsular polysaccharide from Streptococcus pneumoniae serotype 20.
525. The method of any one of paragraphs 1-473 wherein said bacterial polysaccharide is the capsular polysaccharide from Streptococcus pneumoniae serotype 20A.
526. The method of any one of paragraphs 1-473 wherein said bacterial polysaccharide is the capsular polysaccharide from Streptococcus pneumoniae serotype 20B.
527. The method of any one of paragraphs 1-473 wherein said bacterial polysaccharide is the capsular polysaccharide from Streptococcus pneumoniae serotype 22F.
528. The method of any one of paragraphs 1-473 wherein said bacterial polysaccharide is the capsular polysaccharide from Streptococcus pneumoniae serotype 23A.
529. The method of any one of paragraphs 1-473 wherein said bacterial polysaccharide is the capsular polysaccharide from Streptococcus pneumoniae serotype 23B.
530. The method of any one of paragraphs 1-473 wherein said bacterial polysaccharide is the capsular polysaccharide from Streptococcus pneumoniae serotype 23F.
531. The method of any one of paragraphs 1-473 wherein said bacterial polysaccharide is the capsular polysaccharide from Streptococcus pneumoniae serotype 24B.
532. The method of any one of paragraphs 1-473 wherein said bacterial polysaccharide is the capsular polysaccharide from Streptococcus pneumoniae serotype 24F.
533. The method of any one of paragraphs 1-473 wherein said bacterial polysaccharide is the capsular polysaccharide from Streptococcus pneumoniae serotype 29.
534. The method of any one of paragraphs 1-473 wherein said bacterial polysaccharide is the capsular polysaccharide from Streptococcus pneumoniae serotype 31.
535. The method of any one of paragraphs 1-473 wherein said bacterial polysaccharide is the capsular polysaccharide from Streptococcus pneumoniae serotype 33F.
536. The method of any one of paragraphs 1-473 wherein said bacterial polysaccharide is the capsular polysaccharide from Streptococcus pneumoniae serotype 34.
537. The method of any one of paragraphs 1-473 wherein said bacterial polysaccharide is the capsular polysaccharide from Streptococcus pneumoniae serotype 35B.
538. The method of any one of paragraphs 1-473 wherein said bacterial polysaccharide is the capsular polysaccharide from Streptococcus pneumoniae serotype 35F.
539. The method of any one of paragraphs 1-473 wherein said bacterial polysaccharide is the capsular polysaccharide from Streptococcus pneumoniae serotype 38.
540. The method of any one of paragraphs 1-473 wherein said bacterial polysaccharide is the capsular polysaccharide from Streptococcus pneumoniae serotype 72.
541. The method of any one of paragraphs 1-473 wherein said bacterial polysaccharide is the capsular polysaccharide from Streptococcus pneumoniae serotype 73.
542. A purified bacterial polysaccharide obtained by the method of any one of paragraphs 1-541.
543. A purified bacterial polysaccharide obtainable by the method of any one of paragraphs 1-541.
544. A purified bacterial polysaccharide obtained by the method of any one of paragraphs 1-541 for use as an antigen.
545. A purified bacterial polysaccharide obtained by the method of any one of paragraphs 1-541 conjugated to carrier protein.
546. A purified bacterial polysaccharide obtained by the method of any one of paragraphs 1-541 further conjugated to a carrier protein.
547. A glycoconjugate of a purified bacterial polysaccharide obtained by the method of any one of paragraphs 1-541.
548. An immunogenic composition comprising any of the purified polysaccharide of any one of paragraphs 542-543.
549. An immunogenic composition comprising a glycoconjugate of any one of paragraphs 546-547.
550. An immunogenic composition comprising any of the glycoconjugate disclosed herein.
551. An immunogenic composition comprising any of the combination of glycoconjugates disclosed herein.

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every number within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of” and “consists of”, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

Any whole number integer within any of the ranges of the present document is contemplated as an embodiment of the disclosure.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLE

### Example 1. Purification of Pneumococcal Polysaccharide Serotype 8

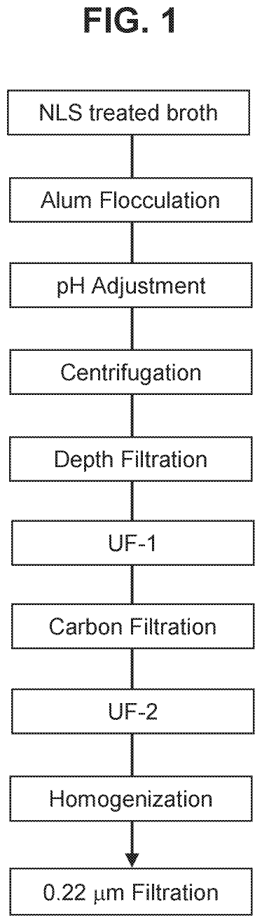

The process flow diagram for the purification is shown in FIG. 1. The process begins with NLS inactivated fermentation broth (see EP2129693) and includes recover unit operations (flocculation, centrifugation and depth filtration) followed by purification unit operations (ultrafiltration, and carbon filtration).

1. Starting Material

The process begins with NLS inactivated fermentation broth of S. pneumonia serotype 8 Cultures were grown in Hy-Soy medium. At the end of growth (as indicated by no further increase in optical density), the cultures were inactivated with NLS (see EP2129693).

2. Flocculation

The main purpose of this step is to precipitates cell debris, host cell proteins and nucleic acids. It also aids in the downstream clarification unit operations. The flocculation has been performed using fermentation broth that has been lysed by the addition of NLS.

2.1 Effect of pH and Alum

Experiments were conducted to examine the effect of pH, percent alum and hold time.

A preset amount of fermentation broth was aliquot into different containers, and a 10% (w/w) stock alum solution (prepared using aluminum potassium sulfate dodecahydrate and deionized water) was added to a final concentration of 2% (w/v).

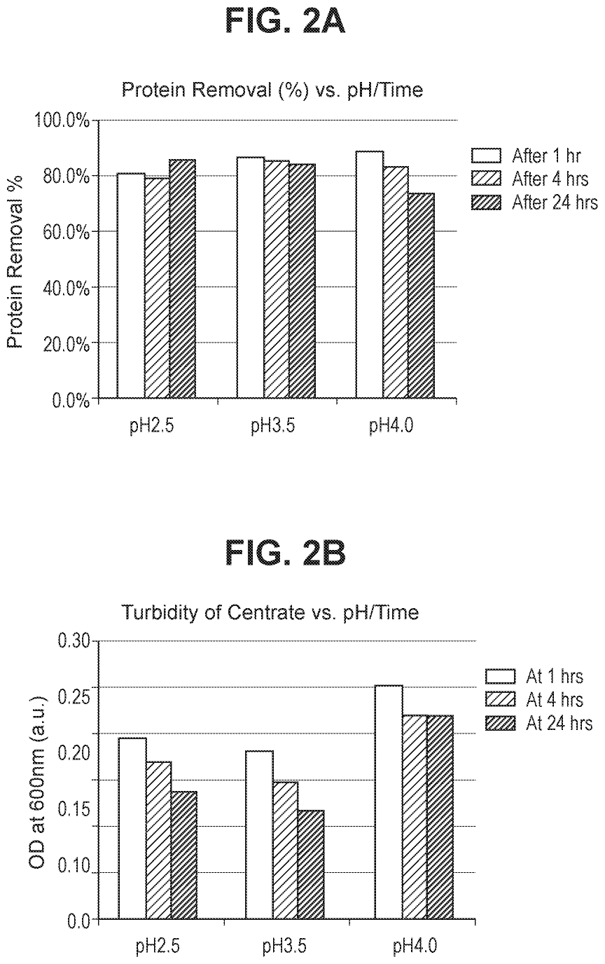

Then the pH was adjusted to the desired levels. The containers were then centrifuged after various hold time points at 12,000 g for 15 minutes. The supernatant was assay for protein, polysaccharide and clarity. The effect of pH on the protein removal and clarity in the presence of 2% alum at hold times of 1, 4 and 24 hours is shown in FIG. 2. This data shows that protein removal at pH 2.5-4.0 and 2% alum was quite effective. Over 80% of protein impurities were removed in this single step. The clarity of the centrate was affected by both pH and hold time. FIG. 2 shows that a pH of 3.5 gave the highest centrate clarity.

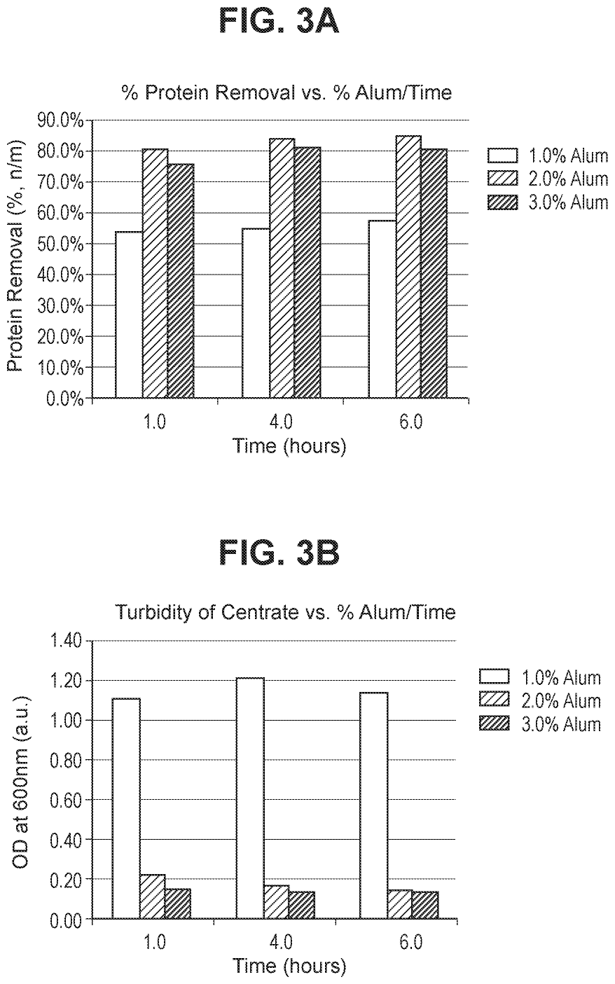

The effect of alum concentration and hold time on protein removal and centrate clarity at pH 3.5 is shown in FIG. 3. The hold time study was conducted at ambient temperature (20±2° C.). The results show that 1.0% alum was not sufficient at either protein removal or clarifying the centrate. The difference between 2% and 3% alum was not significant.

2.2 Effect of Temperature

The flocculated broth (pH 3.5 and 2% alum) was heated to 50° C., and held for 30 and 60 minutes. After cooling to ambient temperature, the samples were centrifuged at 12,000 g. The clarity of the centrate was measured compared to centrate from a flocculation performed at ambient temperature. The OD600 of the centrate from the ambient temperature flocculation was 0.99. After 30 minutes at 50° C., the OD600 decreases to 0.13 and after 60 minutes at 50° C. the OD600 is further reduced to 0.04. This clearly demonstrates that clarity of the centrate can be significantly improved by performing the flocculation at higher temperature.

2.3 Effect of Variables that Impact Flocculation

In order to better define the effect of variables that impact on the serotype 8 flocculation process, a study was conducted. We examined the factors of alum concentration, pH, temperature, and hold time on polysaccharide recovery, clarity and impurity removal.

The specified amount of alum was added to the broth at room temperature, and then the pH was adjusted with either 5N H₂SO₄ or 5N NaOH. Samples were placed in a water bath, which was set at the desired temperature, and at each time point, samples were taken analysis, and then were centrifuged at 12,000×g. The supernatant was analyzed for polysaccharide concentration, protein and turbidity (OD600)

| | Alum | pH | Temp | Hold time | Polysacc (% | Protein (% |
|---|---|---|---|---|---|---|
| Sample | (%) | | (° C.) | (hr) | recov) | removal) |
| 1 | 2 | 3 | 40 | 2 | 88 | 94 |
| 2 | 2 | 2 | 40 | 2 | 82 | 95 |
| 3 | 2 | 3 | 40 | 3 | 84 | 95 |
| 4 | 4 | 2 | 60 | 1 | 81 | 95 |
| 5 | 2 | 3 | 40 | 2 | 86 | 94 |
| 6 | 4 | 4 | 60 | 3 | 73 | 94 |
| 7 | 0 | 4 | 60 | 3 | 71 | 79 |
| 8 | 4 | 4 | 20 | 1 | 61 | 93 |
| 9 | 0 | 2 | 20 | 3 | 50 | 93 |
| 10 | 4 | 2 | 20 | 3 | 69 | 91 |
| 11 | 0 | 2 | 60 | 1 | 71 | 95 |
| 12 | 0 | 3 | 40 | 2 | 41 | 94 |
| 13 | 4 | 2 | 20 | 1 | 56 | 92 |
| 14 | 2 | 4 | 40 | 2 | 87 | 95 |
| 15 | 0 | 4 | 20 | 1 | 94 | 81 |
| 16 | 4 | 4 | 60 | 1 | 83 | 94 |
| 17 | 4 | 4 | 20 | 3 | 53 | 93 |
| 18 | 4 | 2 | 60 | 3 | 92 | 94 |
| 19 | 2 | 3 | 20 | 2 | 59 | 95 |
| 20 | 0 | 2 | 20 | 1 | 79 | 73 |
| 21 | 0 | 4 | 20 | 3 | 79 | 76 |
| 22 | 2 | 3 | 40 | 1 | 74 | 95 |
| 23 | 2 | 3 | 60 | 2 | 88 | 94 |
| 24 | 2 | 3 | 40 | 2 | 89 | 94 |
| 25 | 4 | 3 | 40 | 2 | 84 | 94 |
| 26 | 0 | 4 | 60 | 1 | 83 | 65 |
| 27 | 0 | 2 | 60 | 3 | 100 | 93 |

Analysis of the results showed the desirability for the pH, percent alum and hold time for the flocculation unit operation was fairly broad, pH: 2.75-3.75; Alum: 1.5-3.0% w/v; and hold time: 1.5-3 hours. The desired range for the temperature was around 45-60° C.

3. Centrifugation

Centrifugation has been conducted to clear centrate so that it can be filtered with reasonable capacity. The centrifugal speed was set at 12,000-xg.

4. Depth Filtration

Although centrifugation is the primary solid/liquid separation unit operation, it does not remove all of the particles from the feed stream, a depth filtration unit operation was incorporated between the centrifugation and the first ultrafiltation unit operation.

Initial studies were conducted using a filter which having a nominal retention range of 0.25-1.0 micron. The clarity of the centrate had impact on the filter capacity.

In particular where the flocculation was performed at about 20° C., the centrate clarity was not as good, with OD600 in the range of 0.8-1.4 and the filter capacity was affected. When using higher temperature flocculation conditions, the depth filtration process showed more robust and consistent capacity, with a filter capacity greater than 400 L/m2, even with OD600 of centrate ranging from 0.04 to 0.2.

5. Optional 0.45 Micron Filtration

Although optional, a 0.45 micron filter was used in some samples post depth filtration.

6. Ultrafiltration/Diafiltration-(UFDF-1)

Purification begins with the depth filtrate (from step 4 or 5 above).

This operation replaces the spent fermentation media with a buffer while reducing the levels of low molecular weight host cell impurities and residual floculant (aluminum).

Before the first UFDF, the depth filtrate was adjusted to 7.0 using 5N sodium hydroxide. Alternatively, the pH is not adjusted before UFDF diafiltration is conducted against sodium citrate/sodium phosphate, pH 7.0 (e.g. 10 mM phosphate/25 mM citrate pH 7.0) as diafiltration buffer.

7. Carbon Filtration

This unit operation reduces the level of host cell impurities such as proteins and nucleic acids as well as colored impurities (see WO2008118752).

The effectiveness of removing protein impurities by carbon filter was studied. Three 7″ diameter R32SP carbon filters in series. The retentate from UFDF-1 was filtered at a flow rate of 40 LMH and the UV280 for the carbon filtrate was recorded.

The UV280 signal for the retentate was only 460-mAU before carbon filtration, fairly low compared to the baseline of the water rinse (380-mAU). This suggested that most protein related impurities had already been removed by the previous unit operations. However, carbon filters still removed the remaining residual impurities quite effectively. This is shown in the reduction of UV280 signal after the filters were put in line, where the UV signal dropped to the baseline. This data indicated that protein related impurities were removed by a single pass through the carbon filters.

8. Ultrafiltration/Diafiltration-(UFDF-2)

This unit operation concentrates the product to the desired concentration and replaces the 25 mM sodium citrate, 10 mM sodium phosphate, pH 7.0 with the correct buffer for conjugation. This step is performed using a 30-kDa molecular weight cutoff filter.

The presence of residual citrate may interfer with the conjugation chemistry. Different solutions have been used as diafiltration buffer: a combination of 50 mM NaCl and water, or 25 mM potassium phosphate pH 6.0 as the diafiltration buffer.

The effect of 25 mM potassium phosphate pH 6.0 on citrate removal was evaluated. In this experiment, carbon filtrate was concentrated 2.6 folds and then diafiltered against 25 mM potassium phosphate pH 6.0. Samples were removed and analyzed for residual citrate at the various points of diafiltration.

A rejection coefficient of 0.13 was obtained. Less than seven diavolumes of 25 mM potassium phosphate pH 6.0 are required to reach a reduction of 6-logs.

9. Consistency

To demonstrate that the recovery and purification process described above could produce reproducible results, three consistency batches were manufactured. The entire fermentation batch was flocculated and centrifuged using the process described above.

The step and overall yields for the three consistency batches are shown in Table 2. All step yields are approximately 77-99%, very reproducible and robust.

TABLE 2
Step and Overall Yields for serotype 8 Consistency Batches

| | VRU Consistency Batches | | |
|---|---|---|---|
| Serotype 8 | 001 | 002 | 003 |
| Fermentation | NA | NA | NA |
| Broth | | | |
| Centrate | 100 | 100 | 100 |
| Depth Filtrate | 77 | 90 | 88 |
| UFDF1 Retentate | 102 | 102 | 100 |
| Carbon Filtrate | 95 | 93 | 92 |
| UFDF2 Retentate | 97 | 97 | 96 |
| Final Filtration | 99 | 99 | 99 |
| Overall Yield | 72 | 84 | 78 |

The analytical results for the three consistency batches are shown in Table 3. The three consistency batches met all of the pre-defined acceptance criteria.

TABLE 3
Analytical Results for serotype 8 Consistency Batches

| Serotype 8 | Consistency | | |
|---|---|---|---|
| Assay | 001 | 002 | 003 |
| Residual NLS | <LOQ | <LOQ | <LOQ |
| Residual | <0.16% | <0.15% | <0.14% |
| Nucleic Acids | | | |
| Residual Protein | 0.3% | 0.3% | 0.3% |
| Residual C poly | 2.5 wt % | 2.8 wt % | 2.4 wt % |

### Example 2. Purification of Pneumococcal Polysaccharide Serotype 33F

The process flow diagram for the purification of pneumococcal polysaccharide 33F is shown in FIG. 1. The process begins with NLS treated fermentation broth and includes recover unit operations (flocculation, centrifugation and depth filtration) followed by purification unit operations (ultrafiltration, and carbon filtration).

1. Starting Material

The process begins with NLS inactivated fermentation broth of S. pneumonia serotype 33F. Cultures were grown in Hy-Soy medium. At the end of growth (as indicated by no further increase in optical density), the cultures were treated with NLS (see EP2129693).

2. Flocculation

The main purpose of this step is to precipitates cell debris, host cell proteins and nucleic acids. It also aids in the downstream clarification unit operations. The flocculation has been performed using fermentation broth that has been lysed by the addition of NLS.

2.1 Effect of pH

Experiments were Conducted to Examine the Effect of pH.

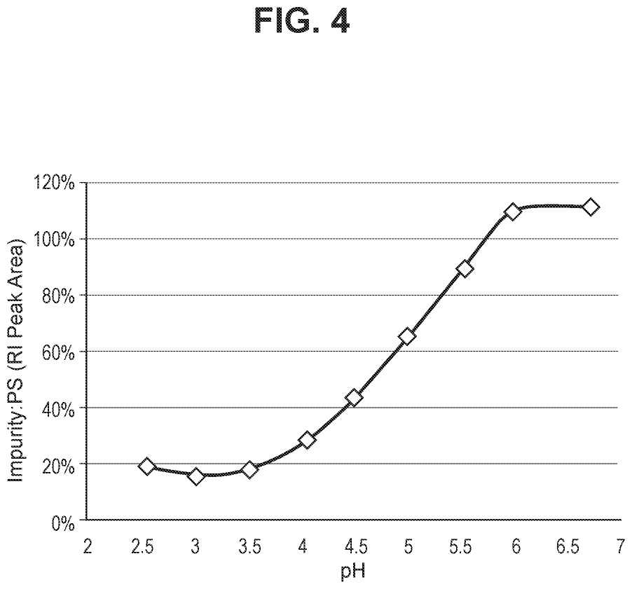

To determine the optimum pH for flocculation of serotype 33F, an acid titration study was performed on inactivated 33F fermentation broth. The results from that study are shown in FIG. 4. The graph of impurity removal versus pH shows that the maximum impurity removal is achieved at/below pH 3.5.

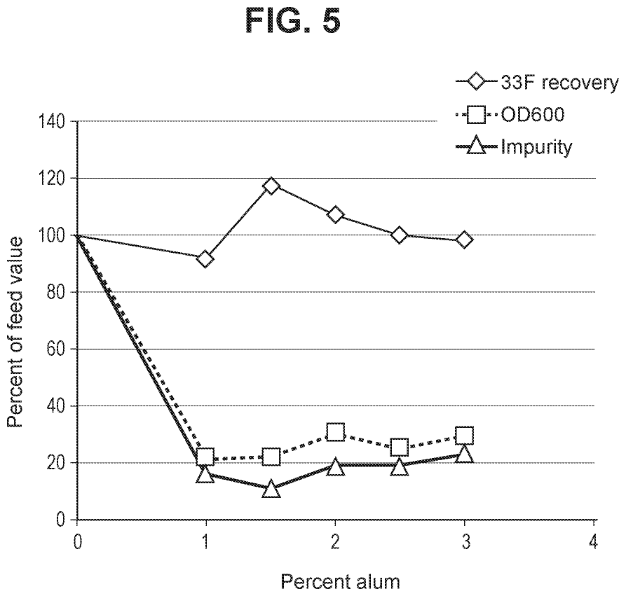

Using the pH identified in the previous experiment, an alum flocculation study was conducted. Results from that study are shown in FIG. 5. The sedimentation rate as measured by the OD600 is largely invariant at alum concentrations >1.0%. The highest impurity removal is at 1.5% alum, although there is not a significant difference seen between 1-3% alum. There was no significant change in polysaccharide concentration over the titration range.

2.2 Effect of Other Parameters

To determine the effect of rate of alum addition, a fermentation batch was split in two and the stock alum solution was added either over 3 min or over 60 min. There was no significant effect on either clarity post centrifugation or depth filter capacity. This indicates that alum addition rate is not a significant process parameter.

To further refine the flocculation conditions, a design of experiment (DOE) was set up that examined the effect of alum concentration, pH, temperature, and hold time on polysaccharide recovery, clarity and protein removal. The factors examined are shown in Table 4.

TABLE 4
DOE Factors Examined in 33F Flocculation Study

| Factor | Range |
|---|---|
| Alum concentration (% w/v) | 0-4 |
| pH | 2-5 |
| Temperature (° C.) | 20-60 |
| Hold time (min) | 30-90 |

Polysaccharide recovery was not significantly impacted under any of the conditions tested, as all conditions gave greater than 95% recovery. Similarly, protein removal was greater than 90% for all of the conditions tested. The concentration of alum has the greatest impact on clarity as measured by OD600. At low alum concentrations, the OD600 increased. There were also slight increases in the clarity as the temperature decreased and the pH increased.

To determine the effect flocculation conditions have on clarification unit operations, a continuous centrifugation study was conducted on fermentation broth that had been flocculated at 50° C.

As observed with the broth flocculated at 20° C., there was no significant increase in the centrate clarity at feed rates from 400-1200 mL/min for the broth flocculated at 50° C. However, both the centrate clarity and depth filter capacity increased significantly compared to the 20° C. flocculation. Using the new flocculation conditions, the depth filter capacity is greater than 400 L/m2.

To confirm that increasing the flocculation temperature from 20° C. to 50° C. improved the clarity of the centrate and thus the capacity of the depth filters (see below). The clarity of the centrate and depth filtrate were measured (see Table 5).

TABLE 5
Effect of Flocculation Temperature on
Centrate and Depth Filtrate Clarity

| Flocculation temerature | Centrate OD600 | Depth Filtrate OD600 |
|---|---|---|
| 20° C. | 0.060 | 0.025 |
| 50° C. | 0.031 | 0.016 |

3. Centrifugation

Centrifugation has been conducted to clear centrate so that it can be filtered with reasonable capacity. The centrifugal speed was set at 12,000-xg.

4. Depth Filtration

Although centrifugation is the primary solid/liquid separation unit operation, it does not remove all of the particles from the feed stream, a depth filtration unit operation was incorporated between the centrifugation and the first ultrafiltation unit operation.

5. Optional 0.45 Micron Filtration

Although optional, a 0.45 micron filter was used in some samples post depth filtration.

6. Ultrafiltration/Diafiltration-(UFDF-1)

Purification begins with the depth filtrate (from step 4 or 5 above).

This operation replaces the spent fermentation media with a buffer while reducing the levels of low molecular weight host cell impurities and residual floculant (aluminum).

In initial experiments the pH of the depth filtrate was adjusted from 3.5 to 7.0 using 5N sodium hydroxide. Although this did not affect the molecular weight of the polysaccharide, it did result in partial de-acetylation of the O-acetyl groups on 33F.

It has been proposed that the deacetylation was due to high local pH that resulted during the neutralization with 5N sodium hydroxide. Therefore it was decided to adjust the pH of the 33F solution during the diafiltration after concentrating the depth filtrate to a manageable volume.

The diafiltration was performed against sodium citrate/sodium phosphate, pH 7.0 (e.g. 10 mM phosphate/25 mM citrate pH 7.0).

Alternatively, 25 mM EDTA could also be used instead of citrate.

7. Carbon Filtration

This unit operation reduces the level of host cell impurities such as proteins and nucleic acids as well as colored impurities (see WO2008118752).

The carbon filtration step can be performed as either a single pass mode or in a recirculation mode. To determine which mode of operation works best for serotype 33F, a recirculation carbon filtration study was performed. In this experiment retentate from UFDF-1 was filtered through two through 47 mm Cuno R32SP discs in series (35 cm2 total area) at 170LMH and the impurity level was determined after each cycle (total of 5 cycles). The impurity removal was best at the lowest feed challenge of ˜30 L/m2. Additional impurity removal at greater than one cycle was insignificant indicating there is little or no benefic to using the recirculation mode.

8. Optional 0.2 Micron Filtration

Although optional, a 0.2 micron filter was used in some samples carbon filtration.

9. Ultrafiltration/Diafiltration-(UFDF-2)

This unit operation concentrates the product to the desired concentration and replaces the 25 mM sodium citrate, 10 mM sodium phosphate, pH 7.0 with the correct buffer for conjugation. This step is performed using a 30-kDa molecular weight cutoff filter.

The presence of residual citrate may interfere with the conjugation chemistry. To be highly reduced the level of citrate, diafiltration experiments were performed using different buffers.

In these experiments, carbon filtrate was concentrated 4-fold and then diafiltered against different buffers. Samples were removed and analyzed for residual citrate.

Water had the highest rejection coefficient of 50% and would have required approximately 22 diavolumes to reach the target reduction.

Both 10 mM sodium phosphate pH 7.0 and 10 mM potassium phosphate pH 6.5 had similar rejection coefficients of approximately 20% and would require 10 diavolumes to reach the target reduction of 6-logs.

Sodium chloride at 25 mM had the lowest rejection coefficient of 8% and would reach the target reduction in 7 diavolumes. Reducing the sodium chloride concentration to 10 mM resulted in an increase of the rejection coefficient to 28%.

To ensure that residual citrate level was achieved, the concentration of sodium chloride was increased to 50 mM. After six diavolumes of 50 mM sodium chloride, the retentate was diafiltered against water for six more diavolumes.

10. Sterile Filtration

The final unit operation prior to filling into storage bottles is a sterile filtration (0.2 micron filtration).

11. Consistency

To demonstrate that the recovery and purification process described above could produce reproducible results, three consistency batches were manufactured. The fermentation batch was flocculated and centrifuged using the process described above. The step and overall yields for the three consistency batches are shown in Table 6. All step yields are approximately 90% or higher and very reproducible. The overall yield average is 73%.

TABLE 6
Step and Overall Yields for serotype 33F Consistency Batches

| | Consistency Batches | | |
|---|---|---|---|
| Serotype 33F | 001 | 002 | 003 |
| Fermentation | NA | NA | NA |
| Broth | | | |
| Centrate | 100 | 100 | 100 |
| Depth Filtrate | 90 | 90 | 91 |
| UFDF1 Retentate | 101 | 102 | 99 |
| Carbon Filtrate | 87 | 86 | 87 |
| UFDF2 Retentate | 92 | 95 | 93 |
| Final Filtration | 99 | 99 | 99 |
| Overall Yield | 72 | 74 | 72 |

The analytical results for the three consistency batches are shown in Table 3. The three consistency batches met all of the pre-defined acceptance criteria.

The analytical results for the three consistency batches are shown in Table 7.

TABLE 7
Analytical Results for 33F Consistency
Batches (floculation at 20° C.)

| | | | |
|---|---|---|---|
| Assay | 001 | 002 | 003 |

| | | | |
|---|---|---|---|
| O-Acetylation | 0.87 | 0.85 | 0.90 |
| Residual Citrate | <LOQ | <LOQ | <LOQ |
| Residual NLS | <LOQ | <LOQ | <LOQ |
| Residual Nucleic Acids | <0.02% | <0.02% | <0.04% |
| Residual Protein | 0.2% | 0.2% | 0.3% |

| | | | | | | |
|---|---|---|---|---|---|---|
| Residual C poly | 4.4 | wt % | 4.2 | wt % | 4.7 | wt % |
| Residual Al | <1 | ppm | <1 | ppm | <1 | ppm |

Two more batches were manufactured using the same procedure except that the flocculation was performed at 50° C. instead of 20° C. The analytical results from these batches along with the average results from the consistency batches are shown in Table 8. These results clearly show that increasing the flocculation temperature did not have any impact on product quality.

TABLE 8
Analytical Results for 33F floculation at 50° C.

| Assay | 50° C.-1 | 50° C.-2 |
|---|---|---|
| O-Acetylation | 1.03 | 1.04 |
| Residual Citrate | <LOQ | <LOQ |
| Residual NLS | <LOQ | <LOQ |
| Residual Nucleic Acids | 0.02% | 0.02% |
| Residual Protein | 0.2% | 0.2% |
| Residual C poly | 4.1 | 3.2 |
| Overall Yield | 68% | 57% |

### Example 3. Purification of Pneumococcal Polysaccharide Serotype 15B

The process flow diagram for the purification of pneumococcal polysaccharide 15B is shown in FIG. 1. The process begins with NLS treated fermentation broth and includes recover unit operations (flocculation, centrifugation and depth filtration) followed by purification unit operations (ultrafiltration, and carbon filtration).

1. Starting Material The process begins with NLS inactivated fermentation broth of S. pneumonia serotype 15B. Cultures were grown in Hy-Soy medium. At the end of growth (as indicated by no further increase in optical density), the cultures were inactivated with NLS (see EP2129693).

2. Flocculation

The main purpose of this step is to precipitates cell debris, host cell proteins and nucleic acids. It also aids in the downstream clarification unit operations. The flocculation has been performed using fermentation broth that has been lysed by the addition of NLS.

Similarly to Example 1 and 2, experiments were conducted to examine the effect of different parameters on the flocculation.

The effect of variables were pH, percent alum and hold time.

This data showed that protein removal at pH 2.5-4.0 and 1-3% alum was quite effective with over 90% of protein impurities removed in this a single step. This efficiency of impurity removal was not affected by the hold time (1, 4 or 24 hours).

To confirm the flocculation conditions developed for 15B, a DOE study was performed to examine the effect of alum concentration, pH and hold time on polysaccharide recovery, clarity and impurity removal. The range of factors for pH, alum percent and hold time are: 2-4, 0-4% w/v, and 1-4 hours, respectively.

The experimental data for this DOE study is shown in Table 9. A total of 20 experiments were performed within the design space.

TABLE 9

| | Alum | | Hold time | Polysacc | Protein |
|---|---|---|---|---|---|
| Sample | (%) | pH | (hr) | (% recov) | (% removal) |
| 1 | 2 | 3 | 2 | 93 | 93 |
| 2 | 2 | 4 | 2 | 97 | 91 |
| 3 | 4 | 2 | 3 | 92 | 92 |
| 4 | 4 | 4 | 3 | 92 | 89 |
| 5 | 2 | 3 | 3 | 92 | 93 |
| 6 | 2 | 3 | 2 | 93 | 93 |
| 7 | 0 | 3 | 2 | 89 | 94 |
| 8 | 2 | 3 | 1 | 89 | 93 |
| 9 | 4 | 3 | 2 | 90 | 92 |
| 10 | 2 | 3 | 2 | 93 | 93 |
| 11 | 2 | 3 | 2 | 93 | 93 |
| 12 | 0 | 4 | 1 | 97 | 90 |
| 13 | 0 | 2 | 1 | 92 | 94 |
| 14 | 2 | 3 | 2 | 93 | 93 |
| 15 | 4 | 4 | 1 | 89 | 88 |
| 16 | 2 | 3 | 2 | 93 | 93 |
| 17 | 0 | 2 | 3 | 92 | 94 |
| 18 | 2 | 2 | 2 | 92 | 92 |
| 19 | 0 | 4 | 3 | 96 | 91 |
| 20 | 4 | 2 | 1 | 90 | 92 |

Results suggested that within the design space, the desirability for the pH, percent alum and hold time for the flocculation unit operation was fairly broad, pH: 2.7-3.8; alum: 1-2.5% (w/v); and hold time: 1.5-3 hours. Similar results were observed in the DOE studies of other serotypes.

The above experiments were conducted at 20° C.

To further determine the effect flocculation conditions have on clarification unit operations, a continuous centrifugation study was conducted on fermentation broth that had been flocculated at 50° C.

As observed with the broth flocculated at 20° C., there was no significant increase in the centrate clarity at feed rates from 400-800 mL/min for the broth flocculated at 50° C.

3. Centrifugation

Centrifugation has been conducted to clear centrate so that it can be filtered with reasonable capacity. The centrifugal speed was set at 12,000-xg.

4. Depth Filtration

Although centrifugation is the primary solid/liquid separation unit operation, it does not remove all of the particles from the feed stream, a depth filtration unit operation was incorporated between the centrifugation and the first ultrafiltation unit operation.

5. Optional 0.45 Micron Filtration

Although optional, a 0.45 micron filter was used in some samples post depth filtration.

6. Ultrafiltration/Diafiltration-(UFDF-1)

Purification begins with the depth filtrate (from step 4 or 5 above).

This operation replaces the spent fermentation media with a buffer while reducing the levels of low molecular weight host cell impurities and residual floculant (aluminum).

In initial experiments the pH of the depth filtrate was adjusted from 3.5 to 7.0 using 5N sodium hydroxide. However, this can result in partial de-acetylation of the O-acetyl groups on 15B.

Therefore it was decided to adjust the pH of the 15B solution during the diafiltration after concentrating the depth filtrate to a manageable volume.

In the buffer selection studies for 15B, the citrate concentrations between 10-50 mM have been tested with different concentrations of sodium phosphate.

The diafiltration was performed against sodium citrate/sodium phosphate, pH 7.0 (e.g.

10 mM phosphate/25 mM citrate pH 7.0).

7. Carbon Filtration

This unit operation reduces the level of host cell impurities such as proteins and nucleic acids as well as colored impurities (see WO2008118752).

Three 7″ diameter Cuno R32SP carbon filters were used in series. The retentate from UFDF-1 was first by pass the filter so that the UV signal at 280 nm was recorded for the starting solution. After that the retentate was filtered at flow rate of 32 LMH and the UV280 for the carbon filtrate was recorded and compared with that of the retentate. A reduction of about 95% in UV280 signal demonstrated that protein related impurities are removed by the single pass through the carbon filters.

The carbon filtration step can be performed as either a single pass mode or multiple or in a recirculation mode. To determine if the addition pass through carbon would add any benefit for serotype 15B, an experiment was carried out where the retentate from UFDF-1 was filtered through three 7-inch Cuno R32SP discs in series at 64 LMH. The impurity level, UV280 level and Borate Lowry assay for protein were determined after each pass. Results showed that a single pass was sufficient to remove most of the impurities. The protein concentration for the single pass filtrate and the second pass filtrate were 25.2 and 20.6 μg/mL, respectively. When factoring the dilution due to rinsing the filters, amount of protein in the first pass filtrate and in the second pass filtrate was about the same.

8. Ultrafiltration/Diafiltration-(UFDF-2)

This unit operation concentrates the product to the desired concentration and replaces the 25 mM sodium citrate, 10 mM sodium phosphate, pH 7.0 with the correct buffer for conjugation. This step is performed using a 30-kDa molecular weight cutoff filter.

The presence of residual citrate may interfere with the conjugation chemistry. To be highly reduced the level of citrate, diafiltration experiments were performed using different buffers (see examples 1 and 2).

9. Homogenization

Purified 15B polysaccharides can be homogenized, for example mechanically sized (see e.g. WO2015110942).

10. Sterile Filtration

The final unit operation prior to filling into storage bottles is a sterile filtration (0.2 micron filtration).

11. Consistency

To demonstrate that the recovery and purification process described above could produce reproducible results, three consistency batches were manufactured. The fermentation batch was flocculated and centrifuged using the process described above.

The step and overall yields for the three consistency batches are shown in Table 10. All step yields are approximately 75-98%, very reproducible and robust. The overall yield average is 60%.

TABLE 10
Step and Overall Yields for serotype 15B Consistency Batches

| | Consistency Batches | | |
|---|---|---|---|
| Serotype 15B | 001 | 002 | 003 |
| Fermentation | NA | NA | NA |
| Broth | | | |
| Centrate | 100 | 100 | 100 |
| Depth Filtrate | 98 | 88 | 83 |
| UFDF1 Retentate | 75 | 86 | 85 |
| Carbon Filtrate | 90 | 88 | 88 |
| UFDF2 Retentate | 93 | 96 | 97 |
| Homogenization | 90 | 92 | 95 |
| Overall Yield | 55 | 66 | 57 |

The analytical results for the three consistency batches are shown in Table 11. The three consistency batches met all of the pre-defined acceptance criteria.

The analytical results for the three consistency batches are shown in Table 11.

TABLE 11
Analytical Results for 15B Consistency Batches

| Test | 15B-001 | 15B-002 | 15B-003 |
|---|---|---|---|
| O-Acetylation | 0.94 | 0.88 | 0.87 |
| Glycerol | 0.97 | 0.92 | 0.93 |
| Residual Citrate | <LOQ | <LOQ | <LOQ |
| Residual NLS | <LOQ | <LOQ | <LOQ |
| Residual Nucleic | <0.12 | <0.13 | <0.13 |
| Acids | | | |
| Residual Protein | 0.2% | 0.2% | 0.2% |
| Residual C poly | 1.5% | 1.8% | 1.8% |
| Residual Al | <0.05 ppm | <0.05 ppm | <0.05 ppm |

Two more batches were manufactured using the same procedure except that the flocculation was performed at 50° C. instead of 20° C. The analytical results from these batches along with the average results from the consistency batches are shown in Table 8. These results clearly show that increasing the flocculation temperature did not have any impact on product quality.

TABLE 12
Comparison of 50° C. Batches

| Assay | 50° C.-1 | 50° C.-2 |
|---|---|---|
| O-Acetylation | 0.94 | 0.94 |
| Residual Citrate | <LOQ | <LOQ |
| Residual NLS | <LOQ | <LOQ |
| Residual Nucleic Acids | <0.02 | N/A |
| Residual Protein | N/A | N/A |
| Residual C poly | 1.0% | 0.9% |
| Overall Yield | 43% | N/A |

### Example 4. Purification of Pneumococcal Polysaccharide Serotype 22F

The process flow diagram for the purification of pneumococcal polysaccharide 22F is shown in FIG. 1. The process begins with NLS inactivated fermentation broth and includes recover unit operations (flocculation, centrifugation and depth filtration) followed by purification unit operations (ultrafiltration, and carbon filtration).

1. Starting Material The process begins with NLS inactivated fermentation broth of S. pneumonia serotype 22F. Cultures were grown in Hy-Soy medium. At the end of growth (as indicated by no further increase in optical density), the cultures were treated with NLS. (see EP2129693).

2. Flocculation

The main purpose of this step is to precipitates cell debris, host cell proteins and nucleic acids. It also aids in the downstream clarification unit operations. The flocculation has been performed using fermentation broth that has been lysed by the addition of NLS. 2.1 Effect of pH and Alum

Similarly to Example 1 and 2, experiments were conducted to examine the effect of different parameters on the flocculation.

This data showed that protein removal at pH 2.5-4.0 and 1.5-3% alum was quite effective with over 90% of protein impurities removed in this a single step.

To confirm the flocculation conditions developed for 22F, a DOE study was performed to examine the effect of alum concentration and pH on polysaccharide recovery, clarity and impurity removal.

Protein removal efficiency was very high at reduced pH. Combined alum and pH was effective.

2.2 Effect of Temperature

A study was conducted to examine the effect of temperature on flocculated broth particle size. After flocculation (2% w/v alum, pH 3.5) at 20° C., the flocculated broth was heated to desired temperature and held for one hour. After cooling to ambient (15-25° C.), the flocculated broth was centrifuged at 12,000-xg and the clarity (OD600) a was determined.

Results are shown in Table 13.

TABLE 13
Results of flocculated particle size and
centrate OD at various temperatures

| Post Flocculation 1 hr | Flocculated broth | Centrate |
|---|---|---|
| Hold temp, ° C. | particle size, μm | OD600 |
| Broth | 10.3 | NA |
| 20° C. | 178 | 0.066 |
| 30° C. | 206 | 0.033 |
| 40° C. | 210 | 0.028 |
| 50° C. | 203 | 0.027 |

There was a significant decrease in the centrate OD600 at increased temperatures.

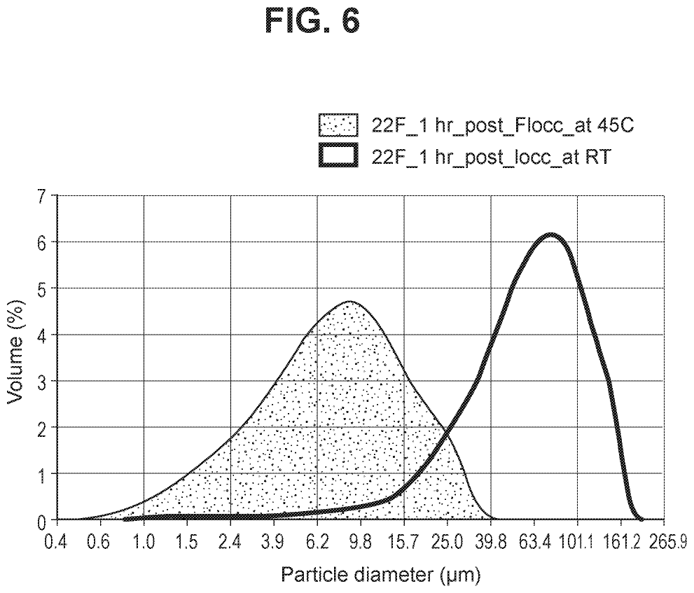

In previous experiments it was observed that the flocculated broth particle size was larger at higher temperatures. A visual comparison showing the change in particle size with flocculation temperature is shown in FIG. 6. For the first experiment the flocculation temperature was held room temperature (RT) and for the second experiment it was increased to 45° C. for 1 hr.

In FIG. 6 the flocculated broth mean particle size after 1 hr hold at room temperature and 45° C. heated were 9.8 μm and 65 μm respectively. There was a significant increase in particle size for the flocculated broth heated to 45° C. In addition to larger particle size, there is also a reduction in the amount of fine particles (<1 μm). The formation of large particles and reduction of fine particles helps with the further steps (e.g. centrifugation and depth filtration) and results in a clearer centrate.

Two additional 22F batches were manufactured where the only change was the flocculation temperature (20° C. or 50° C.). The clarity of the centrate (post centrifugation) and post depth filtration (see below) from these batches are shown in Table 14.

TABLE 14
Effect of Flocculation Temperature on
Centrate and Depth Filtrate Clarity

| Batch | Centrate OD600 | Depth Filtrate OD600 |
|---|---|---|
| 1 (20° C.) | 0.09 | 0.024 |
| 2 (50° C.) | 0.04 | 0.016 |

3. Centrifugation

Centrifugation has been conducted to clear centrate so that it can be filtered with reasonable capacity. The centrifugal speed was set at 12,000-xg.

4. Depth Filtration

Although centrifugation is the primary solid/liquid separation unit operation, it does not remove all of the particles from the feed stream, a depth filtration unit operation was incorporated between the centrifugation and the first ultrafiltation unit operation.

5. Optional 0.45 Micron Filtration

Although optional, a 0.45 micron filter was used in some samples post depth filtration.

6. Ultrafiltration/Diafiltration-(UFDF-1)

Purification begins with the depth filtrate (from step 4 or 5 above).

This operation replaces the spent fermentation media with a buffer while reducing the levels of low molecular weight host cell impurities and residual floculant (aluminum).

Prior to performing the carbon filtration, the pH of the 22F polysaccharide was adjusted to 7.0±0.5. As shown above, use of NaOH to adjust pH can result in partial de-acetylation of the O-acetyl groups. Since 22F polysaccharide also contains an O-acetyl group, it was decided to adjust the pH of the 22F solution during the diafiltration after concentrating the depth filtrate to a manageable volume.

In the buffer selection studies for serotype 22F, citrate concentrations between 0-40 mM were tested with different concentrations of sodium phosphate.

The highest diafiltration fluxes are obtained when the citrate concentration greater than 20 mM and the phosphate concentration is 10 mM or less.

7. Carbon Filtration

This unit operation reduces the level of host cell impurities such as proteins and nucleic acids as well as colored impurities (see WO2008118752).

The UFDF1 retentate was filtered through three or four 7″ diameter R32SP carbon filters stacked in series. A series of experiments were performed to measure the effectiveness of R32SP carbon filters in removing residual UV and RI impurities from the UFDF1 retentate. There was at least 95% reduction in the UV 260/280 nm absorbance. This indicates significant removal of protein and nucleic acid related impurities from the UFDF1 retentate. Results show that carbon has excellent capacity for the protein related impurity removal.

8. Ultrafiltration/Diafiltration-(UFDF-2)

This unit operation concentrates the product to the desired concentration and replaces the 25 mM sodium citrate, 10 mM sodium phosphate, pH 7.0 with the correct buffer for conjugation. This step is performed using a 30-kDa molecular weight cutoff filter.

The presence of residual citrate may interfere with the conjugation chemistry. To be highly reduced the level of citrate, diafiltration experiments were performed using different buffers (see examples 1 and 2).

9. Homogenization

Purified 22F polysaccharides can be homogenized, for example mechanically sized (see e.g. WO2015110942).

10. Sterile Filtration

The final unit operation prior to filling into storage bottles is a sterile filtration (0.2 micron filtration).

11. Consistency

To demonstrate that the recovery and purification process described above could produce reproducible results, three consistency batches were manufactured (flocculation temperature 20° C.). The fermentation batch was flocculated and centrifuged using the process described above.

The step and overall yields for the three consistency batches are shown in Table 14. All step yields are approximately 90% or higher and very reproducible. The overall yield average is 58%.

TABLE 14
Step and Overall Yields for 22F polysaccharide
Consistency Batches

| Unit Operation | 01 | 02 | 03 |
|---|---|---|---|
| Fermentation Broth | NA | NA | NA |
| Centrate | 100 | 100 | 100 |
| Depth Filtrate | 73 | 80 | 81 |
| UFDF1 Retentate | 98 | 93 | 85 |
| Carbon Filtrate | 86 | 87 | 82 |
| UFDF2 Retentate | 96 | 92 | 99 |
| Homogenization | NA¹ | 92 | NA¹ |
| Overall Yield | 59 | 55 | 56 |

¹01 and 03 were not homogenized

The analytical results for the three consistency batches are shown in Table 15.

TABLE 15
Analytical Results for 22F Consistency Batches

| | | | |
|---|---|---|---|
| Assay | 001 | 002 | 003 |

| | | | |
|---|---|---|---|
| O-Acetylation | 0.91/0.94 | 1.04/0.94 | 1.09/0.89 |
| native/sized | | | |
| Residual Citrate | <LOQ | <LOQ | <LOQ |
| Residual NLS | <LOQ | <LOQ | <LOQ |
| Residual | <0.02% | <0.02% | <0.02% |
| Nucleic Acids | | | |
| Residual | 0.2% | 0.2% | 0.2% |
| Protein | | | |

| | | | | | | |
|---|---|---|---|---|---|---|
| Residual C poly | 2.3 | wt % | 2.8 | wt % | 1.9 | wt % |
| Residual Al | <1 | ppm | <1 | ppm | <1 | ppm |

Two more batches were manufactured using the same procedure except that the flocculation was performed at 50° C. instead of 20° C. The analytical results from these batches along with the average results from the consistency batches are shown in Table 16. These results clearly show that increasing the flocculation temperature did not have any impact on product quality.

TABLE 16
Comparison of 50° C. Demo and Consistency Batches

| | 22F consistency | | |
|---|---|---|---|
| Assay | Batches(Average) | 50° C.-1 | 50° C.-2 |
| O-Acetylation | 1.01 | 1.08 | 1.07 |
| Residual Citrate | <LOQ | <LOQ | <LOQ |
| Residual NLS | <LOQ | NA¹ | NA¹ |
| Residual | <0.02% | 0.01% | 0.01% |
| Nucleic Acids | | | |
| Residual protein | 0.2% | 0.2% | 0.2% |
| Residual C poly | 2.3 | 2.5 | 1.8 |
| Overall Yield | 58% | 70.6% | 71.2 |

¹the batches were not analyzed for residual NLS

### Example 5. Purification of Pneumococcal Polysaccharide Serotype 10A

The process flow diagram for the purification of pneumococcal polysaccharide 10A is shown in FIG. 1. The process begins with NLS inactivated fermentation broth and includes recover unit operations (flocculation, centrifugation and depth filtration) followed by purification unit operations (ultrafiltration, and carbon filtration).

1. Starting Material

The process begins with NLS inactivated fermentation broth of S. pneumonia serotype 10A. Cultures were grown in Hy-Soy medium. At the end of growth (as indicated by no further increase in optical density), the cultures were inactivated with NLS (see EP2129693).

2. Flocculation

The main purpose of this step is to precipitates cell debris, host cell proteins and nucleic acids. It also aids in the downstream clarification unit operations. The flocculation has been performed using fermentation broth that has been lysed by the addition of NLS.

The optimum pH for flocculation of serotype 10A was determined by a DOE (see e.g. Examples 1 and 3).

Based on the Prediction Profiler the desirability is almost at optimum at pH 3.5 and 2% alum. It also shows that pH at slightly lower than 3.5 will help with minor improvements in protein removal and centrate clarity with a slight decrease in polysaccharide yield but at the same time peak purity might be a little higher.

Development work on other serotypes showed that increasing the temperature during flocculation resulted in higher depth filter capacity. However, heating of the broth can have an effect on the polysaccharide molecular weight. Experiments were carried out on 10A to determine if elevated temperatures would result in a decrease in molecular weight. In the first experiment, flocculated broths were incubated at temperatures of 20, 50, 60, and 70° C. and hold times of 1, 4 and 22 hours. Only samples from the 4 hour hold time at each temperature were purified. All of samples showed the same 1H-NMR spectra however the 60° C. and 70° C. samples showed significant reduction in molecular weight. In a second experiment, flocculated broths were incubated at temperatures of 20, 35, 45, and 55° C. with hold times of 1, 2, and 4 hours, and all of the samples were purified. This study showed very minor changes in molecular weight for up to 4 hours at 45° C. There was a slight decrease in the molecular weight observed in the first hour exposure at 55° C., but the difference was within the error of the assay.

3. Centrifugation

Centrifugation has been conducted to clear centrate so that it can be filtered with reasonable capacity. The centrifugal speed was set at 12,000-xg.

4. Depth Filtration

Although centrifugation is the primary solid/liquid separation unit operation, it does not remove all of the particles from the feed stream, a depth filtration unit operation was incorporated between the centrifugation and the first ultrafiltation unit operation.

5. Optional 0.45 Micron Filtration

Although optional, a 0.45 micron filter was used in some samples post depth filtration.

6. Ultrafiltration/Diafiltration-(UFDF-1)

Purification begins with the depth filtrate (from step 4 or 5 above).

This operation replaces the spent fermentation media with a buffer while reducing the levels of low molecular weight host cell impurities and residual floculant (aluminum).

Prior to performing the carbon filtration, the pH of the 22F polysaccharide was adjusted to 7.0±0.5.

Initially the diafiltration was performed against 10 mM sodium phosphate pH 7.0. This was successful in adjusting the pH to the desired value. However, during diafiltrations where only sodium phosphate was used, a white precipitate formed. The white solid was isolated and determined to contain aluminum phosphate. Aluminum phosphate is insoluble in water at neutral pH and is formed due to residual aluminum present after the flocculation step. To prevent formation of aluminum phosphate, it was decided to add a chelating agent to the diafiltration buffer. Based on work on other serotypes sodium citrate was selected. Citrate concentrations greater than 10 mM were effective at preventing haze formation over time. Citrate concentrations of 25 mM have been shown to remove residual aluminum to less than 1 ppm.

7. Carbon Filtration

This unit operation reduces the level of host cell impurities such as proteins and nucleic acids as well as colored impurities (see WO2008118752).

The UFDF1 retentate was filtered through a R32SP disc carbon filter. Results show that carbon has excellent capacity for the protein related impurity removal.

8. Ultrafiltration/Diafiltration-(UFDF-2)

This unit operation concentrates the product to the desired concentration and replaces the 25 mM sodium citrate, 10 mM sodium phosphate, pH 7.0 with the correct buffer for conjugation. This step is performed using a 30-kDa molecular weight cutoff filter.

The presence of residual citrate may interfere with the conjugation chemistry. To be highly reduced the level of citrate, diafiltration experiments were performed using different buffers (see examples 1 and 2).

9. Sterile Filtration

The final unit operation prior to filling into storage bottles is a sterile filtration (0.2 micron filtration).

10. Consistency

To demonstrate that the recovery and purification process described above could produce reproducible results, three consistency batches were manufactured (flocculation temperature 45° C.). The fermentation batch was flocculated and centrifuged using the process described above.

The step and overall yields for the three consistency batches are shown in Table 17. All step yields are greater than 72% and very reproducible. The overall yield average is 68%.

TABLE 17
Step and Overall Yields for 10A Consistency Batches

| Unit Operation | 10A-001 | 10A-002 | 10A-003 |
|---|---|---|---|
| Fermentation | NA | NA | NA |
| Broth | | | |
| Centrate | 100 | 100 | 100 |
| Depth Filtrate | 100 | 100 | 99 |
| UFDF1 | 91 | 93 | 97 |
| Retentate | | | |
| Carbon | 80 | 76 | 72 |
| Filtrate | | | |
| UFDF2 | 98 | 89 | 99 |
| Retentate | | | |
| Final Filtration | 99 | 99 | 99 |
| Overall Yield | 71 | 63 | 69 |

The analytical results for the three consistency batches are shown in Table 18.

TABLE 18
Analytical Results for 10A Consistency Batches

| | | | |
|---|---|---|---|
| Assay | 10A-001 | 10A-002 | 10A-003 |

| | | | |
|---|---|---|---|
| Residual Citrate | <LOQ | <LOQ | <LOQ |
| Residual NLS | <LOQ | <LOQ | <LOQ |
| Residual Nucleic | <0.15% | <0.11% | <0.21% |
| Acids | | | |
| Residual Protein | 0.5% | 0.4% | 0.4% |

| | | | | | | |
|---|---|---|---|---|---|---|
| Residual C poly | 5.0 | wt % | 4.9 | wt % | 4.4 | wt % |
| Residual Al | <0.05 | ppm | <0.05 | ppm | <0.05 | ppm |

### Example 6. Purification of Pneumococcal Polysaccharide Serotype 11A

The process flow diagram for the purification of pneumococcal polysaccharide 11A is shown in FIG. 1. The process begins with NLS inactivated fermentation broth and includes recover unit operations (flocculation, centrifugation and depth filtration) followed by purification unit operations (ultrafiltration, and carbon filtration).

1. Starting Material

The process begins with NLS inactivated fermentation broth of S. pneumonia serotype 11A. Cultures were grown in Hy-Soy medium. At the end of growth (as indicated by no further increase in optical density), the cultures were inactivated with NLS (see EP2129693).

2. Flocculation

The main purpose of this step is to precipitates cell debris, host cell proteins and nucleic acids. It also aids in the downstream clarification unit operations. The flocculation has been performed using fermentation broth that has been lysed by the addition of NLS.

Previous work on the purification process for other serotypes has shown that the clarification unit operation was impacted by the solution pH and the concentration of added alum. In order to understand the impact of these factors on the flocculation of serotype 11A, a DOE study was conducted, which also includes another factor—flocculation time, which may have impact on the flocculation process.

The ranges for each parameter are listed in Table 19.

TABLE 19
Parameter Range for Serotype 11A Flocculation DOE Study

| Range | Low | High |
|---|---|---|
| pH | 2 | 5 |
| Alum Concentration | 0% | 4% |
| Time | 15 min | 105 min |

A series of 20 experiments varying these 3 parameters were conducted.

The relationship between each variable (alum concentration, pH and flocculation time) and the responses (PS recovery, OD600 and protein removal) during the flocculation process has been analyzed by prediction profilers. The least important variable is flocculation time, which has almost no impact on either protein removal or clarity, and a very minor impact on polysaccharide recovery, when the alum concentration and pH are set at their middle points. Alum concentration mainly impacts the clarity. The optimal alum concentration is around 2.5%, which resulted in the best clarity. Although the OD600 difference is small when the alum concentration is between 1.5% to 3%. Flocculation pH on the other hand mainly impacts protein removal, with higher protein removal at lower pH. At pH 3.5 and below, the maximum impurity removal is achieved.

Additional experiments were designed to determine if increasing the flocculation temperature even improved downstream operations.

To determine the effect of temperature on flocculation of serotype 11A, an experiment was conducted at three different temperatures. In addition to temperature, two different agitation rates during flocculation were examined. 11A fermentation broth was flocculated using 2% alum, pH 3.5 at various temperatures for one hour. After flocculation, the broth was centrifuged and the clarity of the centrate was measured. The centrate was then filtered through a depth filter and the filter capacity was determined. The experimental conditions and results are shown in Table 20. The depth filter filtrate was further filtered through a 0.45 um dead-end filter using a Vmax model.

There was no difference in depth filter capacity for all four conditions as all were greater than 400 L/m². However, there was a significant difference in the 0.45 micron Vmax results. The flocculation at 50° C. had a Vmax of ˜1300 L/m² which was approximately 8-fold higher than the other conditions. Similarly, the flocculation at 10° C. had the lowest Vmax.

TABLE 20
Serotype 11A Flocculation with Different
Temperatures and Stirring Speed

| | Experi- | | Experi- | | Experi- | | Experi- | |
|---|---|---|---|---|---|---|---|---|
| | ment 1 | | ment 2 | | ment 3 | | ment 4 | |
| Temperature | 20° | C. | 50° | C. | 10° | C. | 20° | C. |
| Stirring Speed | 214 | rpm | 214 | rpm | 214 | rpm | 321 | rpm |
| OD600 | 0.064 | | 0.055 | | 0.062 | | 0.056 | |
| Depth filter | >400 | L/m² | >400 | L/m² | >400 | L/m² | >400 | L/m² |
| capacity | | | | | | | | |
| 0.45 micron | 177 | L/m² | 1367 | L/m² | 146 | L/m² | 171 | L/m² |
| Vmax | | | | | | | | |

A potential concern for the elevated temperature flocculation is the impact of elevated temperature on the 11A molecular structure and molecular weight. Serotype 11A has three O-acetyl groups, and one glycerol group connected to the polysaccharide repeat unit through phosphate. All of these groups could potentially cleave off during flocculation at low pH and elevated temperature. The other impact of the elevated temperature on the molecule is on the molecular weight. During flocculation at this condition (pH 3.5), the longer chain of polysaccharides may degrade to shorter chains, which could result in lower molecular weight. Our experiment shows that the product is identical to the original 11A purified after room temperature flocculation (see Table 24).

3. Centrifugation

Centrifugation has been conducted to clear centrate so that it can be filtered with reasonable capacity. The centrifugal speed was set at 12,000-xg.

4. Depth Filtration

Although centrifugation is the primary solid/liquid separation unit operation, it does not remove all of the particles from the feed stream, a depth filtration unit operation was incorporated between the centrifugation and the first ultrafiltation unit operation.

To determine the effect of broth hold time on depth filter capacity, centrate from a 20° C. flocculation using 2% alum and pH 3.5 was held for up to three days at 2-8° C. The centrate was then filtered using a depth filter and the filter capacity was determined. There were no significant differences in the depth filter capacity over the two day hold (Table 21).

TABLE 21
Broth Hold Time Impact on Filter Capacity

| Time | Day 0 | Day 1 | Day 2 |
|---|---|---|---|
| Filter Capacity (L/m²) | 193 | 185 | 243 |

5. Optional 0.45 Micron Filtration

Although optional, a 0.45 micron filter was used in some samples post depth filtration.

6. Ultrafiltration/Diafiltration-(UFDF-1)

Purification begins with the depth filtrate (from step 4 or 5 above).

This operation replaces the spent fermentation media with a buffer while reducing the levels of low molecular weight host cell impurities and residual floculant (aluminum).

In the development work for other serotypes (see above), it has been found that 10 mM sodium phosphate, 25 mM sodium citrate, pH 7.0 is a good buffer to use in the UF/DF process. Phosphate buffer is used to adjust the pH to neutral. Citrate buffer is used as a chelating agent to remove aluminum.

7. Carbon Filtration

This unit operation reduces the level of host cell impurities such as proteins and nucleic acids as well as colored impurities (see WO2008118752).

The UFDF1 retentate was filtered through a R32SP disc carbon filter. Results show that carbon has excellent capacity for the protein related impurity removal.

8. Ultrafiltration/Diafiltration-(UFDF-2)

This unit operation concentrates the product to the desired concentration and replaces the 25 mM sodium citrate, 10 mM sodium phosphate, pH 7.0 with the correct buffer for conjugation. This step is performed using a 30-kDa molecular weight cutoff filter.

The presence of residual citrate may interfere with the conjugation chemistry. To be highly reduced the level of citrate, diafiltration experiments were performed using different buffers (see examples 1 and 2).

9. Homogenization

Purified 11A polysaccharides can be homogenized, for example mechanically sized (see e.g. WO2015110942).

10. Sterile Filtration

The final unit operation prior to filling into storage bottles is a sterile filtration (0.2 micron filtration).

11. Consistency

To demonstrate that the recovery and purification process described above could produce reproducible results, three consistency batches were manufactured. The fermentation batch was flocculated and centrifuged using the process described above.

The step and overall yields for the three consistency batches are shown in Table 17.

TABLE 22
Step and Overall Yields for 11A Consistency Batches

| Unit Operation | 11A-001 | 11A-002 | 11A-003 |
|---|---|---|---|
| Fermentation | NA | NA | NA |
| Broth | | | |
| Centrate | 100 | 100 | 100 |
| Depth Filtrate | 89 | 89 | 92 |
| UFDF1 Retentate | 99 | 98 | 98 |
| Carbon Filtrate | 91 | 89 | 94 |
| UFDF2 Retentate | 95 | 98 | 56¹ |
| Homogenization | 99.9 | 96.7 | 91.6 |
| Overall Yield | 78 | 77 | 48 |

¹Product loss due to open valve

The analytical results for the three consistency batches are shown in Table 23.

TABLE 23
Analytical Results for 11A Consistency Batches

| Assay | 11A-001 | 11A-002 | 11A-003 |
|---|---|---|---|
| O-Acetytlation | 2.8/3.05 | 2.8/3.23 | 3.0/3.25 |
| Native/Sized | | | |
| Glycerol | 0.91/0.9 | 0.90/1.05 | 1.0/1.02 |
| Native/Sized | | | |
| Residual Citrate | <LOQ | <LOQ | <LOQ |
| Residual NLS | <LOQ | <LOQ | <LOQ |
| Residual Nucleic | <0.02% | <0.03% | <0.03% |
| Acids (w:w) | | | |
| Residual Protein | 0.2% | 0.2% | 0.3% |
| (w:w) | | | |
| Residual C poly | 1.5%/1.6% | 1.5%/1.6% | 2.1%/2.2% |
| Native/Sized | | | |
| Residual Al | <1 ppm | <1 ppm | <1 ppm |

Flocculation at different temperatures was conducted. One of the flocculation was performed at 50° C., and a portion of this material was purified using the above process. The analytical results from this purification along with the average results from the consistency batches are shown in Table 24. These results clearly show that increasing the flocculation temperature did not have any impact on product quality.

TABLE 24
Comparison of 50° C. Demo and Consistency Batches

| | 11A Consistency | | |
|---|---|---|---|
| Assay | Average (Room Temp) | 50° C. Batch | |
| O-Acetylation | 2.9 | 3.09 | |
| Residual Citrate | <LOQ | 9.6 | μg/ml |
| Residual NLS | <LOQ | N/A | |
| Residual Nucleic | 0.03% | 0.03% | |
| Acids | | | |
| Residual Protein | 0.2% | 0.0% | |
| Residual C poly | 1.7 wt % | 1.0 | wt % |

| | | |
|---|---|---|
| Glycerol | 0.94 | 0.96 |
| Overall Yield | 77% | 69% |

### Example 7. Purification of Pneumococcal Polysaccharide Serotype 12F

The process flow diagram for the purification of pneumococcal polysaccharide 12F is shown in FIG. 1. The process begins with NLS inactivated fermentation broth and includes recover unit operations (flocculation, centrifugation and depth filtration) followed by purification unit operations (ultrafiltration, and carbon filtration).

The homogenization step is optional.

1. Starting Material

The process begins with NLS inactivated fermentation broth of S. pneumonia serotype 12F. Cultures were grown in Hy-Soy medium. At the end of growth (as indicated by no further increase in optical density), the cultures were treated with NLS (see EP2129693).

2. Flocculation

The main purpose of this step is to precipitates cell debris, host cell proteins and nucleic acids. It also aids in the downstream clarification unit operations. The flocculation has been performed using fermentation broth that has been lysed by the addition of NLS.

Previous work on the purification process for other serotypes has shown that the clarification unit operation was impacted by the solution pH and the alum concentration, but not from the flocculation stirring time. In order to understand the impact of these factors on the flocculation of serotype 12F, a DOE study was conducted.

The ranges for each parameter are listed in Table 25.

TABLE 25
Parameter Range for Serotype 12F Flocculation DOE Study

| Range | Low | High |
|---|---|---|
| pH | 2 | 5 |
| Alum Concentration | 0% | 4% |
| Time | 30 min | 90 min |

A series of 16 experiments varying these 3 parameters were conducted (at room temperature).

The relationship between each variable (alum concentration, pH and flocculation time) and the responses (PS recovery, OD600 and protein removal) during the flocculation process has been analyzed by prediction profilers. The least important variable is flocculation time, which has almost no impact on either protein removal or polysaccharide recovery and a very minor impact on clarity, when the alum concentration and pH are set at their middle points.

Alum concentration mainly impacts the clarity. The optimal alum concentration is around 2.7%, which resulted in the best clarity. Although the OD600 difference is small when alum concentration is between 1.5% to 3.5%. Alum concentration has some impact on polysaccharide recovery. When it increases to about 4%, the polysaccharide recovery is slightly lower. Flocculation pH on the other hand mainly impacts protein removal, with higher the protein removal at lower pH. At pH 3.5 and below, the maximum impurity removal is achieved.

Work on other serotypes showed that increasing the temperature during flocculation resulted in higher depth filter capacity. When the flocculation temperature was increased to 50° C., the clarity of the centrate increased dramatically (OD600 decreased from 0.338 (at 20° C.) to 0.073 (at 50° C.)) and the filter capacity increased more than 8 times. When higher temperature was used for flocculation, the particle sizes shifted dramatically to the large particle size range, which then made the centrifugation easier.

Other experiments were also performed to understand the impact of flocculation temperature on the flocculation process. Serotype 12F fermentation broth was flocculated using 2% alum, pH 3.5 at two different temperatures for one hour. After flocculation, the broth was centrifuged and the clarity of the centrate was measured (OD600). The centrate was then filtered through depth filters to determine the filter capacity. The depth filter filtrate was further filtered through a 0.45 um dead-end filter using a Vmax model. Results from these experiments are shown in Table 26.

TABLE 26
Serotype 12F Flocculation with Different Temperatures

| | Experiment 1 | | Experiment 2 | | |
|---|---|---|---|---|---|
| Temperature | 20° C. | | 50° C. | | |
| Stirring Speed | 214 rpm | | 214 rpm | | |
| OD600 | 0.076 | | 0.030 | | |
| Filter | 1 | 2 | 1 | 2 | 3 |
| OD after | 0.022 | 0.014 | 0.019 | 0.014 | 0.020 |
| filtration | | | | | |
| Depth filter | >400 L/m² | >90 L/m² | >400 L/m² | >80 L/m² | >400 L/m² |
| capacity | | | | | |
| 0.45 micron | 503 L/m² | ≥1866 L/m² | 1490 L/m² | ≥1934 L/m² | ≥1985 L/m² |
| Vmax | | | | | |
| Vmax initial | 8503 LMH | 10129 LMH | 8555 LMH | 11085 LMH | 8495 LMH |
| flux | | | | | |

It can be seen from Table 26 that the OD600 is lower when 50° C. was used as the flocculation temperature. There was no difference in depth filter capacity for all the conditions as the capacity of the filter was not reached for all of them. However, 0.45 um Vmax data still show that higher temperature flocculation is much better.

3. Centrifugation

Centrifugation has been conducted to clear centrate so that it can be filtered with reasonable capacity. The centrifugal speed was set at 12,000-xg.

4. Depth Filtration

Although centrifugation is the primary solid/liquid separation unit operation, it does not remove all of the particles from the feed stream, a depth filtration unit operation was incorporated between the centrifugation and the first ultrafiltation unit operation.

5. Optional 0.45 Micron Filtration

Although optional, a 0.45 micron filter was used in some samples post depth filtration.

6. Ultrafiltration/Diafiltration-(UFDF-1)

Purification begins with the depth filtrate (from step 4 or 5 above).

This operation replaces the spent fermentation media with a buffer while reducing the levels of low molecular weight host cell impurities and residual floculant (aluminum).

In the development work for other serotypes (see above), it has been found that 10 mM sodium phosphate, 25 mM sodium citrate, pH 7.0 is a good buffer to use in the UF/DF process. Phosphate buffer is used to adjust the pH to neutral. Citrate buffer is used as a chelating agent to remove aluminum.

7. Carbon Filtration

This unit operation reduces the level of host cell impurities such as proteins and nucleic acids as well as colored impurities (see WO2008118752).

The UFDF1 retentate was filtered through a R32SP disc carbon filter. Results show that carbon has excellent capacity for the protein related impurity removal and the product recovery from carbon filtration is very good.

A 0.2 μm filtration post-carbon filtration has been performed for some samples (optional).

8. Ultrafiltration/Diafiltration-(UFDF-2)

This unit operation concentrates the product to the desired concentration and replaces the 25 mM sodium citrate, 10 mM sodium phosphate, pH 7.0 with the correct buffer for conjugation. This step is performed using a 30-kDa molecular weight cutoff filter.

The presence of residual citrate may interfere with the conjugation chemistry. To be highly reduced the level of citrate, diafiltration experiments were performed using different buffers (see examples 1 and 2).

9. Sterile Filtration

The final unit operation prior to filling into storage bottles is a sterile filtration (0.2 micron filtration).

10. Consistency

To demonstrate that the recovery and purification process described above could produce reproducible results, three consistency batches were manufactured. The fermentation batch was flocculated and centrifuged using the process described above.

The step and overall yields for the three consistency batches are shown in Table 27.

TABLE 27
Step and Overall Yields for 12F Consistency Batches

| Unit Operation | 12F-001 | 12F-002 | 12F-003 |
|---|---|---|---|
| Fermentation | NA | NA | NA |
| Broth | | | |
| Centrate | 100 | 100 | 100 |
| Depth Filtrate | 89 | 88 | 91 |
| UFDF1 Retentate | 97 | 97 | 96 |
| Carbon Filtrate | 83 | 86 | 82 |
| UFDF2 Retentate | 93 | 94 | 94 |
| Final filtration | 94 | 99 | 96 |
| Overall Yield | 63 | 67 | 65 |

The analytical results for the three consistency batches are shown in Table 28.

TABLE 28
Analytical Results for 12F Consistency Batches

| | | | |
|---|---|---|---|
| Assay | 11A-001 | 11A-002 | 11A-003 |

| | | | |
|---|---|---|---|
| Residual Citrate | <LOQ | <LOQ | <LOQ |
| Residual NLS | <LOQ | <LOQ | <LOQ |
| Residual Nucleic | <0.03% | <0.03% | <0.03% |
| Acids (w:w) | | | |
| Residual Protein | 0.5% | 0.5% | 0.5% |
| (w:w) | | | |

| | | | | | | |
|---|---|---|---|---|---|---|
| Residual C poly | 0.6 | wt % | 0.5 | wt % | 0.6 | wt % |
| Residual Al | 5 | ppm | 6 | ppm | 6 | ppm |

### Example 8. Purification of S. aureus Cp5 and Cp8 Polysaccharides

This example describes a purification process for the isolation of capsular polysaccharides type 5 (Cp5) and type 8 (Cp8) from Staphylococcus aureus.

1. Starting Material

The starting material for the purification process was the whole cell (unlysed) S. aureus fermentation harvest.

2. Acid Hydrolysis

Following the harvest of S. aureus fermentation, the whole-cell broth was adjusted to acidic pH by addition of strong acid (e.g. sulfuric acid), heated, and then incubated for a period of time (see WO2011041003). After hydrolysis, the broth was cooled and then neutralized by the addition of sodium hydroxide solution.

3. Flocculation

Flocculation was performed by addition of 10% (w/v) aqueous alum (sodium aluminum phosphate) solution to the cool (20-30° C.) neutralized broth (of step 2 above), with stirring, to generate a final 2% (w/v) alum solution in broth. The broth was neutralized (pH 6.9-7.1) by addition of sodium hydroxide solution (1-10 N). After neutralization, the flocculated broth was incubated at room temperature for at least 10 minutes prior to clarification by microfiltration.

4. Broth Clarification (Microfiltration or Centrifugation)

The flocculated broth was clarified by tangential flow microfiltration, using a 0.2 μm pore diameter hollow fiber membrane. The desired product of this clarification was the permeate from both the concentration and the diafiltration stages; the retentate is ultimately discarded. The flocculated broth was concentrated approximately 4-fold under constant flux conditions at a shear rate of 4000-8000 s−1. After concentration, constant-volume diafiltration (5 diavolumes) was performed against deionized water. Diafiltration is also performed under constant flux conditions.

After diafiltration, the combined permeate from both concentration and diafiltration stages served as the feed for the next operation.

5. Ultrafiltration/Diafiltration-(UFDF-1)

The microfiltration permeate was concentrated and diafiltered using a hollow fiber tangential flow ultrafiltration membrane. The retentate was collected as product; permeate was discarded as waste. The feed (microfiltration permeate) was concentrated approximately 8-15 fold. After concentration, the retentate was diafiltered (constant-volume) against at least 10 diavolumes of 125 mM sodium phosphate, pH 7.5) buffer.

After diafiltration, the retentate was recovered by draining from the filter apparatus.

Alternatively, centrifugation can also be used as a clarification method to separate the precipitated cell debris in the flocculated broth from liquid. The supernatant can then be processed via subsequent carbon filtration step.

6. Carbon Filtration

The ultrafiltration/diafiltration retentate next were filtered using carbon filtration. For both Cp5 purification and Cp8 purification, Cuno R32SP-grade carbon filter were used. The retentate was typically fed through the carbon filter(s) in single-pass operation. The carbon filtrate was collected as product. After product filtration, the carbon was rinsed with 125 mM sodium phosphate (pH 7.5) buffer. This rinse was combined with the product filtrate, and proceeds to the periodate oxidation.

7. Periodate Oxidation

The combined carbon filtrate and filter rinse next undergo an oxidation reaction with periodate. At room temperature, 1.0 M solution of periodic acid is added to carbon filtrate/rinse from the previous purification step (generating 50 mM final concentration of periodate). This reaction mixture was incubated at room temperature for 30 minutes. Then, a molar excess of propylene glycol was added to the reaction mixture to quench the reaction. Following the quench, the reaction products were neutralized (pH 6.9-7.1) by addition of sodium hydroxide. The reaction product solution then proceeds to the final ultrafiltration/diafiltration operation.

8. Ultrafiltration/Diafiltration-(UFDF-2)

The periodate oxidation product mixture was concentrated and diafiltered by means of a hollow fiber tangential flow ultrafiltration membrane. The material was concentrated 2-4-fold (to about 4-8 g/L Cp5/Cp8) under constant TMP conditions and a constant shear rate. Next, the retentate is diafiltered (constant-volume) against at least 10 diavolumes of DI water.

After diafiltration, the retentate was recovered, and the filter was then rinsed using minimal volume of DI water. The rinse was drained and collected with the retentate; the combined material was then sterile filtered.

9. Sterile Filtration

The combined retentate and rinse was filtered through an appropriately sized dead-end sterilizing-grade filter (0.2 μm pore) into a sterile container. This filtrate was then stored at 4° C.

All publications and patent applications mentioned in the specification are indicative of the level of those skilled in the art to which this invention pertains. All publications and patent applications are hereby incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

1. A method for purifying a bacterial polysaccharide from a solution comprising said polysaccharide together with contaminants, wherein said method comprises a flocculation step comprising the addition of a flocculating agent.

2. The method of claim 1 wherein the flocculating agent comprises a multivalent cation selected from aluminium, iron, calcium and magnesium.

3. The method of claim 1 wherein the flocculating agent comprises an agent selected from alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate.

4. The method of claim 1 wherein the flocculating agent is selected from alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.

5. The method of claim 1 wherein the concentration of flocculating agent is between about 0.1 and about 20% (w/v).

6. The method of claim 1 wherein the solution is hold for some time to allow settling of the flocs prior to downstream processing.

7. The method of claim 6 wherein said flocculation step is performed at an acidic pH.

8. The method of claim 6 wherein the settling step, if present, is performed at a temperature between about 4° C. and about 30° C.

9. The method of claim 6 wherein the settling step, if present, is performed at a temperature of between about 30° C. to about 95° C.

10. The method of claim 1 wherein, following flocculation the suspension is clarified by decantation, sedimentation, filtration or centrifugation.

11. The method of claim 10 wherein, the polysaccharide containing solution is filtrated.

12. The method of claim 11 wherein, said filtration is depth filtration.

13. The method of claim 11 wherein the filtrate is subjected to microfiltration.

14. The method of claim 11 wherein the filtrate is further treated by Ultrafiltration and Diafiltration.

15. The method of claim 14 wherein said ultrafiltration step is performed at temperature between about 20° C. to about 90° C.

16. The method of claim 14 wherein the diafiltration comprises a replacement solution comprising a chelating agent.

17. The method of claim 14 wherein said diafiltration step is performed at temperature of between about 20° C. to about 90° C.

18. The method of claim 10 wherein the solution containing the polysaccharide is treated by an activated carbon filtration step.

19. The method of claim 18, wherein the filtrate is subjected to microfiltration.

20. The method of claim 18, wherein the filtrate is further clarified by ultrafiltration and diafiltration.

21. The method of claim 20 wherein the diafiltration comprises a replacement solution comprising a chelating agent.

22. The method of claim 20 wherein said diafiltration step is performed at temperature of between about 20° C. to about 90° C.

23. The method of claim 1 wherein said purified solution of polysaccharide is homogenized by sizing.

24. The method of claim 1 wherein said purified solution of polysaccharide is sterilely filtered.

25. The method of claim 1 wherein said bacterial polysaccharide is a capsular polysaccharide.

26. A glycoconjugate of a purified bacterial polysaccharide obtained by the method of claim 1.

27. An immunogenic composition comprising a glycoconjugate of claim 26.
